(19) United States
(12) Patent Application Publication
(10) Pub. No.: US 2023/0372491 A1
(43) Pub. Date: Nov. 23, 2023

(54) ENDOSOMAL ESCAPE DOMAINS FOR DELIVERY OF MACROMOLECULES INTO CELLS

(71) Applicant: The Regents of the University of California, Oakland, CA (US)

(72) Inventors: Steven F. Dowdy, La Jolla, CA (US); Satish G. Jadhav, San Diego, CA (US)

(21) Appl. No.: 18/029,470

(22) PCT Filed: Oct. 12, 2021
(86) PCT No.: PCT/US2021/054604

§ 371 Date: Mar. 30, 2023

Related U.S. Application Data
(60) Provisional application No. 63/090,551, filed on Oct. 12, 2020.
(60) Provisional application No. 63/208,416, filed on Jun. 8, 2021.

(51) Int. Cl. A61K 47/54; C12N 15/113; C12N 15/88

(52) U.S. Cl. CPC A61K 47/549; C12N 15/113; C12N 15/88; A61K 47/545; C12N 2310/14; C12N 2310/3515; C12N 2310/3513; C12N 2310/314

(57) ABSTRACT

The disclosure provides for compounds and compositions comprising universal endosomal escape domains, and applications thereof, including for delivering macromolecules into cells.

### CROSS REFERENCE TO RELATED APPLICATIONS

The application claims priority under 35 U.S.C. § 119 to U.S. Provisional Application Ser. No. 63/090,551, filed Oct. 12, 2020; and U.S. Provisional Application Ser. No. 63/208,416, filed Jun. 8, 2021, the disclosures of which are incorporated herein by reference.

### STATEMENT REGARDING FEDERALLY SPONSORED RESEARCH

This invention was made with government support under Grant Nos. R21-CA25251, CA234740, and NS11663 awarded by the National Institutes of Health (NIH). The government has certain rights in the invention.

### TECHNICAL FIELD

The disclosure provides for compounds and compositions comprising universal endosomal escape domains, and applications thereof, including for delivering macromolecules (siRNAs, ASOs, Oligonucleotides, CRISPR DNA/RNA Editing Enzymes, mRNA, DNA vectors, proteins, peptides, antibodies, Lipid Nanoparticles, etc.) into cells.

### BACKGROUND

Eukaryotic cells contain several thousand proteins, which have been, during the course of evolution, selected to play specific roles in the maintenance of virtually all cellular functions. Not surprisingly, the viability of every cell, as well as the organism on the whole, is intimately dependent on the correct expression of these proteins. Factors which affect a particular protein's function, either by mutations or deletions in the amino acid sequence, or through changes in expression to cause over-expression or suppression of protein levels, invariably lead to alterations in normal cellular function. Such alterations often directly underlie a wide variety of genetic and acquired disorders. Consequently, the ability to target and selectively inhibit, alter or increase expression of genes or kill cells comprising mutations that result in cell proliferative disorders would help to control such diseases and disorders.

In practice, however, the direct intracellular delivery of these agents has been difficult. This is due primarily to the bioavailability barrier of the cell's lipid bilayer plasma membrane, which effectively prevents the uptake of the majority of peptides, proteins, RNAs, DNAs, CRISPR and other agents by limiting their passive entry. Even if the agent is able to be taken up by a cell through endosomal uptake mechanisms, due to the endosomal lipid bilayer membrane, the release of the agent intracellularly is still a rate limiting step (e.g., see FIG. 1).

### SUMMARY

The disclosure provides methods and compositions useful for delivery of molecules into cells. The disclosure provides compositions that comprise endosomal escape domains which exhibit improved escape from the endosome of transported cargo. In particular, the disclosure provides for universal Endosomal Escape Domain (uEED) compositions which comprise a hydrophilic mask domain linked to a cargo molecule and further linked to a biodegradable linker that separates the hydrophilic mask domain from a hydrophobic or cationic domain. Described herein is the syntheses of multiple classes of uEED phosphoramidite building block monomers, syntheses of multiple uEED multimers, and the conjugation of uEED multimers to oligonucleotides. The disclosure further characterizes the metabolic stability of the uEED to serum enzymes and more importantly, the selective cleavage of uEEDs by endosomal/lysosomal restricted enzymes (e.g., β-glucuronidases and other glucosidases), which selectively cleaved away the hydrophilic mask domain from the hydrophobic and/or cationic domain, thereby selectively activating the uEED inside of the endosome.

In a particular embodiment the disclosure provides for a monomeric compound comprising: a coupler domain; a hydrophobic domain or a cationic charge domain; a hydrophilic mask domain; a biodegradable linker having a first end and a second end, wherein the endosomal cleavable linker is linked to the hydrophilic mask domain on the first end, and linked to the hydrophobic domain or cationic charge domain on the second end, or linked to a first linker on the second end; a first linker having a first end and a second end, wherein the first linker is linked to the coupler domain on the first end, and linked on the second end to the hydrophobic domain or cationic charge domain, or linked to a second linker; optionally, a second linker having a first end and a second end, wherein the second linker is linked to the hydrophobic domain or a cationic charge domain on the first end, and linked to the first linker on the second end; optionally, third and/or fourth linkers having a first end and a second end, wherein the first end is attached to the coupler domain, wherein the second end is attached to a functional group for solid-state synthesis. In another embodiment, the monomeric compound has the structure of Formula I, II, III or IV:

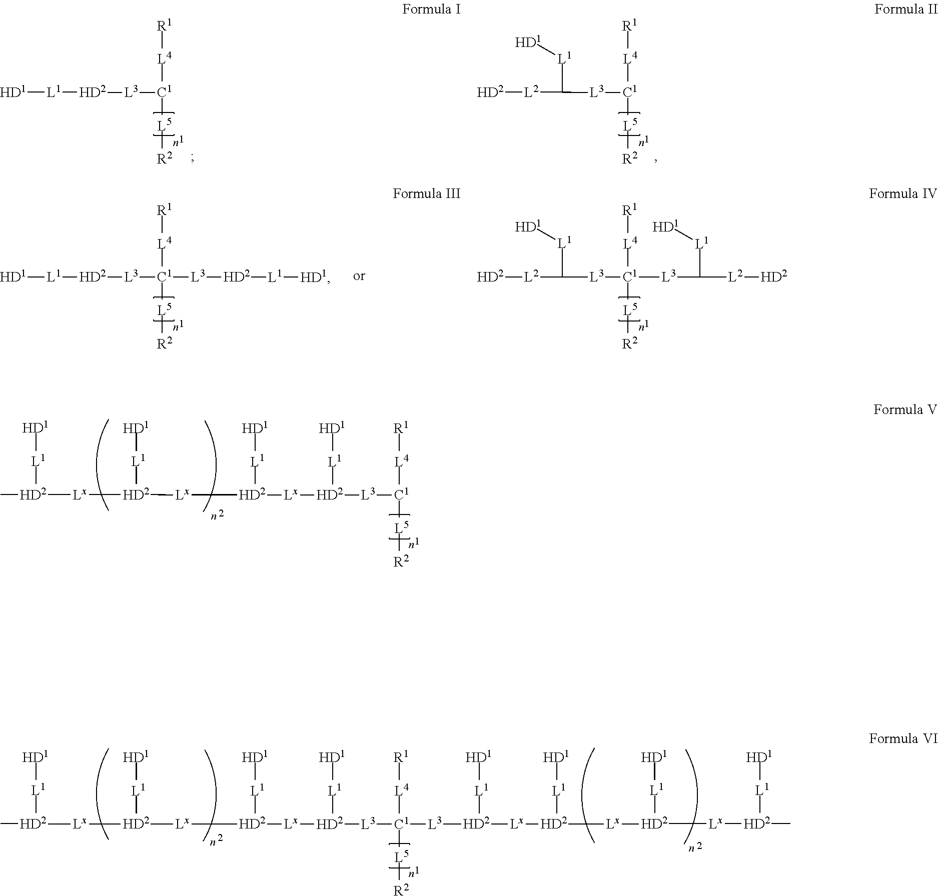

or a pharmaceutically acceptable salt, or solvate thereof, wherein, C¹ is the coupler domain; HD¹ is the hydrophilic mask domain; HD² is the hydrophobic domain or a cationic charge domain; L¹ is the biodegradable linker; L² is the second linker; L³ is the first linker; L⁴ is the third linker; L⁵ is the fourth linker (wherein the L4 and L5 linkers can have different numbers of carbons or other atoms); R¹ and R² are protecting of functional groups for solid-state synthesis; and n¹ is an integer selected from 0 or 1; n² is an integer selected from 0 to 10 or more (e.g., 1, 2, 3, 4, 5, 6, 7, 8, 9, 10, 11 etc.). In a further embodiment, the coupler domain comprises a phosphotriester group, or phosphoramidite group. In yet a further embodiment, the hydrophilic mask domain comprises a glycoside moiety or a protein transduction/cell penetrating peptide. In another embodiment, the hydrophobic domain or the cationic charge domain is any functional group (or a plurality of such functional groups) which contains a primary, secondary or tertiary amino group; a lipid or a monomeric unit derived therefrom; a tocopherol; a hydrophobic oligomer or a monomeric unit derived therefrom; a hydrophobic polymer or a monomeric unit derived therefrom. In yet another embodiment, the hydrophobic domain comprises a lipid selected from a C8, C10, C12, C14, C16, or C18 lipid or derivative thereof. In a further embodiment, the hydrophobic domain comprises a monomeric unit derived from a lipid selected from fatty acid, fatty alcohol and any other lipidic molecule with at least two carbon units. In yet a further embodiment, the hydrophobic domain comprises a hydrophobic polymer selected from polymethylacryl, polyethylene, polystyrene, polyisobutane, polyester, polypeptide, or a derivative thereof. In another embodiment, the hydrophobic domain comprises one or more monomeric units derived from a hydrophobic polymer selected from the group consisting of: polyester, polyether, polycarbonate, polyanhydride, polyamide, polyacrylate, polymethacrylate, polyacrylamide, polysulfone, polyalkane, polyalkene, polyalkyne, polyanhydride, polyorthoester, N-isopropylacrylamide, methyl acrylate, ethyl acrylate, propyl acrylate, butyl acrylate, methyl methacrylate, ethyl methacrylate, propyl methacrylate, butyl methacrylate, acrylic acid, methacrylic acid, quaternary ammonium-modified acrylate, quaternary ammonium modified-methacrylate, acrylamide, caprolactone, lactide, and valerolactone. In yet another embodiment, the hydrophobic domain or the cationic charge domain comprises a 1H-indole group. In another embodiment, the biodegradable linker comprises a thioether group, a carbamate group, an ester group, a carbonate group, a urea group, or an enzyme cleavable peptidic linkage. In yet another embodiment, the biodegradable linker is an endosomal cleavable linker. In a certain embodiment, the endosomal cleavable linker comprises a carbamate group or a hydrazone group. In a further embodiment, the first linker comprises a group selected from an optionally substituted (C₁-C₆)alkyl, an optionally substituted (C₂-C₆)alkenyl, an optionally substituted (C₂-C₆)alkynyl, or an optionally substituted (C₁-C₆) alkoxy group. In another embodiment, the first linker comprises a group selected from ethyl, propyl, PEG₂, PEG₃ and PEG₄. In a further embodiment, the second linker is selected from an optionally substituted (C₁-C₆)alkyl, an optionally substituted (C₂-C₆)alkenyl, an optionally substituted (C₂-C₆)alkynyl, or an optionally substituted (C₁-C₆) alkoxy group. In yet a further embodiment, the second linker comprises a group selected from ethyl, propyl, PEG₂, PEG₃ and PEG₄. In another embodiment, the third and fourth linkers are selected from an optionally substituted (C₁-C₆)alkyl, an optionally substituted (C₂-C₆)alkenyl, an optionally substituted (C₂-C₆)alkynyl, an optionally substituted (C₁-C₆) alkoxy group, a uridine group, and a pyrimidine group. In a certain embodiment, the third and fourth linkers are a (C₁-C₆) alkyl or a uridine group. In another embodiment, the functional or protecting groups for solid-state synthesis are an amidite and a 4,4′-dimethoxytrityl group. In yet another embodiment, the compound has a structure selected from:

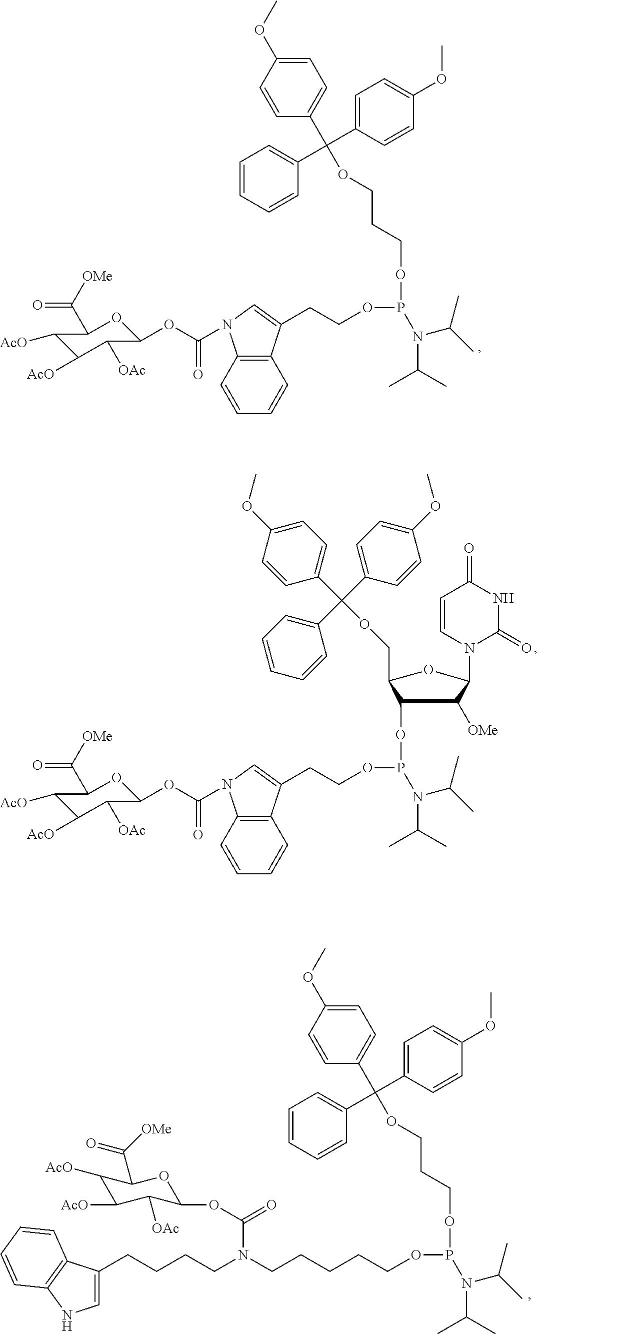

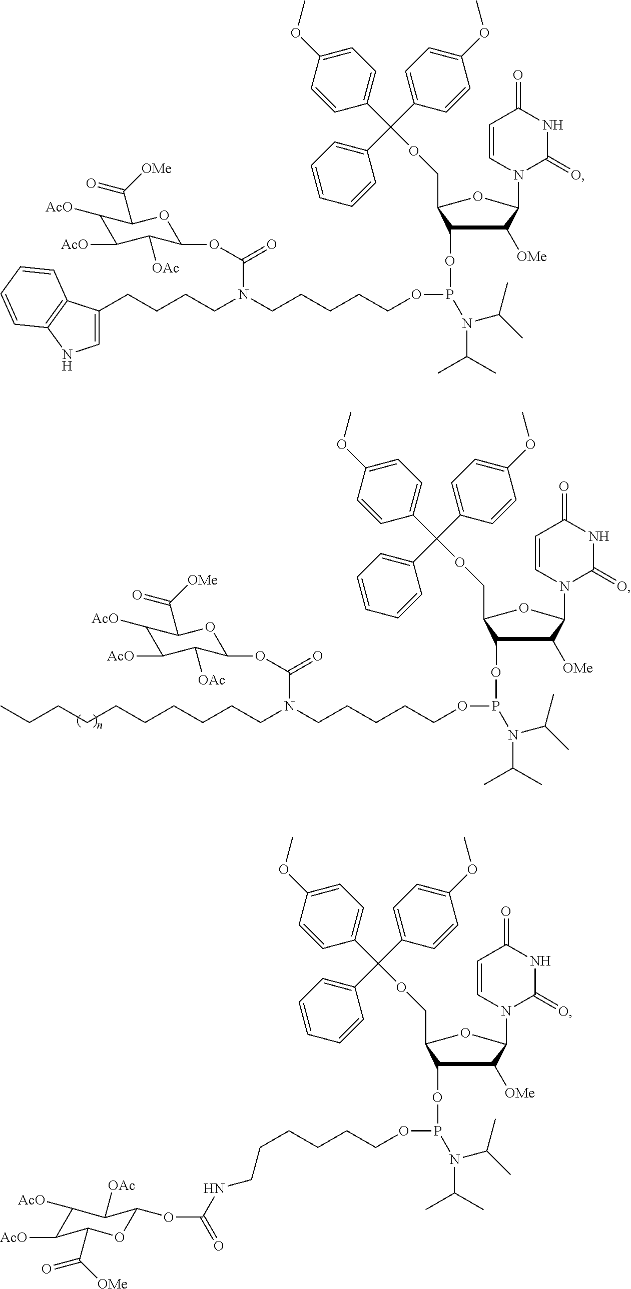

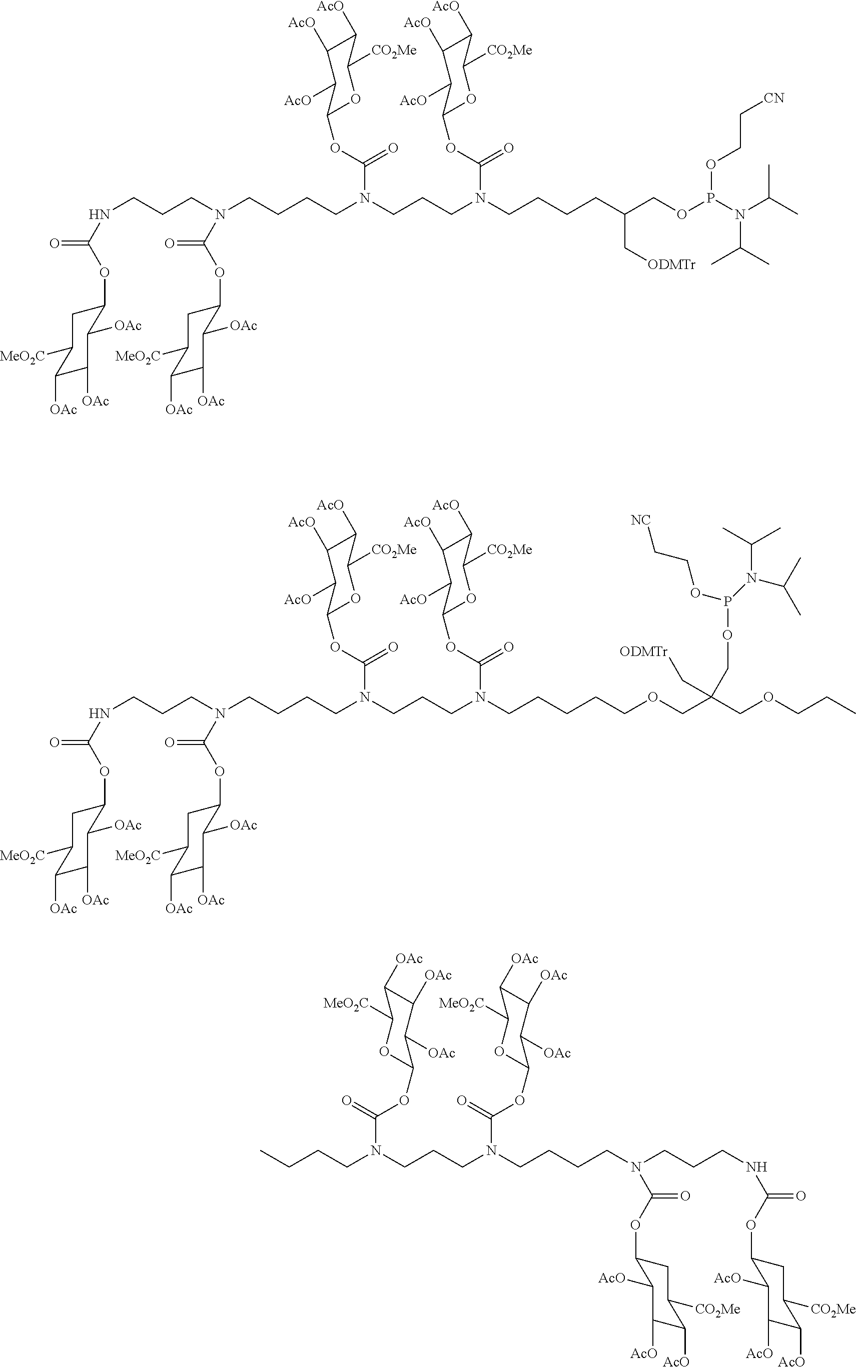

In a certain embodiment, the disclosure also provides for a multimeric compound comprising a plurality of monomeric compounds disclosed herein, wherein the plurality of monomeric compounds have been linked together using solid-state synthesis to form a multimeric compound. In a further embodiment, the multimeric compound is linked to a cargo molecule. In yet a further embodiment, the cargo molecule is selected from the group consisting of a small molecule therapeutic, a peptide, a protein, a single stranded oligonucleotide, a double stranded oligonucleotide, and a protein-oligonucleotide complex, e.g., CRISPR DNA/RNA editing, mRNA, DNA vectors and Lipid nanoparticles. In another embodiment, the cargo molecule is linked to the multimeric compound by a covalent bond, hydrogen bonds, or by electrostatic attraction.

In a particular embodiment, the disclosure further provides a multimeric compound having a structure of Formula VII:

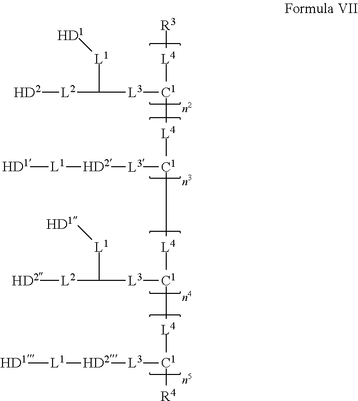

or a pharmaceutically acceptable salt, or solvate thereof, wherein, C¹ is a coupler domain; HD¹, HD^{1′}, HD^{1″}, and HD^{1′″} are each individually selected hydrophilic domains or cationic mask domains; HD², HD^{2′}, HD^{2″}, and HD^{2′″} are each individually selected hydrophobic domains or cationic charge domains; L^{1′} is a biodegradable linker; L² is a second linker; L³ is a first linker; L⁴ is a third linker; R³ is an H or a conjugation handle for a cargo molecule; R⁴ is an H or a conjugation handle for a cargo molecule; n² is an integer selected from 0 to 10; n³ is an integer selected from 0 to 10; n⁴ is an integer selected from 0 to 10; and n⁵ is an integer selected from 0 to 10; wherein, the summation of the integers specified for n¹ to n⁵ is from 4 to 30, and wherein at least one of R³ and R⁴ is a conjugation handle for a cargo molecule. In a further embodiment, the coupler domain comprises a phosphotriester group. In yet a further embodiment, the hydrophilic mask domains comprise a glycoside moiety or a protein transduction/cell penetrating peptide. In another embodiment, the hydrophobic domains or the cationic charge domains are selected from any functional group which contains a primary, secondary or tertiary amino group; a lipid or a monomeric unit derived therefrom; a tocopherol; a hydrophobic oligomer or a monomeric unit derived therefrom; a hydrophobic polymer or a monomeric unit derived therefrom. In yet another embodiment, one or more of the hydrophobic domains comprise a lipid selected from a C8, C10, C12, C14, C16, or C18 lipid or derivative thereof or an aromatic compound comprising a single, double, triple, or extended ring structure. In a certain embodiment, one or more of the hydrophobic domains comprise a monomeric unit derived from a lipid selected from fatty acid, fatty alcohol and any other lipidic molecule with at least two carbon units. In another embodiment, one or more of the hydrophobic domains comprise a hydrophobic polymer selected from polymethylacryl, polyethylene, polystyrene, polyisobutane, polyester, polypeptide, or a derivative thereof. In yet another embodiment, one or more of the hydrophobic domains comprise one or more monomeric units derived from a hydrophobic polymer selected from the group consisting of: polyester, polyether, polycarbonate, polyanhydride, polyamide, polyacrylate, polymethacrylate, polyacrylamide, polysulfone, polyalkane, polyalkene, polyalkyne, polyanhydride, polyorthoester, N-isopropylacrylamide, methyl acrylate, ethyl acrylate, propyl acrylate, butyl acrylate, methyl methacrylate, ethyl methacrylate, propyl methacrylate, butyl methacrylate, acrylic acid, methacrylic acid, quaternary ammonium-modified acrylate, quaternary ammonium modified-methacrylate, acrylamide, caprolactone, lactide, and valerolactone. In a certain embodiment, one or more of the hydrophobic domains or the cationic charge domains comprise a 1H-indole group. In another embodiment, the biodegradable linker comprises a thioether group, a carbamate group, an ester group, a carbonate group, a urea group, or an enzyme cleavable peptidic linkage. In yet another embodiment, the biodegradable linker is an endosomal cleavable linker. In a further embodiment, the endosomal cleavable linker comprises a carbamate group or a hydrazone group. In yet a further embodiment, the first linker comprises a group selected from an optionally substituted (C₁-C₆)alkyl, an optionally substituted (C₂-C₆)alkenyl, an optionally substituted (C₂-C₆)alkynyl, or an optionally substituted (C₁-C₆) alkoxy group. In another embodiment, the first linker comprises a group selected from ethyl, propyl, PEG₂, PEG₃ and PEG₄. In yet another embodiment, the second linker is selected from an optionally substituted (C₁-C₆)alkyl, an optionally substituted (C₂-C₆)alkenyl, an optionally substituted (C₂-C₆)alkynyl, or an optionally substituted (C₁-C₆) alkoxy group. In a further embodiment, the second linker comprises a group selected from ethyl, propyl, PEG₂, PEG₃ and PEG₄. In yet a further embodiment, the third linker is selected from an optionally substituted (C₁-C₆)alkyl, an optionally substituted (C₂-C₆)alkenyl, an optionally substituted (C₂-C₆)alkynyl, an optionally substituted (C₁-C₆) alkoxy group, a uridine group, and a pyrimidine group. In another embodiment, the third linker is a (C₁-C₆) alkyl or a uridine group. In yet another embodiment, the conjugation handle for a cargo molecule comprises an azide group. In a particular embodiment, the conjugation handle for a cargo molecule comprises a structure of:

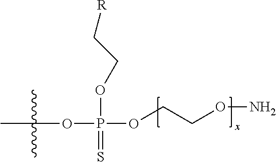

wherein, x is an integer selected from 1 to 15; and R is —OH, or —CN. In another embodiment, the conjugation handle comprises a terminal azide. In another embodiment, the multimeric compound is linked to a cargo molecule. In yet another embodiment, the cargo molecule is selected from the group consisting of a small molecule therapeutic, a peptide, a protein, a single stranded oligonucleotide, a double stranded oligonucleotide, and a protein-oligonucleotide complex. In a further embodiment, the cargo molecule is linked to the conjugation handle of the multimeric compound.

The details of one or more embodiments are set forth in the accompanying drawings and the description below. Other features, objects, and advantages will be apparent from the description and drawings, and from the claims.

### BRIEF DESCRIPTION OF THE FIGURES

FIG. 1 shows a general process of agent uptake and the difficulties and rate limiting step of endosomal escape.

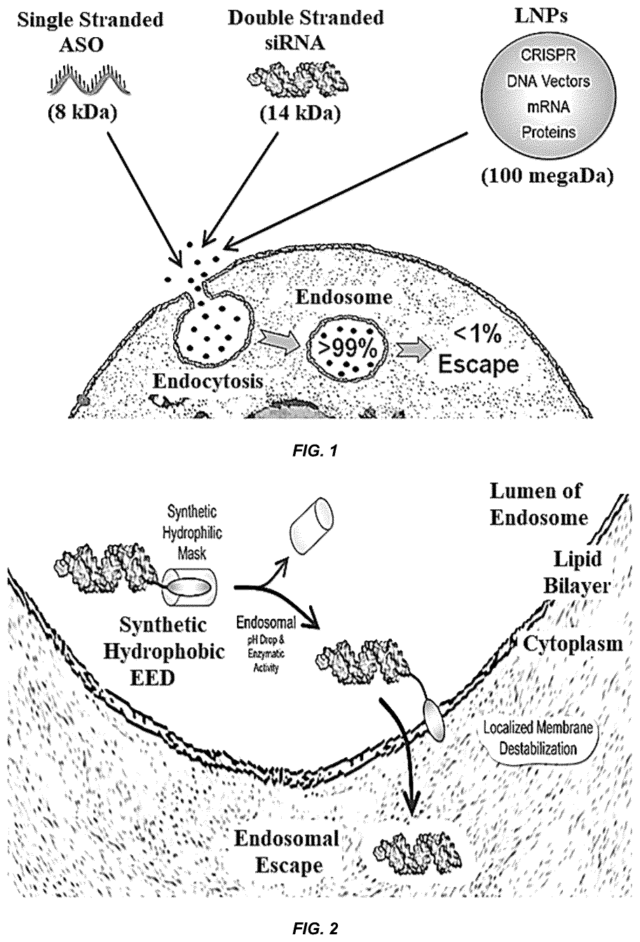

FIG. 2 shows a schematic example of how a composition of the disclosure generally works.

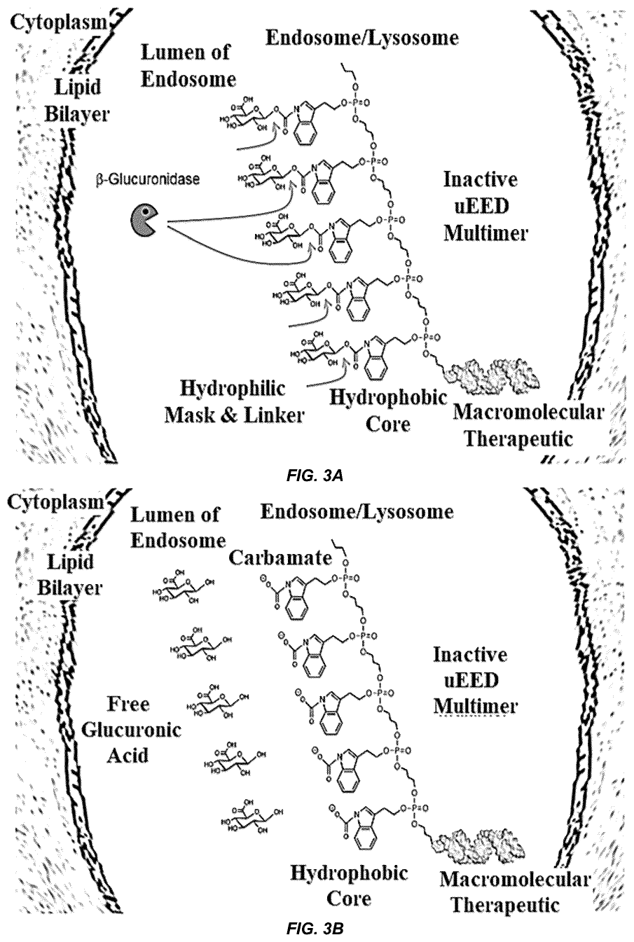

FIG. 3A-E depicts the activation of a universal endosomal escape domain (uEED) of the disclosure. (A) Depicts the cleavage of the hydrophilic mask at the linker between the hydrophilic mask and the hydrophobic core by an enzyme (e.g., β-glucuronidase. (B) Depicts the resulting product of (A) with a CO₂ remaining on the hydrophobic core. (C) shows the self-immolation of the CO₂ from the hydrophobic core. (D) depicts the activated uEED. (E) depicts the insertion of the hydrophobic core into the endosomal membrane which results in the destabilization and release of the cargo into the cytoplasm.

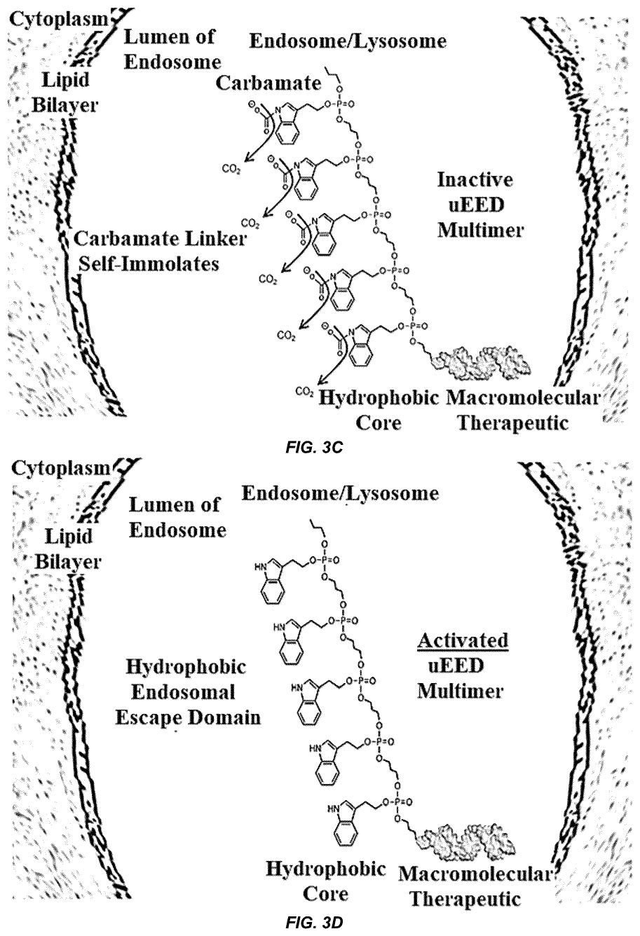

FIG. 4 provides an embodiment of a phosphoramidite uEED precursor that is made using solid-state synthesis that can be used to create the uEEDs of the disclosure. As shown, the phosphoramidite is connected to the hydrophobic core via a linker that is connected to the hydrophilic mask via a biodegradable linker.

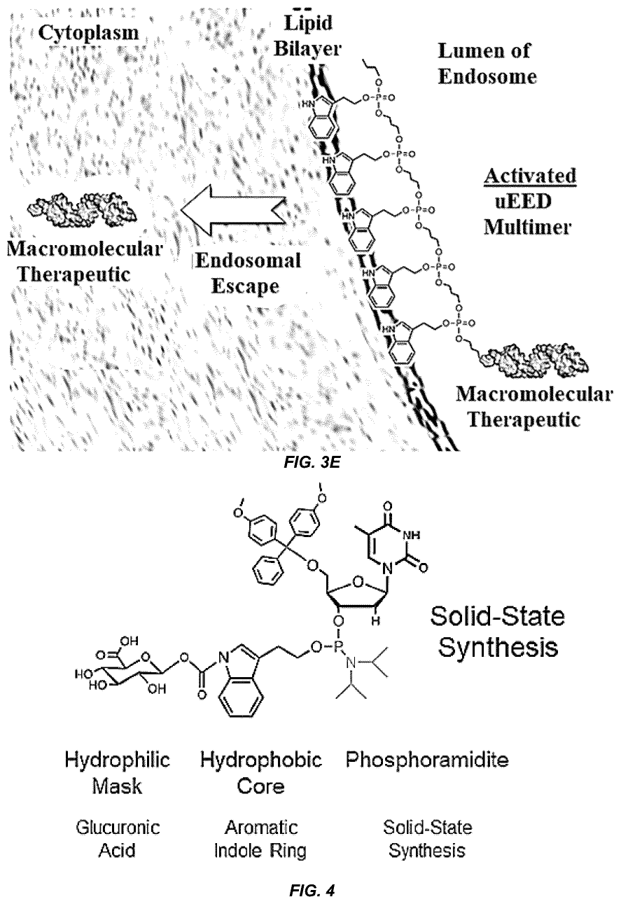

FIG. 5 provides an embodiment of a uEED multimer that is made by linking phosphoramidite uEED precursors together using solid-state synthesis techniques.

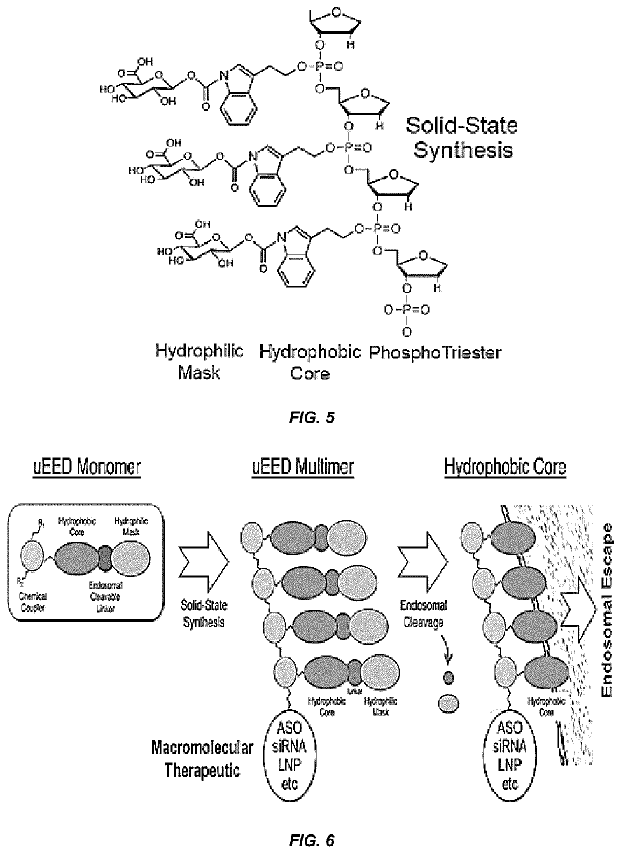

FIG. 6 shows schematics of one embodiment of the disclosure. The figure provides a monomeric structure of a uEED as well as uEED multimers linked to a cargo domain (e.g., ASO, siRNA, LNP, etc.). The figure also depicts the cleavage of the linker to release the hydrophilic mask from the hydrophobic core.

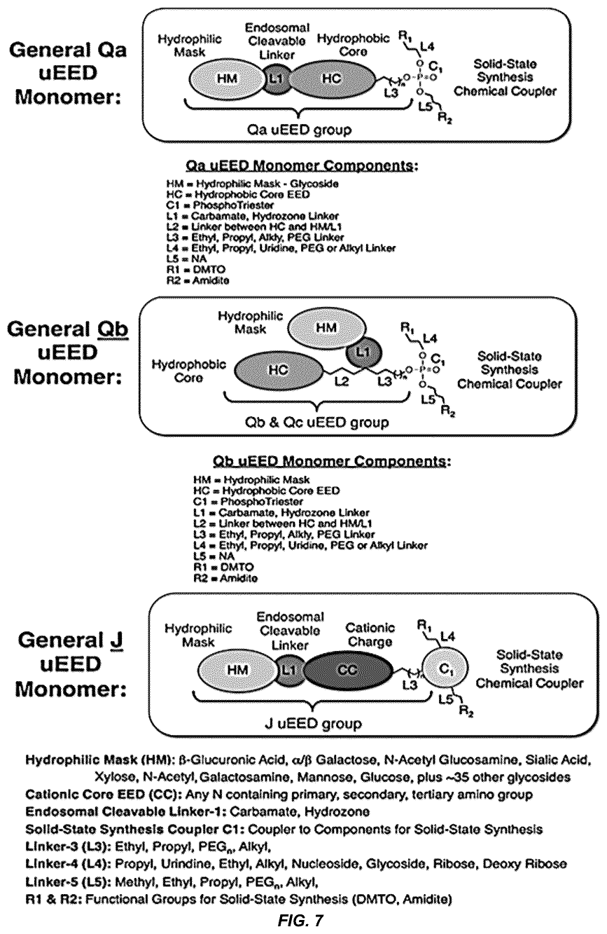

FIG. 7 shows various exemplary configurations and permutations of uEED monomers of the disclosure.

FIG. 8 diagrams how the uEED monomers (e.g., Qa, Qb, Qc, and J) are linked together to form uEED multimers, including various combinations of uEED monomers that have different hydrophobic cores.

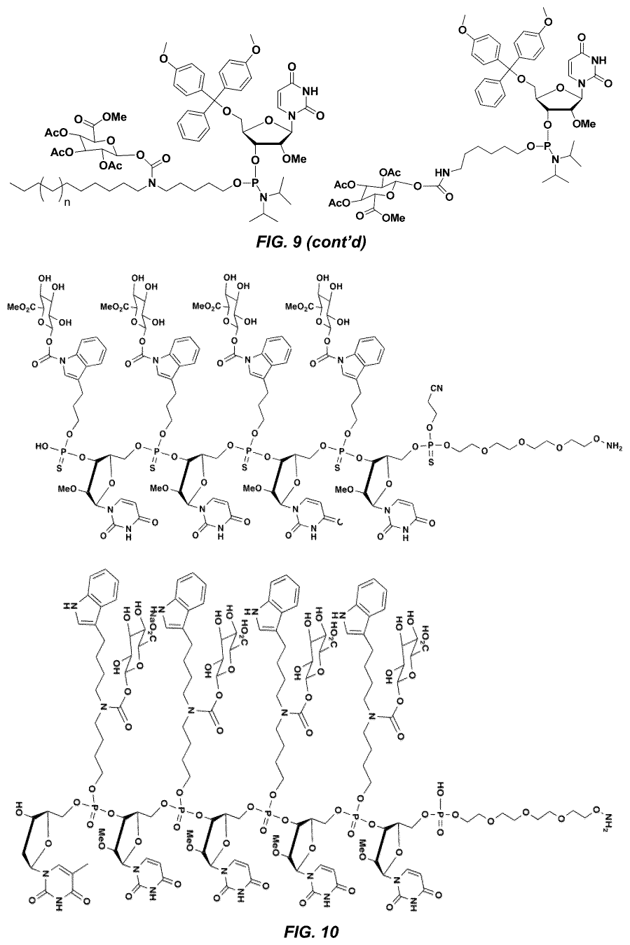

FIG. 9 provides embodiments of structures of phosphoramidite uEED precursors that can be used to make uEED multimers of the disclosure.

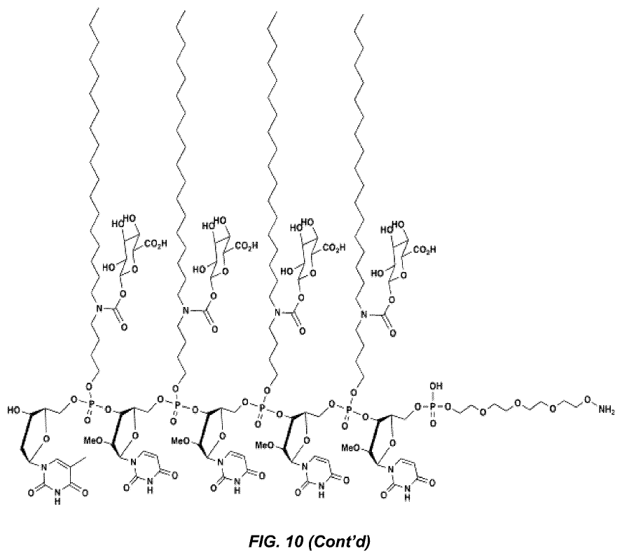

FIG. 10 provides embodiments of structures of uEED monomers that have been linked together to make exemplary uEEDs multimers.

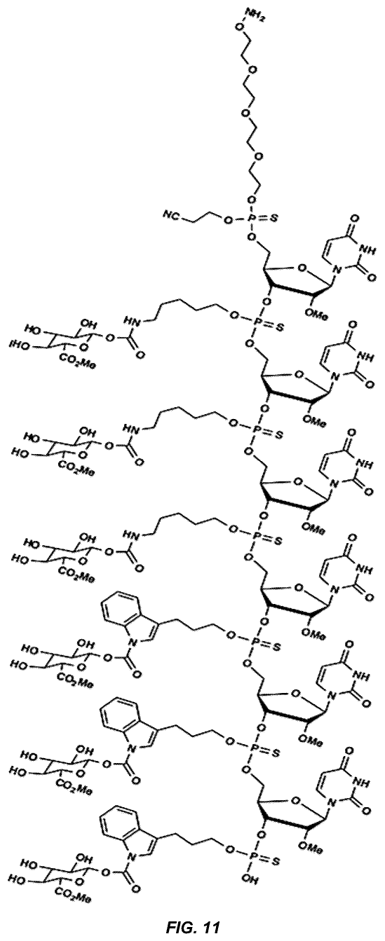

FIG. 11 provides embodiments of structures of uEED monomers comprising different hydrophobic cores that have been linked together to make exemplary uEEDs multimers.

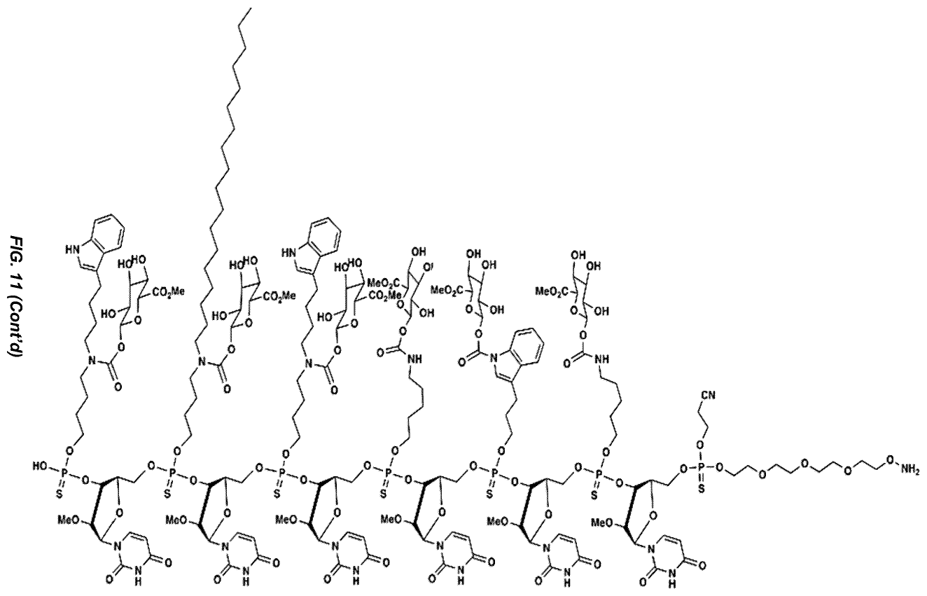

FIG. 12 provides a synthetic route to make a B-Gluc-P-Qa phosphoramidite uEED precursor.

FIG. 13 provides a synthetic route to make a B-Gluc-U-Qa phosphoramidite uEED precursor.

FIG. 14 provides a synthetic route to make a galactose-P/U-Qa phosphoramidite uEED precursors.

FIG. 15 provides for the synthesis of a B-Gluc-U-Qa uEED multimer.

FIG. 16 provides a synthetic route to make a B-Gluc-P-Qb phosphoramidite uEED precursor.

FIG. 17 provides a synthetic route to make a B-Gluc-U-Qb phosphoramidite uEED precursor.

FIG. 18 provides a synthetic route to make a galactose-P/U-Qb phosphoramidite uEED precursors.

FIG. 19 provides a synthetic route to make a B-Gluc-P-Qc uEED phosphoramidite precursor.

FIG. 20 provides a synthetic route to make a B-Gluc-U-Qc uEED phosphoramidite precursor.

FIG. 21 provides a synthetic route to make a B-Gluc-P-J uEED phosphoramidite precursor.

FIG. 22 provides a synthetic route to make a B-Gluc-U-J uEED phosphoramidite precursor.

FIG. 23 provides a synthetic route to make a B-Gluc-P/U-J uEED phosphoramidite precursors.

FIG. 24 provides a diagram indicating how the uEEDs of the disclosure can be used to facilitate intracellular delivery of all types of macromolecules (e.g., single stranded oligonucleotides, double stranded RNAi Triggers, proteins, peptides, and gene editing components, mRNA, DNA vectors, Lipid Nanoparticles etc.). All intracellular macromolecular therapeutics (including: siRNAs, ASOs, RNPs, Peptides, Proteins, mRNA, CRISPR, DNA vectors, Large Synthetic Molecules, etc.) are taken up into cells by endocytosis. However, >99% remains trapped inside the endosome. The uEED directly addresses this problem to drive endosomal escape of the macromolecular cargo into the cytoplasm and nucleus of the cell. uEEDs can be conjugated to all macromolecular therapeutic classes.

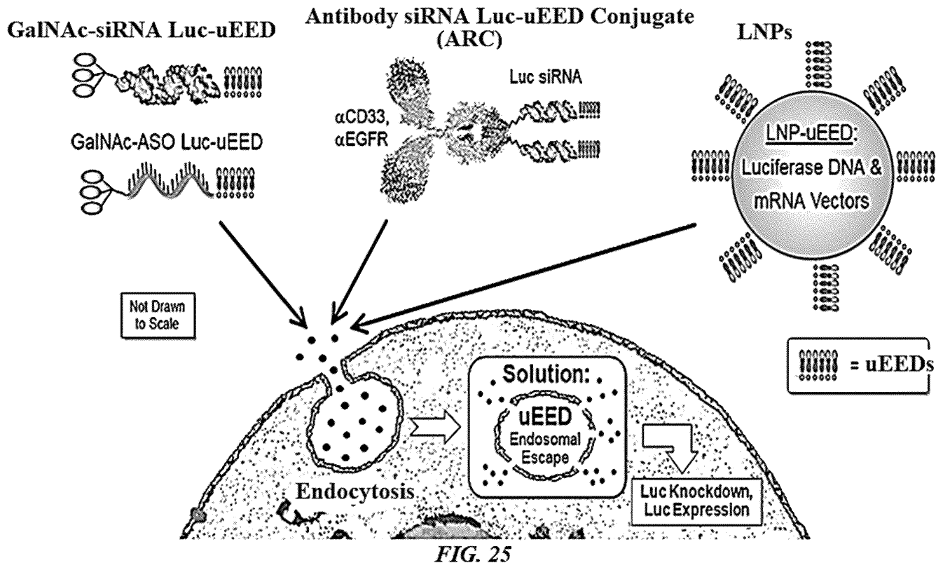

FIG. 25 provides a diagram showing how uEEDs of the disclosure are tested for the delivery of tester molecules (e.g., GalNAc-siRNA-uEED, Antibody siRNA-uEED Conjugate (ARC), Lipid NanoParticle-uEED (LNP)).

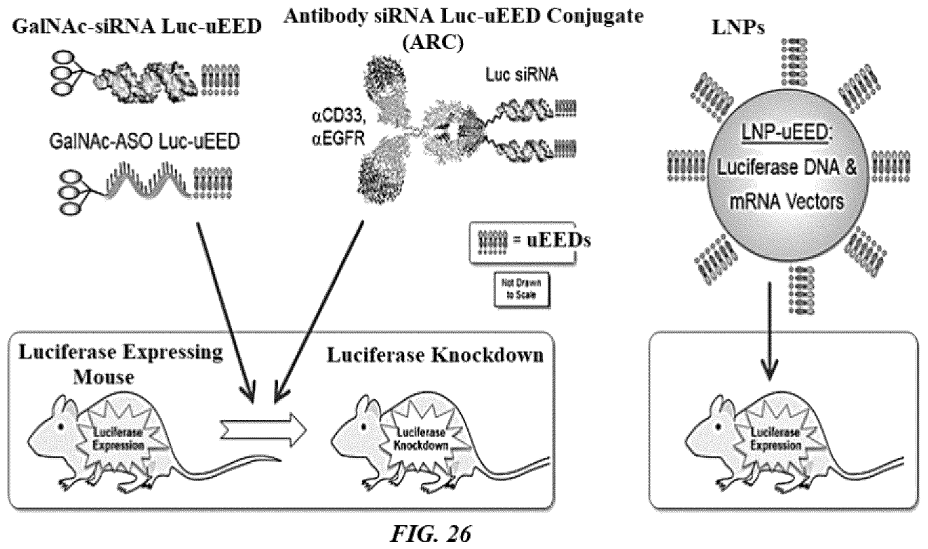

FIG. 26 provides a diagram showing how uEEDs of the disclosure are expected to knockdown or increase expression of luciferase in vivo by the delivery of tester molecules (e.g., GalNAc-siRNA-uEED, Antibody siRNA-uEED Conjugate (ARC), Lipid NanoParticle-uEED (LNP)).

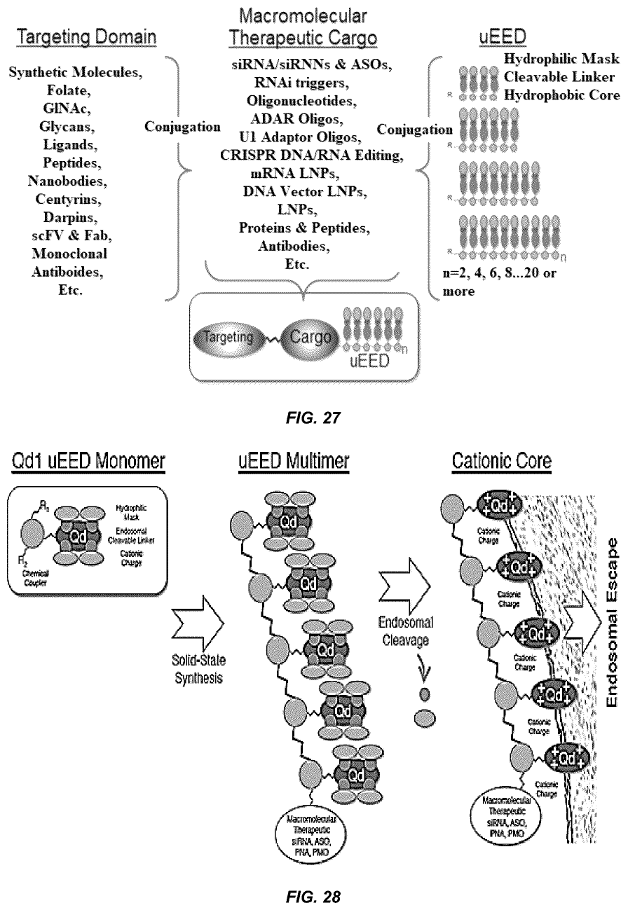

FIG. 27 shows a compositional structure of comprising a uEED of the disclosure having a targeting domain, conjugated to a cargo domain which is conjugated to a uEED domain.

FIG. 28 shows a schematic depicting a Qd-uEED monomer, multimer and mechanism of action. The Qd-uEED comprises cationic domains the promote endosomal escape.

FIG. 29 shows a schematic of a uEED multimer that comprises both Qd-uEED and Qa,b,c-uEED monomeric units.

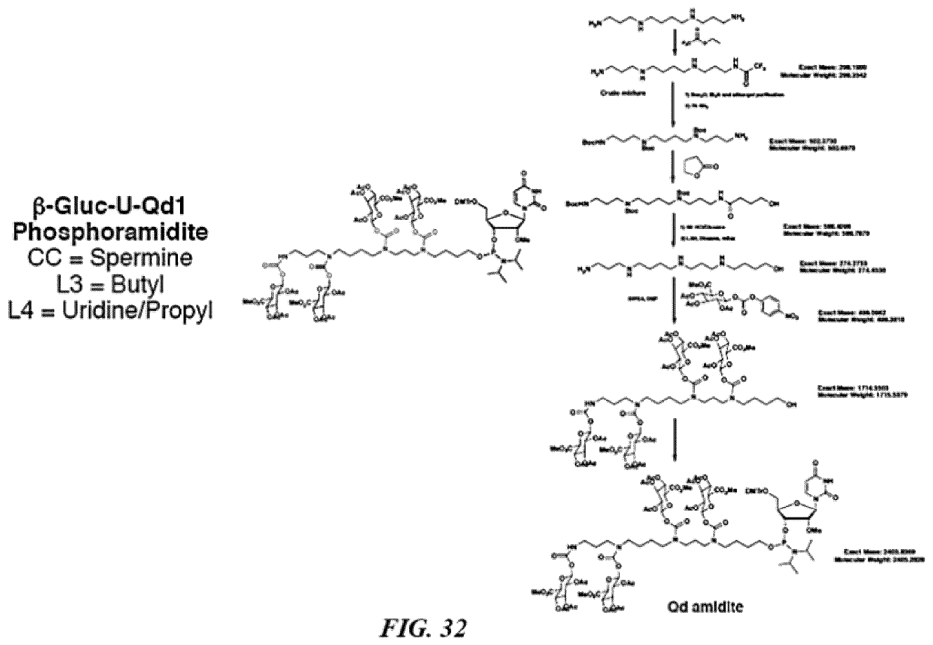

FIG. 30 shows a schematic of a Qd-s-uEED monomer and exemplary domains. Each monomeric unit can comprise a plurality of cationic charges.

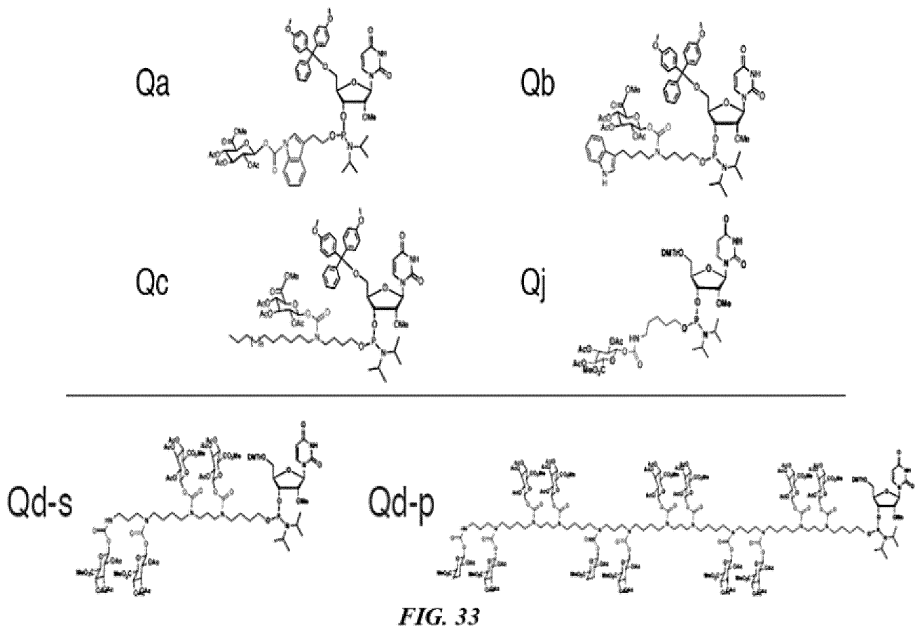

FIG. 31 shows examples of Qd-s and Qd-p uEED monomer phosphoramidites.

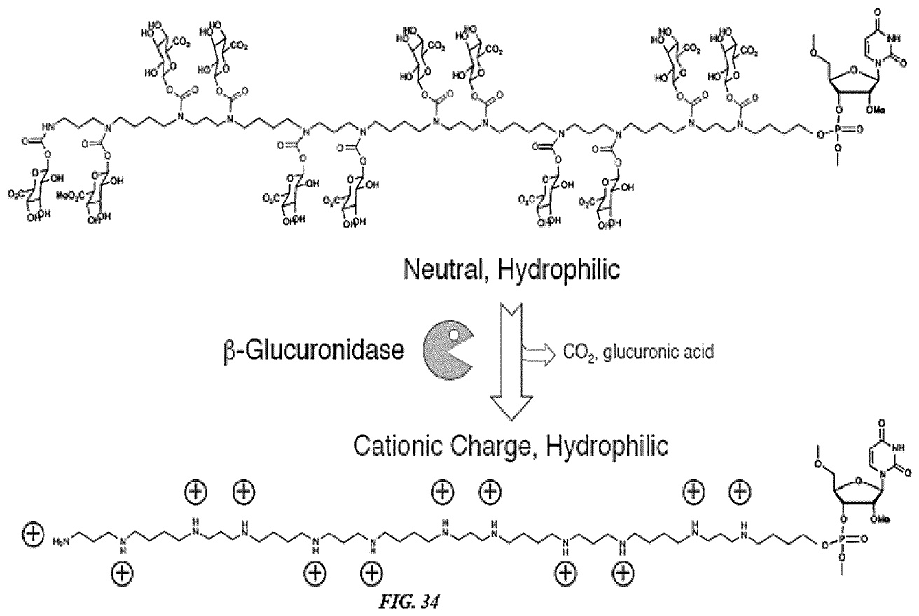

FIG. 32 shows an exemplary Qd-s uEED monomer synthesis.

FIG. 33 shows exemplary structures of Qa, Qb, Qc, Qj, Qd-s and Qd-p uEED monomer phosphoramidites.

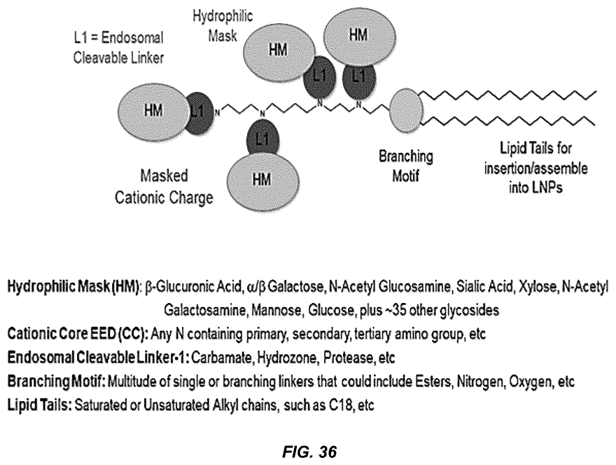

FIG. 34 shows an example of activation of Qd-p uEED by glucuronidase that results in exposure of multiple cationic charges.

FIG. 35 shows an example of activation of Qd-s uEED by glucuronidase that results in exposure of multiple cationic charges.

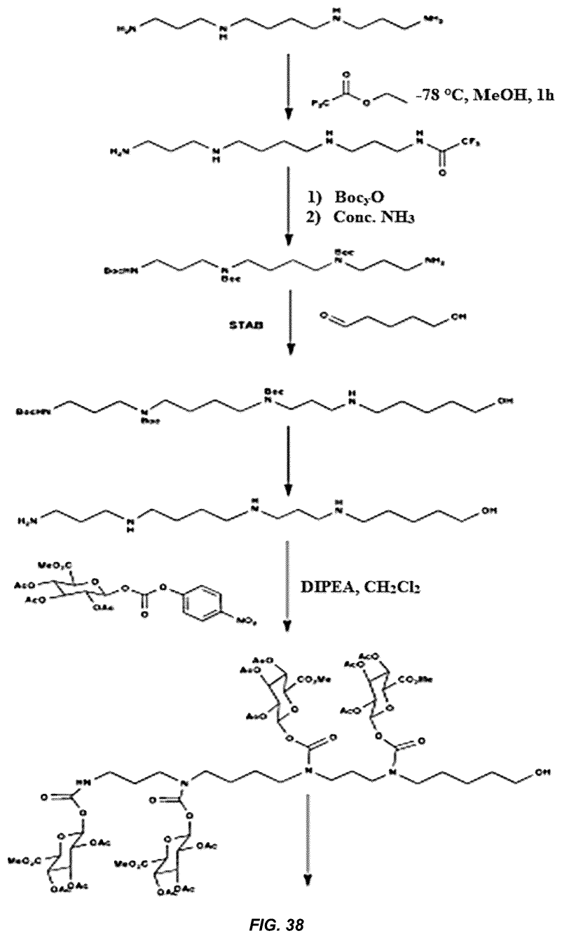

FIG. 36 provides a depiction of a Qf uEED construct of the disclosure. In this embodiment, a linker comprising, for example, esters, amino of oxygen entities are linked to a lipid tail(s) for insertion of assembly into lipid nanoparticles (LNP).

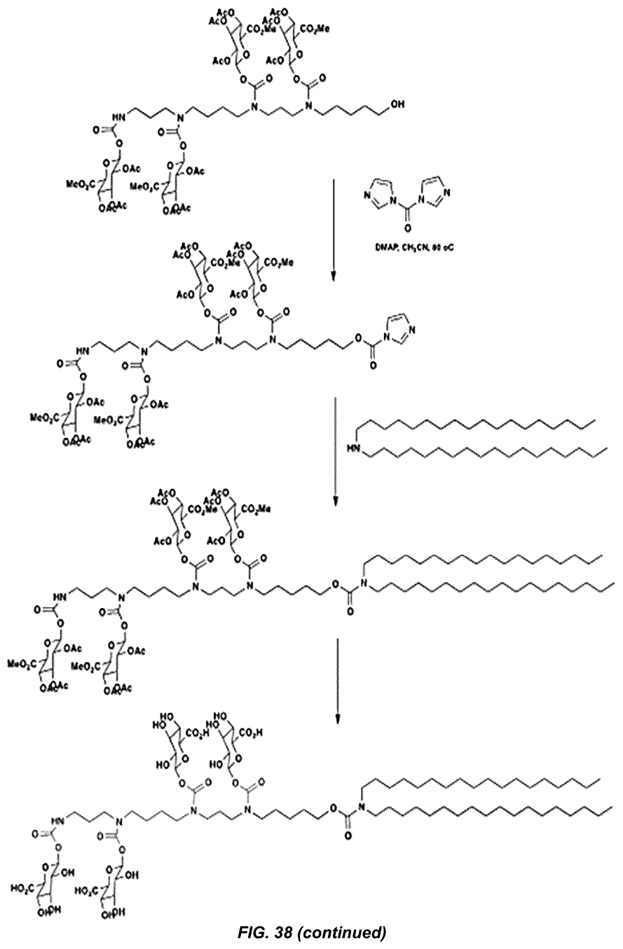

FIG. 37 shows activation of a Qf uEED of FIG. 36 via removal of the hydrophilic domain and exposure of the cationic charged domain.

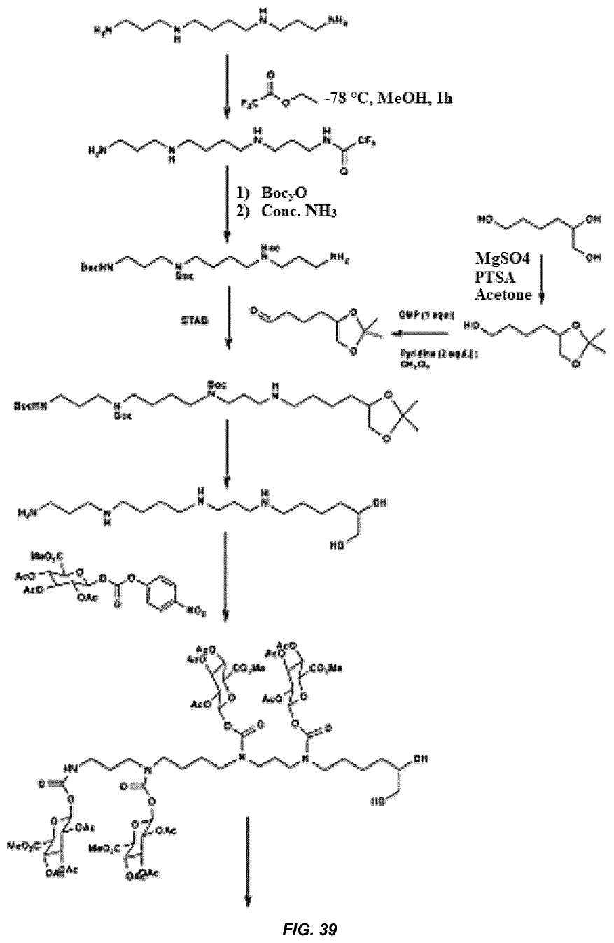

FIG. 38 shows a synthesis scheme of a Qf uEED of the disclosure.

FIG. 39 shows a synthesis scheme of a Qf uEED of the disclosure.

FIG. 40 shows a synthesis scheme of a Qf uEED of the disclosure.

FIG. 41 shows an SDS PAGE gel of various time incubation periods of siLuc linked to a Qb uEED in serum conditions.

FIG. 42 shows an SDS PAGE gel of various time periods of siLuc linked to Qb uEED in lysosomal conditions with beta-glucuronidase.

FIG. 43 shows an SDS PAGE gel of various time periods of siLuc linked to Qb uEED in lysosomal conditions with beta-glucuronidase.

FIG. 44A-C show Qe uEED embodiments of the disclosure. (A) shows a monomeric unit. (B) shows a multimer and schematic of endosomal activation. (C) shows a monomeric Qe uEED in more detail.

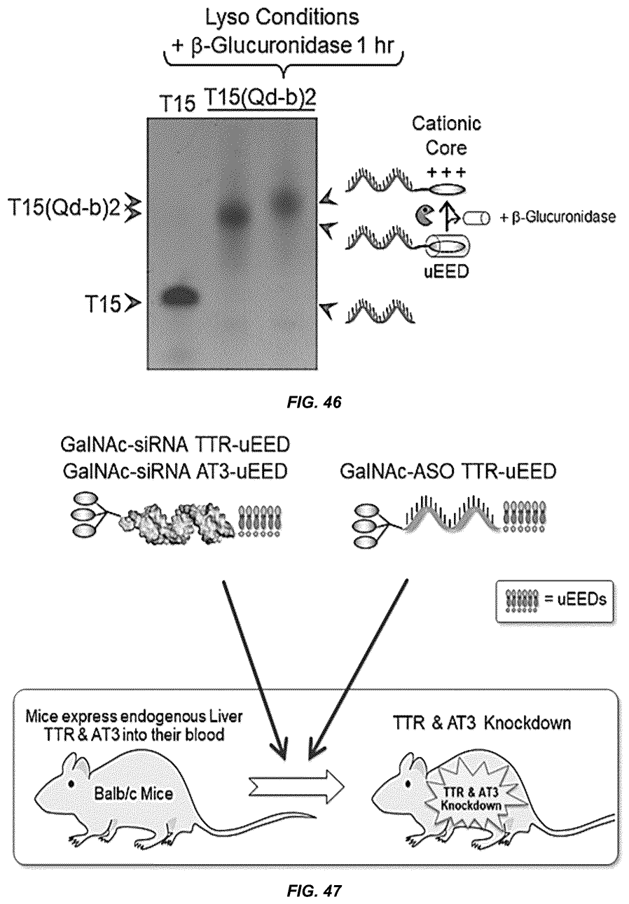

FIG. 45 shows a synthesis scheme for Qe uEED.

FIG. 46 shows the results of an oligo linked to a Qd uEED construct and treated with glucoronidase.

FIG. 47 provides a diagram of a mouse study using uEEDs of the disclosure.

FIG. 48 shows predicted results from a mouse study.

FIG. 49A-F provide (A) depiction of a Qa uEED construct and coupler backbones used in synthesis; (B) depiction of a Qb uEED construct and coupler backbones used in synthesis; (C) depiction of a Qc uEED construct and coupler backbones used in synthesis; (D) depiction of Qd uEED construct and coupler backbones used in synthesis; (E) depiction of a Qe uEED construct and coupler backbones used in synthesis; and (F) depiction of J uEED construct.

### DETAILED DESCRIPTION

As used herein and in the appended claims, the singular forms “a,” “an,” and “the” include plural referents unless the context clearly dictates otherwise. Thus, for example, reference to “a cargo” includes a plurality of such cargoes and reference to “the linker” includes reference to one or more linkers, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

The publications discussed above and throughout the text are provided solely for their disclosure prior to the filing date of the present application. Nothing herein is to be construed as an admission that the inventors are not entitled to antedate such disclosure by virtue of prior disclosure. Moreover, with respect to any term that is presented in one or more publications that is similar to, or identical with, a term that has been expressly defined in this disclosure, the definition of the term as expressly provided in this disclosure will control in all respects.

The term “alkenyl”, refers to an organic group that is comprised of carbon and hydrogen atoms that contains at least one double covalent bond between two carbons. Typically, an “alkenyl” as used in this disclosure, refers to organic group that contains 2, 3, 4, 5, 6, 7, 8, 9, 10, 11, 12, 13, 14, 15, 16, 17, 18, 19, 20, or 30 carbon atoms, or any range of carbon atoms between or including any two of the foregoing values. While a C₂-alkenyl can form a double bond to a carbon of a parent chain, an alkenyl group of three or more carbons can contain more than one double bond. It certain instances the alkenyl group will be conjugated, in other cases an alkenyl group will not be conjugated, and yet other cases the alkenyl group may have stretches of conjugation and stretches of nonconjugation. Additionally, if there is more than 2 carbon, the carbons may be connected in a linear manner, or alternatively if there are more than 3 carbons then the carbons may also be linked in a branched fashion so that the parent chain contains one or more secondary, tertiary, or quaternary carbons. An alkenyl may be substituted or unsubstituted, unless stated otherwise.

The term “alkyl”, refers to an organic group that is comprised of carbon and hydrogen atoms that contains single covalent bonds between carbons. Typically, an “alkyl” as used in this disclosure, refers to an organic group that contains 1, 2, 3, 4, 5, 6, 7, 8, 9, 10, 11, 12, 13, 14, 15, 16, 17, 18, 19, 20, or 30 carbon atoms, or any range of carbon atoms between or including any two of the foregoing values. Where if there is more than 1 carbon, the carbons may be connected in a linear manner, or alternatively if there are more than 2 carbons then the carbons may also be linked in a branched fashion so that the parent chain contains one or more secondary, tertiary, or quaternary carbons. An alkyl may be substituted or unsubstituted, unless stated otherwise.

The term “alkynyl”, refers to an organic group that is comprised of carbon and hydrogen atoms that contains a triple covalent bond between two carbons. Typically, an “alkynyl” as used in this disclosure, refers to organic group that contains that contains 2, 3, 4, 5, 6, 7, 8, 9, 10, 11, 12, 13, 14, 15, 16, 17, 18, 19, 20, or 30 carbon atoms, or any range of carbon atoms between or including any two of the foregoing values. While a C2-alkynyl can form a triple bond to a carbon of a parent chain, an alkynyl group of three or more carbons can contain more than one triple bond. Where if there is more than 3 carbon, the carbons may be connected in a linear manner, or alternatively if there are more than 4 carbons then the carbons may also be linked in a branched fashion so that the parent chain contains one or more secondary, tertiary, or quaternary carbons. An alkynyl may be substituted or unsubstituted, unless stated otherwise.

The term “amino,” as used herein, represents —N(R^{N1})₂ or —N(═NR^{N1})(NR^{N1})₂ wherein each R^{N1} is, independently, H, OH, NO₂, N(R^{N2})₂, SO₂OR^{N2}, SO₂R^{N2}, SOR^{N2}, an N-protecting group, alkyl, alkenyl, alkynyl, alkoxy, aryl, alkaryl, cycloalkyl, alkcycloalkyl, heterocyclyl (e.g., heteroaryl), alkheterocyclyl (e.g., alkheteroaryl), or two R^{N1} combine to form a heterocyclyl, and wherein each R^{N2} is, independently, H, alkyl, or aryl. In one embodiment, amino is —NH₂, or —NHR^{N1}, wherein R^{N1} is, independently, OH, NO₂, NH₂, NR^{N2}₂, SO₂OR^{N2}, SO₂R^{N2}, SOR^{N2}, alkyl, or aryl, and each R^{N2} can be H, alkyl, or aryl. The R^{N1} groups may themselves be unsubstituted or substituted as described herein.

The term “aryl”, as used in this disclosure, refers to a conjugated planar ring system with delocalized pi electron clouds that contain only carbon as ring atoms. An “aryl” for the purposes of this disclosure encompass from 1 to 4 aryl rings wherein when the aryl is greater than 1 ring the aryl rings are joined so that they are linked, fused, or a combination thereof. An aryl may be substituted or unsubstituted, or in the case of more than one aryl ring, one or more rings may be unsubstituted, one or more rings may be substituted, or a combination thereof.

The term generally represented by the notation “Cₓ-C_{y}” (where x and y are whole integers and y>x) prior to a functional group, e.g., “C₁-C₁₂ alkyl” refers to a number range of carbon atoms. For the purposes of this disclosure any range specified by “Cₓ-C_{y}” (where x and y are whole integers and y>x) is not exclusive to the expressed range, but is inclusive of all possible ranges that include and fall within the range specified by “Cₓ-C_{y}” (where x and y are whole integers and y>x). For example, the term “C₁-C₄” provides express support for a range of 1 to 4 carbon atoms, but further provides implicit support for ranges encompassed by 1 to 4 carbon atoms, such as 1 to 2 carbon atoms, 1 to 3 carbon atoms, 2 to 3 carbon atoms, 2 to 4 carbon atoms, and 3 to 4 carbon atoms.

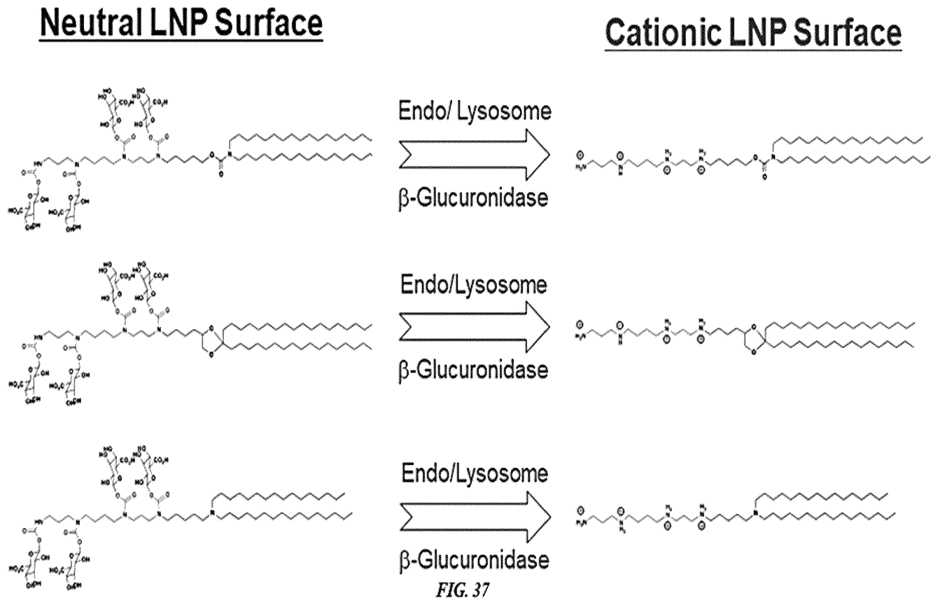

Cationic domains include protein transduction domains (PTDs; sometimes referred to as cell penetrating peptides (CPPs)), guanidinium groups, primary amines, secondary amines, tertiary amines, complex amino groups, and ionizable amines. In one embodiment, a cationic domain (cationic charge domain) can comprise multiple cationic charges (e.g., 1-10, 11-20, 21-50 or more) on a single unit structure (see, e.g., FIG. 34). Examples of positively charged polymers include poly(ethylene imine) (PEI), spermine, spermidine, and poly(amidoamine) (PAMAM).

Several cationic lipids have been described in the literature, many of which are commercially available. In some embodiments, the cationic lipid N-[1-(2,3-dioleyloxy)propyl]-N,N,N-trimethylammonium chloride or “DOTMA” is used. (Felgner et al. (Proc. Nat'l Acad. Sci. 84, 7413 (1987); U.S. Pat. No. 4,897,355). Other suitable cationic lipids include, for example, ionizable cationic lipids, such as, e.g., (15Z,18Z)—N,N-dimethyl-6-(9Z,12Z)-octadeca-9,12-dien-1-yl)tetracosa-15,18-dien-1-amine (HGT5000), (15Z,18Z)—N,N-dimethyl-6-((9Z,12Z)-octadeca-9,12-dien-1-yl)tetracosa-4,15-,18-trien-1-amine (HGT5001), and (15Z,18Z)—N,N-dimethyl-6-((9Z,12Z)-octadeca-9,12-dien-1-yl)tetracosa-5,15-,18-trien-1-amine (HGT5002); C12-200 (WO 2010/053572), 2-(2,2-di((9Z,12Z)-octadeca-9,12-dien-1-yl)-1,3-dioxolan-4-yl)-N,N-dimeth-ylethanamine (DLinKC2-DMA)), 2-(2,2-di((9Z,12Z)-octadeca-9,12-dien-1-yl)-1,3-dioxolan-4-yl)-N,N-dimeth-ylethanamine “DLin-KC2-DMA,” (3 S,10R,13R,17R)-10,13-dimethyl-17-((R)-6-methylheptan-2-yl)-2,3,4,7,8,9,-10,11,12,13,14,15,16,17-tetradecahydro-1H-cyclopenta[a]phenanthren-3-yl 3-(1H-imidazol-4-yl)propanoate “ICE,” (15Z,18Z)—N,N-dimethyl-6-(9Z,12Z)-octadeca-9,12-dien-1-yl)tetracosa-15,18-dien-1-amine “HGT5000,” (15Z,18Z)—N,N-dimethyl-6-((9Z,12Z)-octadeca-9,12-dien-1-yl)tetracosa-4,15-,18-trien-1-amine “HGT5001,” and (15Z,18Z)—N,N-dimethyl-6-((9Z,12Z)-octadeca-9,12-dien-1-yl)tetracosa-5,15-,18-trien-1-amine “HGT5002,” 5-carboxyspermylglycine-dioctadecylamide or “DOGS,” 2,3-dioleyloxy-N-[2(spermine-carboxamido)ethyl]-N,N-dimethyl-1-pr-opanaminium or “DOSPA”, 1,2-Dioleoyl-3-Dimethylammonium-Propane or “DODAP”, 1,2-Dioleoyl-3-Trimethylammonium-Propane or “DOTAP”. Contemplated cationic lipids also include 1,2-distearyloxy-N,N-dimethyl-3-aminopropane or “DSDMA”, 1,2-dioleyloxy-N,N-dimethyl-3-aminopropane or “DODMA”, 1,2-dilinoleyloxy-N,N-dimethyl-3-aminopropane or “DLinDMA”, 1,2-dilinolenyloxy-N,N-dimethyl-3-aminopropane or “DLenDMA”, N-dioleyl-N,N-dimethylammonium chloride or “DODAC”, N,N-distearyl-N,N-dimethylammonium bromide or “DDAB”, N-(1,2-dimyristyloxyprop-3-yl)-N,N-dimethyl-N-hydroxyethyl ammonium bromide or “DMRIE”, 3-dimethylamino-2-(cholest-5-en-3-beta-oxybutan-4-oxy)-1-(cis,cis-9,12-oc-tadecadienoxy)propane or “CLinDMA”, 2-[5′-(cholest-5-en-3-beta-oxy)-3-oxapentoxy)-3-dimethyl-1-(cis,cis-9′,1-2′-octadecadienoxy)propane or “CpLinDMA”, N,N-dimethyl-3,4-dioleyloxybenzylamine or “DMOBA”, 1,2-N,N′-dioleylcarbamyl-3-dimethylaminopropane or “DOcarbDAP”, 2,3-Dilinoleoyloxy-N,N-dimethylpropylamine or “DLinDAP”, 1,2-N,N′-Dilinoleylcarbamyl-3-dimethylaminopropane or “DLincarbDAP”, 1,2-Dilinoleoylcarbamyl-3-dimethylaminopropane or “DLinCDAP”, 2,2-dilinoleyl-4-dimethylaminomethyl-[1,3]-dioxolane or “DLin-K-DMA”, 2,2-dilinoleyl-4-dimethylaminoethyl[1,3]-dioxolane or “DLin-K-XTC2-DMA”, or mixtures thereof. Suitable cholesterol-based cationic lipids include, for example, DC-Chol (N,N-dimethyl-N-ethylcarboxamidocholesterol), 1,4-bis(3-N-oleylamino-propyl)piperazine.

Also contemplated are cationic lipids such as the dialkylamino-based, imidazole-based, and guanidinium-based lipids. For example, also contemplated is the use of the cationic lipid (3S,10R,13R,17R)-10,13-dimethyl-17-((R)-6-methylheptan-2-yl)-2,3,4,7,8,9,-10,11,12,13,14,15,16,17-tetradecahydro-1H-cyclopenta[a]phenanthren-3-yl 3-(1H-imidazol-4-yl)propanoate or “ICE”.

The term “cycloalkenyl”, as used in this disclosure, refers to an alkene that contains at least 4 carbon atoms but no more than 12 carbon atoms connected so that it forms a ring. A “cycloalkenyl” for the purposes of this disclosure encompasses from 1 to 4 cycloalkenyl rings, wherein when the cycloalkenyl is greater than 1 ring, then the cycloalkenyl rings are joined so that they are linked, fused, or a combination thereof. A cycloalkenyl may be substituted or unsubstituted, or in the case of more than one cycloalkenyl ring, one or more rings may be unsubstituted, one or more rings may be substituted, or a combination thereof.

The term “cycloalkyl”, as used in this disclosure, refers to an alkyl that contains at least 3 carbon atoms but no more than 12 carbon atoms connected so that it forms a ring. A “cycloalkyl” for the purposes of this disclosure encompasses from 1 to 4 cycloalkyl rings, wherein when the cycloalkyl is greater than 1 ring, then the cycloalkyl rings are joined so that they are linked, fused, or a combination thereof. A cycloalkyl may be substituted or unsubstituted, or in the case of more than one cycloalkyl ring, one or more rings may be unsubstituted, one or more rings may be substituted, or a combination thereof.

The term “disorder” as used herein is intended to be generally synonymous, and is used interchangeably with, the terms “disease,” “syndrome,” and “condition” (as in medical condition), in that all reflect an abnormal condition of the human or animal body or of one of its parts that impairs normal functioning, is typically manifested by distinguishing signs and symptoms.

The term “endosomal escape moiety,” as used herein, represents a moiety which enhances the release of endosomal contents or allows for the escape of a molecule from an internal cellular compartment such as an endosome. An endosomal escape moiety generally destabilizes an endosomal or lysosomal membrane. In certain embodiments, the endosomal escape moiety is a hydrophobic domain or a cationic domain.

The term “glycoside” refers to a molecule in which a sugar group is bonded through its anomeric carbon to another group via a glycosidic bond. Glycosides can be linked by an O- (an O-glycoside), N- (a glycosylamine), S- (a thioglycoside), or C- (a C-glycoside) glycosidic bond. An empirical formula is Cₘ(H₂O)ₙ (where m can be different from n, and m and n are <36), Glycoside herein includes glucose (dextrose), fructose (levulose) allose, altrose, mannose, gulose, isodose, galactose, talose, galactosamine, glucosamine, sialic acid, N-acetylglucosamine, sulfoquinovose (6-deoxy-6-sulfo-D-glucopyranose), ribose, arabinose, xylose, lyxose, sorbitol, mannitol, sucrose, lactose, maltose, trehalose, maltodextrins, raffinose, Glucuronic acid (glucuronide), and stachyose. It can be in D form or L form, 5 atoms cyclic furanose forms, 6 atoms cyclic pyranose forms, or acyclic form, α-isomer (the —OH of the anomeric carbon below the plane of the carbon atoms of Haworth projection), or a β-isomer (the —OH of the anomeric carbon above the plane of Haworth projection). It is used herein as a monosaccharide, disaccharide, polyols, or oligosaccharides containing 3-6 sugar units. Of particular use in the compositions and methods of the disclosure are glycosides that can be cleaved by endosomal glycosidases. Glycosidases (sometimes referred to as glycoside hydrolases) are known enzymes that hydrolyze glycosidic bonds. Glycosidases are classified in EC 3.2.1 as enzymes that catalyze the hydrolysis of O- or S-glycosides.

The term “hetero-” when used as a prefix, such as, hetero-alkyl, hetero-alkenyl, hetero-alkynyl, or hetero-hydrocarbon, for the purpose of this disclosure refers to the specified hydrocarbon having one or more carbon atoms replaced by non-carbon atoms as part of the parent chain. Examples of such non-carbon atoms include, but are not limited to, N, O, S, Si, Al, B, and P. If there is more than one non-carbon atom in the hetero-based parent chain then this atom may be the same element or may be a combination of different elements, such as N and O. In a particular embodiment, a “hetero”-hydrocarbon (e.g., alkyl, alkenyl, alkynyl) refers to a hydrocarbon that has from 1 to 3 C, N and/or S atoms as part of the parent chain.

The term “heterocycle,” as used herein, refers to ring structures that contain at least 1 noncarbon ring atom. A “heterocycle” for the purposes of this disclosure encompass from 1 to 4 heterocycle rings, wherein when the heterocycle is greater than 1 ring the heterocycle rings are joined so that they are linked, fused, or a combination thereof. A heterocycle may be aromatic or nonaromatic, or in the case of more than one heterocycle ring, one or more rings may be nonaromatic, one or more rings may be aromatic, or a combination thereof. A heterocycle may be substituted or unsubstituted, or in the case of more than one heterocycle ring one or more rings may be unsubstituted, one or more rings may be substituted, or a combination thereof. Typically, the noncarbon ring atom is N, O, S, Si, Al, B, or P. In the case where there is more than one noncarbon ring atom, these noncarbon ring atoms can either be the same element, or combination of different elements, such as N and O. Examples of heterocycles include, but are not limited to: a monocyclic heterocycle such as, aziridine, oxirane, thiirane, azetidine, oxetane, thietane, pyrrolidine, pyrroline, imidazolidine, pyrazolidine, pyrazoline, dioxolane, sulfolane 2,3-dihydrofuran, 2,5-dihydrofuran tetrahydrofuran, thiophane, piperidine, 1,2,3,6-tetrahydro-pyridine, piperazine, morpholine, thiomorpholine, pyran, thiopyran, 2,3-dihydropyran, tetrahydropyran, 1,4-dihydropyridine, 1,4-dioxane, 1,3-dioxane, dioxane, homopiperidine, 2,3,4,7-tetrahydro-1H-azepine homopiperazine, 1,3-dioxepane, 4,7-dihydro-1,3-dioxepin, and hexamethylene oxide; and polycyclic heterocycles such as, indole, indoline, isoindoline, quinoline, tetrahydroquinoline, isoquinoline, tetrahydroisoquinoline, 1,4-benzodioxan, coumarin, dihydrocoumarin, benzofuran, 2,3-dihydrobenzofuran, isobenzofuran, chromene, chroman, isochroman, xanthene, phenoxathiin, thianthrene, indolizine, isoindole, indazole, purine, phthalazine, naphthyridine, quinoxaline, quinazoline, cinnoline, pteridine, phenanthridine, perimidine, phenanthroline, phenazine, phenothiazine, phenoxazine, 1,2-benzisoxazole, benzothiophene, benzoxazole, benzthiazole, benzimidazole, benztriazole, thioxanthine, carbazole, carboline, acridine, pyrolizidine, and quinolizidine. In addition to the polycyclic heterocycles described above, heterocycle includes polycyclic heterocycles wherein the ring fusion between two or more rings includes more than one bond common to both rings and more than two atoms common to both rings. Examples of such bridged heterocycles include quinuclidine, diazabicyclo[2.2.1]heptane and 7-oxabicyclo[2.2.1]heptane.

The terms “heterocyclic group”, “heterocyclic moiety”, “heterocyclic”, or “heterocyclo” used alone or as a suffix or prefix, refers to a heterocycle that has had one or more hydrogens removed therefrom.

The term “hydrocarbons” refers to groups of atoms that contain only carbon and hydrogen. Examples of hydrocarbons that can be used in this disclosure include, but are not limited to, alkanes, alkenes, alkynes, arenes, and benzyls.

The term “hydrophilic group,” or “hydrophilic domain,” as used herein, represents a moiety or domain that confers an affinity to water and increase the solubility of an construct in water. Hydrophilic groups can be ionic or non-ionic and include moieties that are positively charged, negatively charged, and/or can engage in hydrogen-bonding interactions.

The term “non-release controlling excipient” as used herein, refers to an excipient whose primary function do not include modifying the duration or place of release of the active substance from a dosage form as compared with a conventional immediate release dosage form.

The term “optionally substituted” refers to a functional group, typically a hydrocarbon or heterocycle, where one or more hydrogen atoms may be replaced with a substituent. Accordingly, “optionally substituted” refers to a functional group that is substituted, in that one or more hydrogen atoms are replaced with a substituent, or unsubstituted, in that the hydrogen atoms are not replaced with a substituent. For example, an optionally substituted hydrocarbon group refers to an unsubstituted hydrocarbon group or a substituted hydrocarbon group.

The term “peptide,” as used herein, represents two to about 50 amino acid residues linked by peptide bonds. The term “polypeptide,” as used herein, represents chains of 50 or more amino acids linked by peptide bonds. Moreover, for purposes of this disclosure, the term “polypeptide” and the term “protein” are used interchangeably herein in all contexts, unless provided for otherwise, e.g., naturally occurring or engineered proteins. A variety of polypeptides may be used within the scope of the methods and compositions provided herein. In a certain embodiment, polypeptides include antibodies or fragments of antibodies containing an antigen-binding site. In other embodiments, a polypeptide can include enzymatically active entities (e.g., Cas protein) and the like. Polypeptides made synthetically may include substitutions of amino acids not naturally encoded by DNA (e.g., non-naturally occurring or unnatural amino acid). Examples of non-naturally occurring amino acids include D-amino acids, an amino acid having an acetylaminomethyl group attached to a sulfur atom of a cysteine, a pegylated amino acid, the omega amino acids of the formula NH₂(CH₂)ₙCOOH wherein n is 2-6, neutral nonpolar amino acids, such as sarcosine, t-butyl alanine, t-butyl glycine, N-methyl isoleucine, and norleucine.

The term “pharmaceutically acceptable carrier,” “pharmaceutically acceptable excipient,” “physiologically acceptable carrier,” or “physiologically acceptable excipient” as used herein, refers to a pharmaceutically-acceptable material, composition, or vehicle, such as a liquid or solid filler, diluent, excipient, solvent, or encapsulating material. Each component must be “pharmaceutically acceptable” in the sense of being compatible with the other ingredients of a pharmaceutical formulation. It must also be suitable for use in contact with the tissue or organ of humans and animals without excessive toxicity, irritation, allergic response, immunogenicity, or other problems or complications, commensurate with a reasonable benefit/risk ratio. Examples of “pharmaceutically acceptable carriers” and “pharmaceutically acceptable excipients” can be found in the following, Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004.

The term “polynucleotide” or “nucleic acid” as used herein, represents two or more nucleotides and/or nucleosides covalently bound together by an internucleotide bridging group. Polynucleotides may be linear or circular. Moreover, for the purposes of this disclosure, the term “polynucleotide” is in reference to both oligonucleotides and longer sequences, and to mixtures of nucleotides, e.g., mixtures of DNA and RNA or mixtures of RNA and 2′ modified RNA. The term “polynucleotide” encompasses polynucleotides which are comprised of one or more strands, unless stated otherwise. The term polynucleotide includes DNA, RNA, including double stranded and single stranded forms thereof, DNA/RNA hybrids and the like.

The term “protecting group,” as used herein, represents a group intended to protect a functional group (e.g., a hydroxyl, an amino, or a carbonyl) from participating in one or more undesirable reactions during chemical synthesis (e.g., polynucleotide synthesis). The term “O-protecting group,” as used herein, represents a group intended to protect an oxygen containing (e.g., phenol, hydroxyl or carbonyl) group from participating in one or more undesirable reactions during chemical synthesis. The term “N-protecting group,” as used herein, represents a group intended to protect a nitrogen containing (e.g., an amino or hydrazine) group from participating in one or more undesirable reactions during chemical synthesis. Commonly used O- and N-protecting groups are disclosed in Greene, “Protective Groups in Organic Synthesis,” 3^{rd} Edition (John Wiley & Sons, New York, 1999), which is incorporated herein by reference. Exemplary O- and N-protecting groups include acyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, t-butyldimethylsilyl, tri-iso-propylsilyloxymethyl, 4,4′-dimethoxytrityl, isobutyryl, phenoxyacetyl, 4-isopropylpehenoxyacetyl, dimethylformamidino, and 4-nitrobenzoyl.

Exemplary O-protecting groups for protecting carbonyl containing groups include, but are not limited to: acetals, acylals, 1,3-dithianes, 1,3-dioxanes, 1,3-dioxolanes, and 1,3-dithiolanes.

Other O-protecting groups include, but are not limited to: substituted alkyl, aryl, and alkaryl ethers (e.g., trityl; methylthiomethyl; methoxymethyl; benzyloxymethyl; siloxymethyl; 2,2,2,-trichloroethoxymethyl; tetrahydropyranyl; tetrahydrofuranyl; ethoxyethyl; 1-[2-(trimethylsilyl)ethoxy]ethyl; 2-trimethylsilylethyl; t-butyl ether; p-chlorophenyl, p-methoxyphenyl, p-nitrophenyl, benzyl, p-methoxybenzyl, and nitrobenzyl); silyl ethers (e.g., trimethylsilyl; triethylsilyl; triisopropylsilyl; dimethylisopropylsilyl; t-butyldimethylsilyl; t-butyldiphenylsilyl; tribenzylsilyl; triphenylsilyl; and diphenylmethylsilyl); carbonates (e.g., methyl, methoxymethyl, 9-fluorenylmethyl; ethyl; 2,2,2-trichloroethyl; 2-(trimethylsilyl)ethyl; vinyl, allyl, nitrophenyl; benzyl; methoxybenzyl; 3,4-dimethoxybenzyl; and nitrobenzyl).

Other N-protecting groups include, but are not limited to, chiral auxiliaries such as protected or unprotected D, L or D, L-amino acids such as alanine, leucine, phenylalanine, and the like; sulfonyl-containing groups such as benzenesulfonyl, p-toluenesulfonyl, and the like; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyl oxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, phenylthiocarbonyl, and the like, alkaryl groups such as benzyl, triphenylmethyl, benzyloxymethyl, and the like and silyl groups such as trimethylsilyl, and the like. Useful N-protecting groups are formyl, acetyl, benzoyl, pivaloyl, t-butylacetyl, alanyl, phenylsulfonyl, benzyl, t-butyloxycarbonyl (Boc), and benzyloxycarbonyl (Cbz).

The term “release controlling excipient” as used herein, refers to an excipient whose primary function is to modify the duration or place of release of the active substance from a dosage form as compared with a conventional immediate release dosage form.

The term “subject” as used herein, refers to an animal, including, but not limited to, a primate (e.g., human, monkey, chimpanzee, gorilla, and the like), rodents (e.g., rats, mice, gerbils, hamsters, ferrets, and the like), lagomorphs, swine (e.g., pig, miniature pig), equine, canine, feline, and the like. The terms “subject” and “patient” are used interchangeably herein. For example, a mammalian subject can refer to a human subject or patient.

The term “substituent” refers to an atom or group of atoms substituted in place of a hydrogen atom. For purposes of this invention, a substituent would include deuterium atoms.

The term “substituted” with respect to hydrocarbons, heterocycles, and the like, refers to structures wherein the parent chain contains one or more substituents.

The term “targeting moiety,” as used herein, represents any moiety that specifically binds or reactively associates or complexes with a receptor or other receptive moiety associated with a given target cell population or a moiety that induces endocytosis when contact with a cell or is endocytosed by a cell.

The term “therapeutically acceptable” refers to those compounds (or salts, prodrugs, tautomers, zwitterionic forms, etc.) which are suitable for use in contact with the tissues of patients without excessive toxicity, irritation, allergic response, immunogenicity, are commensurate with a reasonable benefit/risk ratio, and are effective for their intended use.

The terms “treat”, “treating” and “treatment”, as used herein, refers to ameliorating symptoms associated with a disease or disorder (e.g., multiple sclerosis), including preventing or delaying the onset of the disease or disorder symptoms, and/or lessening the severity or frequency of symptoms of the disease or disorder.

The term “unsubstituted” with respect to hydrocarbons, heterocycles, and the like, refers to structures wherein the parent chain contains no substituents.

The ability to deliver functional agents to cells is problematical due to the bioavailability restriction imposed by the cell membrane. That is, the plasma lipid bilayer membrane of the cell forms an effective barrier, which restricts the intracellular uptake of molecules to those which are sufficiently non-polar and smaller than approximately 500 Daltons in size. Previous efforts to enhance the internalization of proteins have focused on fusing proteins with receptor ligands (Ng et al., Proc. Natl. Acad. Sci. USA, 99:10706-11, 2002) or by packaging them into caged liposomal carriers (Abu-Amer et al., J. Biol. Chem. 276:30499-503, 2001). However, these techniques often result in poor cellular uptake and intracellular sequestration into the endocytic pathway. In addition, liposomal formulations can be cytotoxic.

All intracellular macromolecular therapeutics (including: siRNAs, ASOs, Peptides, Proteins, Large Synthetic Molecules, CRISPR, RNPs, mRNA, RNAs, DNA vector, LNPs, NPs, etc.) are taken up by cells by various forms of endocytosis. Endosomes comprise a lipid bilayer membrane barrier that prevents >99% of macromolecular therapeutic from escaping the endosome into the cytoplasm and nucleus of cells. Thus, endosomal escape remains a significant technological problem that needs to be solved to enable deliver of all macromolecular therapeutics. Enveloped viruses also have to address the endosomal escape problem and use a protein machine that contains an outer hydrophilic mask covering an inner hydrophobic endosomal escape domain.

PTDs/CPPs have been used to deliver therapeutic cargo into cells in culture, studied in pre-clinical models of disease and are currently in clinical trials. There are over 100 published PTD/CPP delivery domain sequences; however, most published PTDs/CPPs have been investigated using dye-labeled molecules. Consequently, excluding cell death, there is a paucity of quantitative transduction assays that rely on robust and well-controlled cellular phenotypes that can be readily quantified to determine which PTDs/CPPs are the most efficient and least cytotoxic delivery domains. Briefly, PTD/CPP delivery of macromolecules into the cytoplasm requires: (1) cellular association and uptake by endocytosis, and (2) escape from the endosome into the cytoplasm, which is the rate-limiting delivery step.

As mentioned above, even with effective uptake, escape from endosomes remains the rate-limiting step for delivery of macromolecular cargo into the cytoplasm by all delivery agents, including PTDs/CPPs and LNPs. It is estimated that only a small fraction of the endosomal bound (cell associated) TAT-PTD/CPP escapes from the macropinosome into the cytoplasm, perhaps as little as or even less than 1%. The disclosure provides endosomal escape domains having compositions that improve escape from the endosome of transported cargo across the endosomal lipid bilayer membrane into the cytoplasm and nucleus of the cell.

The disclosure provides compounds to solve the endosomal escape problem mimicking the viral escape mechanism. The synthetic constructs of the disclosure comprise an outer hydrophilic mask domain that is connected though an endosomal specific cleavable linker to a synthetic hydrophobic core and/or cationic endosomal escape domain. The compounds described herein are sometimes referred to as a Universal Endosomal Escape Domain (uEED) of which there are variations in their domains and arrangements thereof as set forth herein.

The disclosure provides a universal Endosomal Escape Domain (uEED) composition comprises a hydrophilic mask domain linked to a cleavable linker and a cationic and/or hydrophobic core linked to a cargo molecule wherein the cleavable linker separates the hydrophilic mask from the cationic mask domain or hydrophobic domain. The cationic domain or hydrophobic domain can then interact with the endosomal membrane and destabilize the membrane to allow release of the cargo into the cytoplasm. The compounds of the disclosure promote uptake and release of macromolecules.

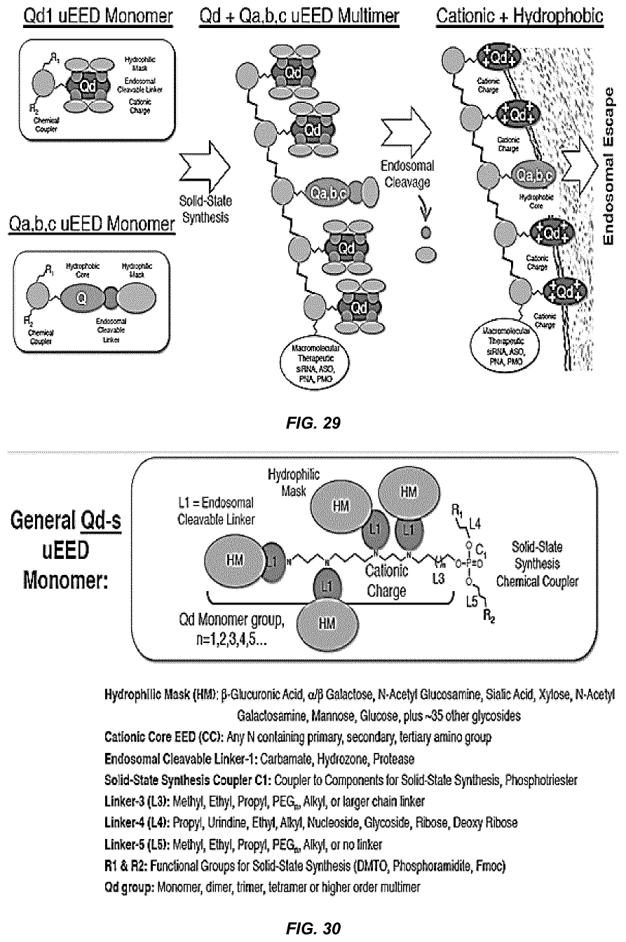

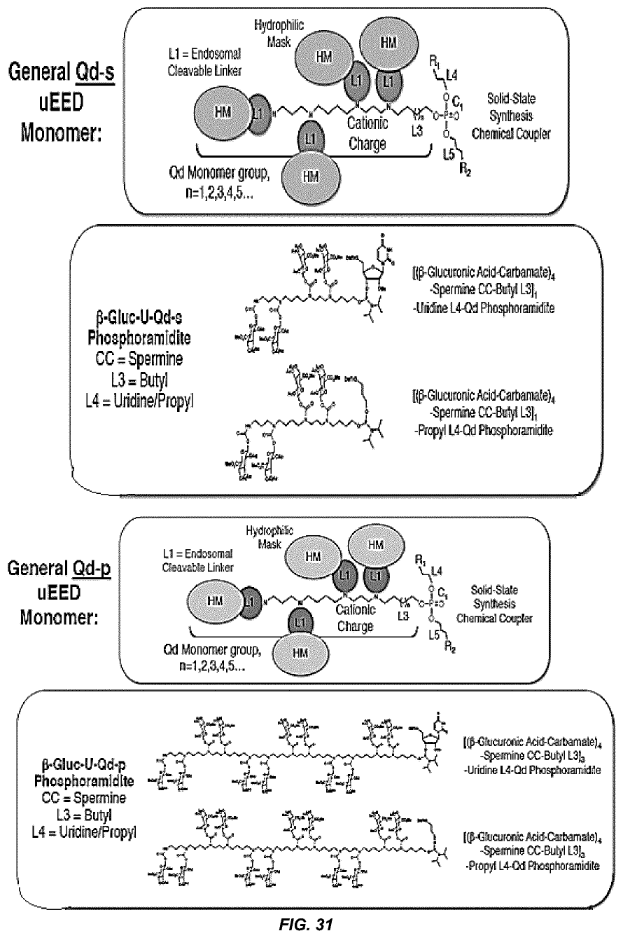

In the methods and compositions of the disclosure, a macromolecular cargo linked to a uEED of the disclosure is taken up by micropinocytosis/endocytosis via a targeting domain that either induces endocytosis or attaches to a receptor that undergoes endocytosis. Once taken up and present inside the endosome, the cleavable linker of the uEED is cleaved inside the endosome/lysosome to release the hydrophilic domain from the hydrophobic domain or cationic domain. The hydrophobic domain or cationic domain then inserts into and destabilizes the endosomal lipid bilayer membrane thereby releasing the cargo intracellularly. FIGS. 27 and 28 provides exemplary structures comprising a uEED of the disclosure.

The disclosure provides compounds useful in cellular transduction and cellular modulation. The cargo can be any number of different molecular entities including diagnostic and therapeutics for the treatment of a disease or disorder including small molecule and biologics for disease treatment. In one embodiment, the multi-domain approach can be used to deliver anticancer agents to a tumor cell and thereby kill tumor cells. The anti-cancer agent can be a peptide, polypeptide, protein, small molecule agent or an inhibitory nucleic acid (e.g., siRNA, ASO, oligonucleotide, ribozyme etc). In another embodiment, a macromolecular cargo can be delivered to a cell or tissue. Examples of macromolecular cargo including CRISPR/Cas systems, gRNA, adenosine deaminase acting on RNA (ADAR) and the like.

The disclosure provides compounds that comprise modular components that are operably linked such that each “component” or “domain” can serve a desired biological function. For example, a compound comprises a linker and/or coupler domain linked to a hydrophobic and/or cationic domain, wherein the hydrophobic and/or cationic domain is linked to a hydrophilic domain via a cleavable linker. Each module, e.g., the linker and/or coupler, the hydrophilic domain, the cleavable linker and the hydrophobic or cationic domain are functional for a particular purpose of releasing a cargo molecule intracellularly.

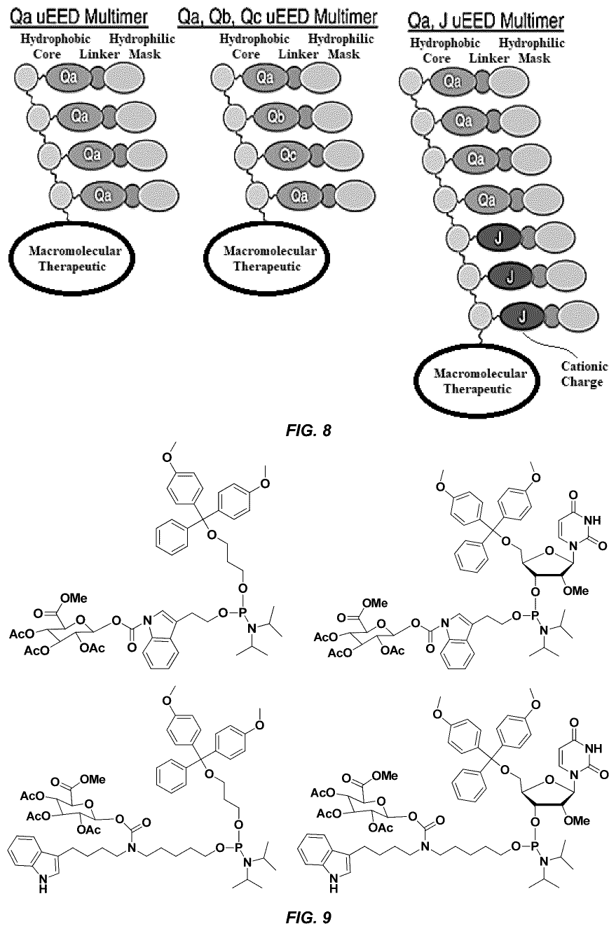

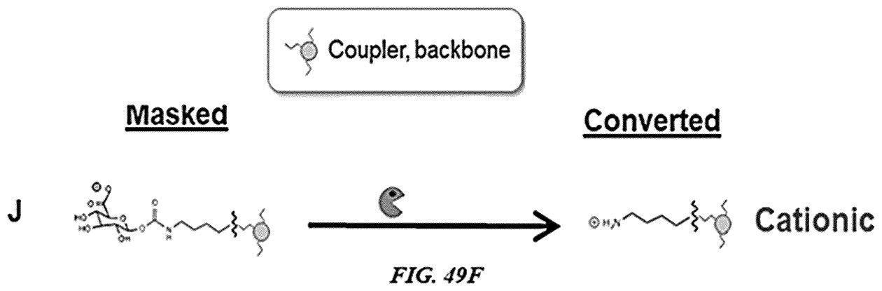

FIGS. 7 and 49 provide exemplary monomeric compounds of the disclosure. As will be noted, the monomeric compounds include similar modular domains. However, the domains are arranged in different orders. As depicted in FIG. 7, each “monomeric compound” comprises a hydrophilic mask domain, a hydrophobic or cationic domain and one or more linkers, wherein there is at least one linker that can be cleaved by endosomal agents such as an endosomal enzyme. An agent to be delivered (i.e., a cargo molecule) is linked to a plurality of monomeric units (“multimeric compounds”). A targeting moiety can be linked to the cargo moiety or the hydrophilic domain to promote endocytosis of the complex.

The following formula are useful in depicting the modular design of the uEEDs of the disclosure and represent certain non-limiting embodiments of the disclosure. In one embodiment, a monomeric compound has the structure of Formula I, II, III, IV, V or VI:

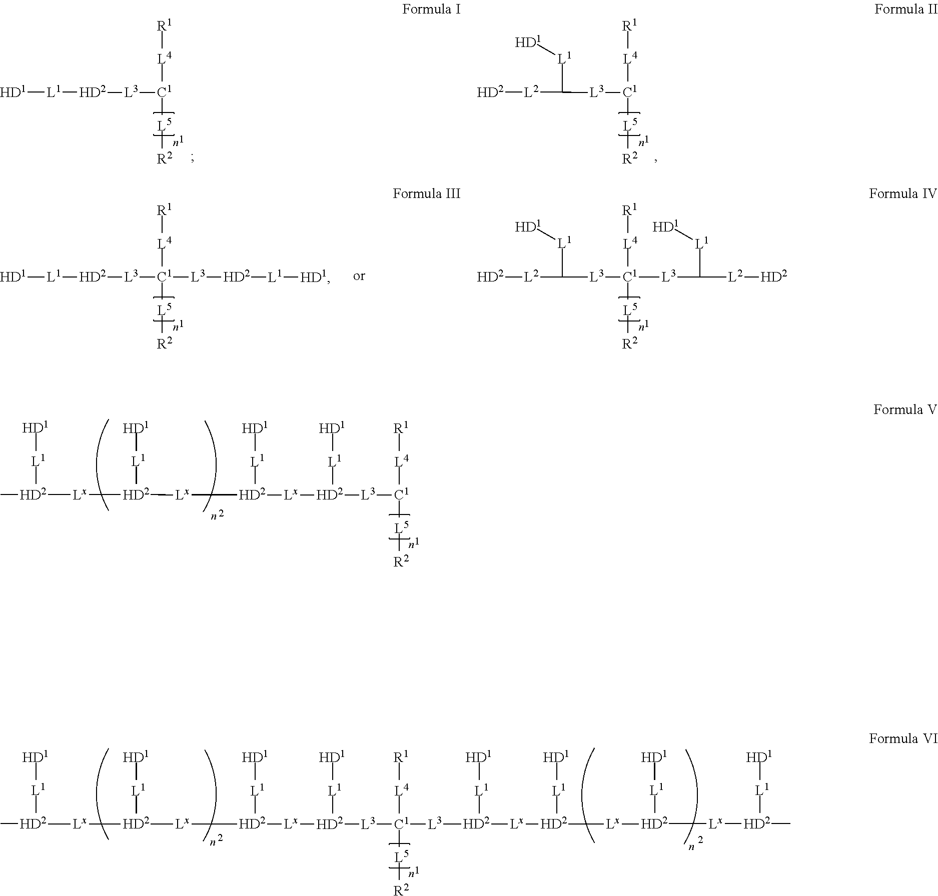

or a pharmaceutically acceptable salt, or solvate thereof, wherein, C¹ is the coupler domain; HD¹ is the hydrophilic mask domain; HD² is the hydrophobic domain or a cationic charge domain; L¹ is the biodegradable linker; L² is the second linker; L³ is the first linker; L⁴ is the third linker; L⁵ is the fourth linker (wherein the L4 and L5 linkers can have different numbers of carbons or other atoms); R¹ and R² are protecting of functional groups for solid-state synthesis; and n¹ is an integer selected from 0 or 1; n² is an integer selected from 0 to 10 or more (e.g., 1, 2, 3, 4, 5, 6, 7, 8, 9, 10, 11 etc.). In a further embodiment, the coupler domain comprises a phosphotriester group, or phosphoramidite group. In yet a further embodiment, the hydrophilic mask domain comprises a glycoside moiety. In another embodiment, the hydrophobic domain or the cationic charge domain is any functional group (or a plurality of such functional groups) which contains a primary, secondary or tertiary amino group, a lipid or a monomeric unit derived therefrom; a tocopherol; a hydrophobic oligomer or a monomeric unit derived therefrom; a hydrophobic polymer or a monomeric unit derived therefrom. In yet another embodiment, the hydrophobic domain comprises a lipid selected from a C8, C10, C12, C14, C16, or C18 lipid or derivative thereof. In a further embodiment, the hydrophobic domain comprises a monomeric unit derived from a lipid selected from fatty acid, fatty alcohol and any other lipidic molecule with at least two carbon units. In yet a further embodiment, the hydrophobic domain comprises a hydrophobic polymer selected from polymethylacryl, polyethylene, polystyrene, polyisobutane, polyester, polypeptide, or a derivative thereof. In another embodiment, the hydrophobic domain comprises one or more monomeric units derived from a hydrophobic polymer selected from the group consisting of: polyester, polyether, polycarbonate, polyanhydride, polyamide, polyacrylate, polymethacrylate, polyacrylamide, polysulfone, polyalkane, polyalkene, polyalkyne, polyanhydride, polyorthoester, N-isopropylacrylamide, methyl acrylate, ethyl acrylate, propyl acrylate, butyl acrylate, methyl methacrylate, ethyl methacrylate, propyl methacrylate, butyl methacrylate, acrylic acid, methacrylic acid, quaternary ammonium-modified acrylate, quaternary ammonium modified-methacrylate, acrylamide, caprolactone, lactide, and valerolactone. In yet another embodiment, the hydrophobic domain or the cationic charge domain comprises a 1H-indole group. In another embodiment, the biodegradable linker comprises a thioether group, a carbamate group, an ester group, a carbonate group, a urea group, or an enzyme cleavable peptidic linkage. In yet another embodiment, the biodegradable linker is an endosomal cleavable linker. In a certain embodiment, the endosomal cleavable linker comprises a carbamate group or a hydrazone group. In a further embodiment, the first linker comprises a group selected from an optionally substituted (C₁-C₆)alkyl, an optionally substituted (C₂-C₆)alkenyl, an optionally substituted (C₂-C₆)alkynyl, or an optionally substituted (C₁-C₆) alkoxy group. In another embodiment, the first linker comprises a group selected from ethyl, propyl, PEG₂, PEG₃ and PEG₄. In a further embodiment, the second linker is selected from an optionally substituted (C₁-C₆)alkyl, an optionally substituted (C₂-C₆)alkenyl, an optionally substituted (C₂-C₆)alkynyl, or an optionally substituted (C₁-C₆) alkoxy group. In yet a further embodiment, the second linker comprises a group selected from ethyl, propyl, PEG₂, PEG₃ and PEG₄. In another embodiment, the third and fourth linkers are selected from an optionally substituted (C₁-C₆)alkyl, an optionally substituted (C₂-C₆)alkenyl, an optionally substituted (C₂-C₆)alkynyl, an optionally substituted (C₁-C₆) alkoxy group, a uridine group, and a pyrimidine group. In a certain embodiment, the third and fourth linkers are a (C₁-C₆) alkyl or a uridine group.

In certain embodiments, the uEEDs were designed based on standard oligonucleotide solid-state synthesis parameters that include C1=phosphotriester with a P═O or P═S backbone, R1=DiMethoxylTrityl (DMT) protecting and leaving group, and R2=Phosphoramidite. Because the backbone required for solid-state synthesis is not directly a part of the active component of the uEED, any solid-state synthesis parameters could be included, but are not depicted, such as peptide synthesis, PMO synthesis, PNA synthesis, etc.

As generally depicted above in Formula I to VI, each domain of the uEED monomer can comprise various agents. For example, the Hydrophilic Mask domain can comprise one or more of the 40+ types of glycosides that are specifically cleaved by endosomal restricted glycosidases. This design avoids premature uEED activation outside of endosomes. In another embodiment, an Endosomal Cleavable Linker can be a self-immolating carbamate released as CO₂ or hydrozone or other endosome specific cleavable linker design. In still another embodiment, a Hydrophobic Core Endosomal Escape Domain (EED) can comprise single or multi-ring aromatic motifs, lipids or alkyl molecules or CPP domains. Where a Cationic domain is used, the cationic domain can be any nitrogen (N) containing primary secondary or tertiary amino group. In one embodiment, after endosomal cleavage and activation, the EED does not contain any residual hydrophilic motifs (charges, hydroxyls, etc.).

In a particular embodiment, the disclosure provides a monomeric compound comprising: a coupler domain; a hydrophobic domain or a cationic charge domain; a hydrophilic domain; an endosomally cleavable or degradable linker having a first end and a second end, wherein the endosomal cleavable or degradable linker is linked to the hydrophilic mask domain on the first end, and linked to the hydrophobic domain or cationic charge domain on the second end, or linked to a first linker on the second end; a first linker having a first end and a second end, wherein the first linker is linked to the coupler domain on the first end, and linked on the second end to the hydrophobic domain or cationic charge domain, or linked to a second linker; optionally, a second linker having a first end and a second end, wherein the second linker is linked to the hydrophobic domain or a cationic charge domain on the first end, and linked to the first linker on the second end; optionally, third and/or fourth linkers having a first end and a second end, wherein the first end is attached to the coupler domain, wherein the second end is attached to a functional group for solid-state synthesis.

In regards to the endosomal cleavable or degradable linker, the linker is susceptible to action of enzymes, or environments found in a subject's body. Such enzymes include, but are not limited to, esterases, glucosidases, and peptidases. Environments found in the subject body can be reducing environments found in lysosomes. Examples of degradable linkers include, but are not limited to, a thioether group, a carbamate group, an ester group, a carbonate group, a urea group, or an enzyme cleavable peptidic linkage. In a particular embodiment, the biodegradable linker is an endosomal cleavable linker. Endosomal cleavable linker can be a self-immolating carbamate released as CO₂ or hydrozone or another endosome specific linker design.

In regards to the coupler domain of the compounds disclosed herein, the coupler domain is used to form multimers from monomeric units using solid-state synthesis strategies. Examples of coupler domains include, but are not limited to, phosphotriester groups and phosphoramidite groups. Chemical synthesis molecules using solid state chemistry techniques can be accomplished using methods well known in the art, such as those set forth by Engels, et al., Angew. Chem. Intl. Ed., 28:716-734 (1989). These methods include, inter alia, the phosphotriester, phosphoramidite and H-phosphonate methods of polymer synthesis. Polymers comprising more than 10 monomeric compounds can be synthesized as several fragments, each fragment being up to about 10 monomers in length. In a particular embodiment, polymer-supported synthesis using standard phosphoramidite chemistry can be used to make the compounds of the disclosure.

In regards to the hydrophilic mask domain of the compounds disclosed herein, this domain comprises a positive charge moiety, or becomes positively charged when the moiety is exposed to certain pH environments, e.g., physiological pH, or acidic environments. Examples of moieties that can be used for hydrophilic mask domains, include, but are not limited to, glycosides including, but not limited to, b-Glucuronic Acid, a/b Galactose, N-Acetyl Glucosamine, Sialic Acid, Xylose, N-Acetyl Galactosamine, Mannose, Glucose and other glycosides. The purpose of said moieties/domains is to mask the hydrophobic domain or cationic charge domain, increase solubility of the compounds in aqueous environments. As there are more than 40 types of glycosides that are specifically cleaved by endosomal restricted glycosidases, the use of glycoside for the hydrophilic mask domain provides for additional functionality.

In regards to the hydrophobic or cationic domain of the compounds disclosed herein, the hydrophobic domain is typically composed from single or multi-ring aromatic motifs, lipids or alkyl molecules while the cationic domain includes one, typically a plurality of primary-secondary tertiary amino groups. All endosomes are composed of a lipid bilayer barrier that prevents >99% of macromolecular therapeutic from escaping the endosome into the cytoplasm and nucleus of cells. Thus, once the hydrophobic domain or cationic domain is ‘unmasked’ by the removal of the hydrophilic mask domain inside the endosome, the hydrophobic domain or cationic domains will interact with or integrate into the endosomal membrane, thereby disrupting membrane integrity and promoting endosomal escape of linked cargo. In a particular embodiment, the hydrophobic and/or cationic domain comprises moieties, including, but not limited to, functional groups that comprise primary, secondary or tertiary amino group, a lipid or a monomeric unit derived therefrom, a tocopherol, a hydrophobic oligomer or a monomeric unit derived therefrom, and a hydrophobic polymer or a monomeric unit derived therefrom. Other molecules including aromatic compounds such as indole and nitrogen containing aromatic multi-cyclic ring compounds are also contemplated. Examples of monomeric units derived from a lipid selected from fatty acid, fatty alcohol and any other lipidic molecule with at least two carbon units. Examples of hydrophobic polymer include, but are not limited to, polymethylacryl, polyethylene, polystyrene, polyisobutane, polyester, polypeptide, or a derivative thereof polyester, polyether, polycarbonate, polyanhydride, polyamide, polyacrylate, polymethacrylate, polyacrylamide, polysulfone, polyalkane, polyalkene, polyalkyne, polyanhydride, polyorthoester, N-isopropylacrylamide, methyl acrylate, ethyl acrylate, propyl acrylate, butyl acrylate, methyl methacrylate, ethyl methacrylate, propyl methacrylate, butyl methacrylate, acrylic acid, methacrylic acid, quaternary ammonium-modified acrylate, quaternary ammonium modified-methacrylate, acrylamide, caprolactone, lactide, and valerolactone.

The uEED constructs of the disclosure provide for the delivery of cargo molecules that are operably linked to a monomer or polymer of uEED constructs. The term “operably linked” or “operably associated” refers to functional linkage between two domains (e.g., a uEED and cargo domain).

The cargo domain can comprise a therapeutic agent and/or a diagnostic agent. Examples of therapeutic agents include, for example, thrombolytic agents and anticellular agents that kill or suppress the growth or cell division of disease-associated cells (e.g., cells comprising a cell proliferative disorder such as a neoplasm or cancer). Examples of effective thrombolytic agents are streptokinase and urokinase.

Exemplary therapeutic agents include, but are not limited to, antibiotics, antiproliferative agents, rapamycin macrolides, analgesics, anesthetics, antiangiogenic agents, vasoactive agents, anticoagulants, immunomodulators, cytotoxic agents, antiviral agents, antithrombotic drugs, antibodies, neurotransmitters, psychoactive drugs, and combinations thereof. Additional examples of therapeutic agents include, but are not limited to, cell cycle control agents; agents which inhibit cyclin protein production; cytokines, including, but not limited to, Interleukins 1 through 13 and tumor necrosis factors; anticoagulants, anti-platelet agents; TNF receptor domains and the like. Typically the therapeutic agent is neutral or positively charged. In certain instances, where the therapeutic agent is negatively charged, an additional charge neutralization moiety (e.g., a cationic peptide) can be used.

Effective anticellular agents include classical chemotherapeutic agents, such as steroids, antimetabolites, anthracycline, vinca alkaloids, antibiotics, alkylating agents, epipodophyllotoxin and anti-tumor agents such as neocarzinostatin (NCS), adriamycin and dideoxycytidine; mammalian cell cytotoxins, such as interferon-α (IFN-α), interferon-βγ (IFN-βγ), interleukin-12 (IL-12) and tumor necrosis factor-α (TNF-α); plant-, fungus- and bacteria-derived toxins, such as ribosome inactivating protein, gelonin, α-sarcin, aspergillin, restrictocin, ribonucleases, diphtheria toxin, Pseudomonas exotoxin, bacterial endotoxins, the lipid A moiety of a bacterial endotoxin, ricin A chain, deglycosylated ricin A chain and recombinant ricin A chain; as well as radioisotopes.

As used herein, a cargo domain can be (1) any heterologous polypeptide, or fragment thereof, (2) any polynucleotide (e.g., a ribozyme, RNAi (siRNA, shRNA etc), antisense molecule, polynucleotide, oligonucleotide and the like); (3) any small molecule, or (4) any diagnostic or therapeutic agent, that is capable of being linked or fused to a uEED. For example, the cargo domain can comprise any one or more of siRNA/siRNN RNAi triggers, ASOs, oligonucleotides (e.g., guide RNA (gRNA) or sequence encoding gRNA), CRISPR DNA/RNA editing, mRNA, DNA Vectors, Lipid Nanoparticles, proteins, peptides, large synthetic molecules. Any such cargo domain can be used to treat diseases and disorders recognized in the art including, but not limited to, cancer, inflammation, infection, autoimmune diseases, pain disorders, growth disorders, antiproliferative disorders, stem cell therapies, genetic abnormalities and the like.

The term “therapeutic” is used in a generic sense and includes treating agents, prophylactic agents, and replacement agents. Examples of therapeutic molecules include, but are not limited to, cell cycle control agents; agents which inhibit cyclin proteins, such as antisense polynucleotides to the cyclin G1 and cyclin D1 genes; growth factors such as, for example, epidermal growth factor (EGF), vascular endothelial growth factor (VEGF), erythropoietin, G-CSF, GM-CSF, TGF-α, TGF-β, and fibroblast growth factor; cytokines, including, but not limited to, Interleukins 1 through 13 and tumor necrosis factors; anticoagulants, anti-platelet agents; anti-inflammatory agents; tumor suppressor proteins; clotting factors including Factor VIII and Factor IX, protein S, protein C, antithrombin III, von Willebrand Factor, cystic fibrosis transmembrane conductance regulator (CFTR), and negative selective markers such as Herpes Simplex Virus thymidine kinase.

In addition, a cargo domain/molecule fused to a uEED can be a negative selective marker or “suicide” protein, such as, for example, the Herpes Simplex Virus thymidine kinase (TK) or cytosine deaminase (CD). Such a uEED linked to a suicide protein may be administered to a subject whereby tumor cells are selectively transduced. After the tumor cells are transduced with the kinase, an interaction agent, such as gancyclovir or acyclovir or 5-fluorocytosine (5-FC), is administered to the subject, whereby the transduced tumor cells are killed.

In addition, a cargo molecule can be a diagnostic agent such as an imaging agent. Exemplary diagnostic agents include, but are not limited to, imaging agents, such as those that are used in positron emission tomography (PET), computer assisted tomography (CAT), single photon emission computerized tomography, X-ray, fluoroscopy, and magnetic resonance imaging (MRI). Suitable materials for use as contrast agents in MRI include, but are not limited to, gadolinium chelates, as well as iron, magnesium, manganese, copper, and chromium chelates. Examples of materials useful for CAT and X-rays include, but are not limited to, iodine based materials.

Examples of radioimaging agents emitting radiation (detectable radio-labels) that may be suitable are exemplified by indium-111, technetium-99, or low dose iodine-131. Detectable labels, or markers, for use in conjunction with or attached to the nucleic acid constructs of the disclosure as auxiliary moieties may be a radiolabel, a fluorescent label, a nuclear magnetic resonance active label, a luminescent label, a chromophore label, a positron emitting isotope for PET scanner, a chemiluminescence label, or an enzymatic label. Fluorescent labels include, but are not limited to, green fluorescent protein (GFP), fluorescein, and rhodamine. The label may be for example a medical isotope, such as for example and without limitation, technetium-99, iodine-123 and -131, thallium-201, gallium-67, fluorine-18, indium-111, etc.

Thus, it is to be understood that the disclosure is not to be limited to any particular cargo domain used for diagnosis and/or treatment of any particular disease or disorder. Rather, the cargo domain can be any molecule or agent known or used in the art for treatment or diagnostics of a disease or disorder.

When the cargo domain is a polypeptides, the polypeptide can comprise L-optical isomer or the D-optical isomer of amino acids or a combination of both. Polypeptides that can be used in the disclosure include modified sequences such as glycoproteins, retro-inverso polypeptides, D-amino acid modified polypeptides, and the like. A polypeptide includes naturally occurring proteins, as well as those which are recombinantly or synthetically synthesized. “Fragments” are a portion of a polypeptide. The term “fragment” refers to a portion of a polypeptide which exhibits at least one useful epitope or functional domain. The term “functional fragment” refers to fragments of a polypeptide that retain an activity of the polypeptide. Functional fragments can vary in size from a polypeptide fragment as small as an epitope capable of binding an antibody molecule, to a large polypeptide capable of participating in the characteristic induction or programming of phenotypic changes within a cell. An “epitope” is a region of a polypeptide capable of binding an immunoglobulin generated in response to contact with an antigen. Small epitopes of receptor ligands can be useful in the methods of the invention so long as it retains the ability to interact with the receptor.

In some embodiments, retro-inverso peptides are used. “Retro-inverso” means an amino-carboxy inversion as well as enantiomeric change in one or more amino acids (i.e., levantory (L) to dextrorotary (D)). A polypeptide encompasses, for example, amino-carboxy inversions of the amino acid sequence, amino-carboxy inversions containing one or more D-amino acids, and non-inverted sequence containing one or more D-amino acids. Retro-inverso peptidomimetics that are stable and retain bioactivity can be devised as described by Brugidou et al. (Biochem. Biophys. Res. Comm. 214(2): 685-693, 1995) and Chorev et al. (Trends Biotechnol. 13(10): 438-445, 1995).

uEED monomers are designed with chemical coupling agents that allow for synthesis of uEED multimers on solid-state synthesizers. The methods of the disclosure allow for controlling the optimal number of uEED monomer units as well as the ability to incorporate either a single type of uEED monomer or a variety of different types of uEED monomers to generate structurally well-defined diverse uEED multimer libraries capable of optimizing endosomal escape and delivery of a wide variety of a given type of macromolecular cargo. For instance, delivery of a siRNA (˜14 kDa) may require a single uEED hexamer, whereas delivery of a LNP (˜100 megaDa) may require many uEED decamers on its surface. Regardless of the number or type of monomer units, all uEED multimers are based on the same bio-mimic design principle. uEEDs also contain a motif for conjugation to all classes of macromolecular therapeutics at any desired number.

Because any given macromolecular therapeutic class may have different optimal requirements for endosomal escape, the uEED design is built on the synthesis of a uEED monomer that is capable of undergoing (and surviving) solid-state synthesis. This approach allows for the synthesis of a collection of uEED multimers that contain any number of uEED monomer units from 2, 3, 4, 5, 6 . . . to 20 or more. Each uEED multimer contains a conjugation handle (Click, HyNic, Aminooxy, etc.) for conjugation to macromolecular therapeutics (e.g., cargo). After solid-state synthesis, uEEDs are deprotected to remove all protecting groups and purified by HPLC.

In certain embodiments, a uEED can be linked to a cargo domain and may further include a targeting moiety. The disclosure provides for one or more targeting moieties which can be attached to a uEED construct disclosed herein as an auxiliary moiety, for example as a targeting auxiliary moiety. A targeting moiety is selected based on its ability to target constructs of the disclosure to a desired or selected cell population that expresses the corresponding binding partner (e.g., either the corresponding receptor or ligand) for the selected targeting moiety. A targeting moiety is also selected based upon its ability to either induce endocytosis or attach to a cell surface protein that endocytosis. For example, a construct of the disclosure could be targeted to cells expressing epidermal growth factor receptor (EGFR) by selected epidermal growth factor (EGF) as the targeting moiety that induces endocytosis.

In one embodiment, the targeting moiety is a receptor binding domain. In another embodiment, the targeting moiety is or specifically binds to a protein selected from the group comprising insulin, insulin-like growth factor receptor 1 (IGF1R), IGF2R, insulin-like growth factor (IGF; e.g., IGF 1 or 2), mesenchymal epithelial transition factor receptor (c-met; also known as hepatocyte growth factor receptor (HGFR)), hepatocyte growth factor (HGF), epidermal growth factor receptor (EGFR), epidermal growth factor (EGF), heregulin, fibroblast growth factor receptor (FGFR), platelet-derived growth factor receptor (PDGFR), platelet-derived growth factor (PDGF), vascular endothelial growth factor receptor (VEGFR), vascular endothelial growth factor (VEGF), tumor necrosis factor receptor (TNFR), tumor necrosis factor alpha (TNF-α), TNF-β, folate receptor (FOLR), folate, transferring, transferrin receptor (TfR), mesothelin, Fc receptor, c-kit receptor, c-kit, an integrin (e.g., an α4 integrin or a β-1 integrin), P-selectin, sphingosine-1-phosphate receptor-1 (S1PR), hyaluronate receptor, leukocyte function antigen-1 (LFA-1), CD4, CD11, CD18, CD20, CD25, CD27, CD52, CD70, CD80, CD85, CD95 (Fas receptor), CD106 (vascular cell adhesion molecule 1 (VCAM1), CD166 (activated leukocyte cell adhesion molecule (ALCAM)), CD178 (Fas ligand), CD253 (TNF-related apoptosis-inducing ligand (TRAIL)), ICOS ligand, CCR2, CXCR3, CCR5, CXCL12 (stromal cell-derived factor 1 (SDF-1)), interleukin 1 (IL-1), IL-1ra, IL-2, IL-3, IL-4, IL-6, IL-7, IL-8, CTLA-4, MART-1, gp100, MAGE-1, ephrin (Eph) receptor, mucosal addressin cell adhesion molecule 1 (MAdCAM-1), carcinoembryonic antigen (CEA), Lewis^{Y}, MUC-1, epithelial cell adhesion molecule (EpCAM), cancer antigen 125 (CA125), prostate specific membrane antigen (PSMA), TAG-72 antigen, and fragments thereof. In a further embodiment, the targeting moiety is erythroblastic leukemia viral oncogene homolog (ErbB) receptor (e.g., ErbB1 receptor; ErbB2 receptor; ErbB3 receptor; and ErbB4 receptor).

The targeting moiety can also be selected from bombesin, gastrin, gastrin-releasing peptide, tumor growth factors (TGF), such as TGF-α and TGF-β, and vaccinia virus growth factor (VVGF). Non-peptidyl ligands can also be used as the targeting moiety and may include, for example, steroids, carbohydrates, vitamins, and lectins. The targeting moiety may also be selected from a peptide or polypeptide, such as somatostatin (e.g., a somatostatin having the core sequence cyclo[Cys-Phe-D-Trp-Lys-Thr-Cys], and in which, for example, the C-terminus of the somatostatin analog is: Thr-NH₂), a somatostatin analog (e.g., octreotide and lanreotide), bombesin, a bombesin analog, or an antibody, such as a monoclonal antibody.

Other peptides or polypeptides for use as a targeting auxiliary moiety in uEED constructs of the disclosure can be selected from KISS peptides and analogs, urotensin II peptides and analogs, GnRH I and II peptides and analogs, depreotide, vapreotide, vasoactive intestinal peptide (VIP), cholecystokinin (CCK), RGD-containing peptides, melanocyte-stimulating hormone (MSH) peptide, neurotensin, calcitonin, peptides from complementarity determining regions of an antitumor antibody, glutathione, YIGSR (leukocyte-avid peptides, e.g., P483H, which contains the heparin-binding region of platelet factor-4 (PF-4) and a lysine-rich sequence), atrial natriuretic peptide (ANP), β-amyloid peptides, delta-opioid antagonists (such as ITIPP (psi)), annexin-V, endothelin, leukotriene B4 (LTB4), chemotactic peptides (e.g., N-formyl-methionyl-leucyl-phenylalanine-lysine (fMLFK)), GP IIb/IIIa receptor antagonists (e.g., DMP444), human neutrophil elastase inhibitor (EPI-HNE-2 and EPI-HNE-4), plasmin inhibitor, antimicrobial peptides, aphicide (P280 and P274), thrombospondin receptor (including analogs such as TP-1300), bitistatin, pituitary adenylyl cyclase type I receptor (PAC1), fibrin α-chain, peptides derived from phage display libraries, and conservative substitutions thereof.

Immunoreactive ligands for use as a targeting moiety in uEED constructs of the disclosure include an antigen-recognizing immunoglobulin (also referred to as “antibody”), or antigen-recognizing fragment thereof that is capable of inducing endocytosis. As used herein, “immunoglobulin” refers to any recognized class or subclass of immunoglobulins such as IgG, IgA, IgM, IgD, or IgE. Typical are those immunoglobulins which fall within the IgG class of immunoglobulins. The immunoglobulin can be derived from any species. Typically, however, the immunoglobulin is of human, murine, or rabbit origin. In addition, the immunoglobulin may be polyclonal or monoclonal, but is typically monoclonal.

Targeting moieties of the disclosure may include an antigen-recognizing immunoglobulin fragment. Such immunoglobulin fragments may include, for example, the Fab′, F(ab′)₂, Fᵥ or Fab fragments, single-domain antibody, ScFv, or other antigen-recognizing immunoglobulin fragments. Fc fragments may also be employed as targeting moieties. Such immunoglobulin fragments can be prepared, for example, by proteolytic enzyme digestion, for example, by pepsin or papain digestion, reductive alkylation, or recombinant techniques. The materials and methods for preparing such immunoglobulin fragments are well-known to those skilled in the art. See Parham, J. Immunology, 131, 2895, 1983; Lamoyi et al., J. Immunological Methods, 56, 235, 1983.

Targeting moieties of the disclosure include those targeting moieties which are known in the art but have not been provided as a particular example in this disclosure that either induce endocytosis or are endocytosed.

Peptide linkers that can be used in the constructs and methods of the disclosure will typically comprise up to about 20 or 30 amino acids, commonly up to about 10 or 15 amino acids, and still more often from about 1 to 5 amino acids. The linker sequence is generally flexible so as not to hold the fusion molecule in a single rigid conformation. The linker sequence can be used, e.g., to space one domain from another domain. For example, the peptide linker sequence can be positioned between the hydrophilic domain and the cationic domain.

The disclosure includes all isomeric (e.g., enantiomeric, diastereomeric, and geometric (or conformational)) forms of the compounds; for example, syn and anti isomers, the R and S configurations for each asymmetric center, Z and E double bond isomers, and Z and E conformational isomers. Therefore, single stereochemical isomers as well as enantiomeric, diastereomeric, and geometric (or conformational) mixtures of the compounds of the disclosure are contemplated herein. Unless otherwise stated, all tautomeric forms of the compounds of the disclosure are contemplated herein. The disclosure includes all pharmaceutically acceptable isotopically-labeled compounds of the disclosure, wherein one or more atoms are replaced by atoms having the same atomic number, but an atomic mass or mass number different from the atomic mass or mass number usually found in nature. Examples of isotopes suitable for inclusion in the compounds of the disclosure comprises isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S.

Salts derived from appropriate bases include alkali metal, alkaline earth metal, and ammonium salts. Representative alkali or alkaline earth metal salts include sodium, lithium, potassium, calcium, magnesium, and the like. One class of salts includes the pharmaceutically acceptable salts. The term “pharmaceutically acceptable salt,” as use herein, represents those salts which are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting the free base group with a suitable organic acid. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts and the like. Representative alkali or alkaline earth metal salts include sodium, lithium, potassium, calcium, magnesium and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine and the like.

A pharmaceutical composition according to the disclosure can be prepared to include a compound of the disclosure, into a form suitable for administration to a subject using carriers, excipients, and additives or auxiliaries. Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol, and polyhydric alcohols. Intravenous vehicles include fluid and nutrient replenishers. Preservatives include antimicrobial, anti-oxidants, chelating agents, and inert gases. Other pharmaceutically acceptable carriers include aqueous solutions, non-toxic excipients, including salts, preservatives, buffers and the like, as described, for instance, in Remington's Pharmaceutical Sciences, 15th ed., Easton: Mack Publishing Co., 1405-1412, 1461-1487 (1975), and The National Formulary XIV., 14th ed., Washington: American Pharmaceutical Association (1975), the contents of which are hereby incorporated by reference. The pH and exact concentration of the various components of the pharmaceutical composition are adjusted according to routine skills in the art. See Goodman and Gilman's, The Pharmacological Basis for Therapeutics (7th ed.).

The pharmaceutical compositions according to the disclosure may be administered locally or systemically. By “therapeutically effective dose” is meant the quantity of a fusion polypeptide according to the disclosure necessary to prevent, to cure, or at least partially arrest the symptoms of a disease or disorder (e.g., to inhibit cellular proliferation). Amounts effective for this use will, of course, depend on the severity of the disease and the weight and general state of the subject. Typically, dosages used in vitro may provide useful guidance in the amounts useful for in situ administration of the pharmaceutical composition, and animal models may be used to determine effective dosages for treatment of particular disorders. Various considerations are described, e.g., in Langer, Science, 249: 1527, (1990); Gilman et al. (eds.) (1990), each of which is herein incorporated by reference.

As used herein, “administering a therapeutically effective amount” is intended to include methods of giving or applying a pharmaceutical composition of the disclosure to a subject that allow the composition to perform its intended therapeutic function. The therapeutically effective amounts will vary according to factors, such as the degree of disease in a subject, the age, sex, and weight of the individual. Dosage regimen can be adjusted to provide the optimum therapeutic response. For example, several divided doses can be administered daily or the dose can be proportionally reduced as indicated by the exigencies of the therapeutic situation.

The pharmaceutical composition can be administered in a convenient manner, such as by injection (e.g., subcutaneous, intravenous, intracerebrally, intraspinal, intraocular, and the like), oral administration, inhalation, transdermal application, or rectal administration. Depending on the route of administration, the pharmaceutical composition can be coated with a material to protect the pharmaceutical composition from the action of enzymes, acids, and other natural conditions that may inactivate the pharmaceutical composition. The pharmaceutical composition can also be administered parenterally or intraperitoneally. Dispersions can also be prepared in glycerol, liquid polyethylene glycols, and mixtures thereof, and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersions. The composition will typically be sterile and fluid to the extent that easy syringability exists. Typically, the composition will be stable under the conditions of manufacture and storage and preserved against the contaminating action of microorganisms, such as bacteria and fungi. The carrier can be a solvent or dispersion medium containing, for example, water, ethanol, polyol (for example, glycerol, propylene glycol, and liquid polyethyelene glycol, and the like), suitable mixtures thereof, and vegetable oils. The proper fluidity can be maintained, for example, by the use of a coating, such as lecithin, by the maintenance of the required particle size, in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents, for example, sugars, polyalcohols, such as mannitol, sorbitol, or sodium chloride are used in the composition. Prolonged absorption of the injectable compositions can be brought about by including in the composition an agent that delays absorption, for example, aluminum monostearate and gelatin.

Sterile injectable solutions can be prepared by incorporating the pharmaceutical composition in the required amount in an appropriate solvent with one or a combination of ingredients enumerated above, as required, followed by filtered sterilization. Generally, dispersions are prepared by incorporating the pharmaceutical composition into a sterile vehicle that contains a basic dispersion medium and the required other ingredients from those enumerated above.

The pharmaceutical composition can be orally administered, for example, with an inert diluent or an assimilable edible carrier. The pharmaceutical composition and other ingredients can also be enclosed in a hard or soft-shell gelatin capsule, compressed into tablets, or incorporated directly into the subject's diet. For oral therapeutic administration, the pharmaceutical composition can be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations can, of course, be varied and can conveniently be between about 5% to about 80% of the weight of the unit.

The tablets, troches, pills, capsules, and the like can also contain the following: a binder, such as gum tragacanth, acacia, corn starch, or gelatin; excipients such as dicalcium phosphate; a disintegrating agent, such as corn starch, potato starch, alginic acid, and the like; a lubricant, such as magnesium stearate; and a sweetening agent, such as sucrose, lactose or saccharin, or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar, or both. A syrup or elixir can contain the agent, sucrose as a sweetening agent, methyl and propylparabens as preservatives, a dye, and flavoring, such as cherry or orange flavor. Of course, any material used in preparing any dosage unit form should be pharmaceutically pure and substantially non-toxic in the amounts employed. In addition, the pharmaceutical composition can be incorporated into sustained-release preparations and formulations.

Thus, a “pharmaceutically acceptable carrier” is intended to include solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the pharmaceutical composition, use thereof in the therapeutic compositions and methods of treatment is contemplated. Supplementary active compounds can also be incorporated into the compositions.

It is especially advantageous to formulate parenteral compositions in dosage unit form for ease of administration and uniformity of dosage. “Dosage unit form” as used herein, refers to physically discrete units suited as unitary dosages for the subject to be treated; each unit containing a predetermined quantity of pharmaceutical composition is calculated to produce the desired therapeutic effect in association with the required pharmaceutical carrier. The specification for the dosage unit forms of the disclosure are related to the characteristics of the pharmaceutical composition and the particular therapeutic effect to be achieve.

The principal pharmaceutical composition is compounded for convenient and effective administration in effective amounts with a suitable pharmaceutically acceptable carrier in an acceptable dosage unit. In the case of compositions containing supplementary active ingredients, the dosages are determined by reference to the usual dose and manner of administration of the said ingredients.

The working examples below are provided to illustrate, not limit, the invention. Various parameters of the scientific methods employed in these examples are described in detail below and provide guidance for practicing the invention in general.

### Examples

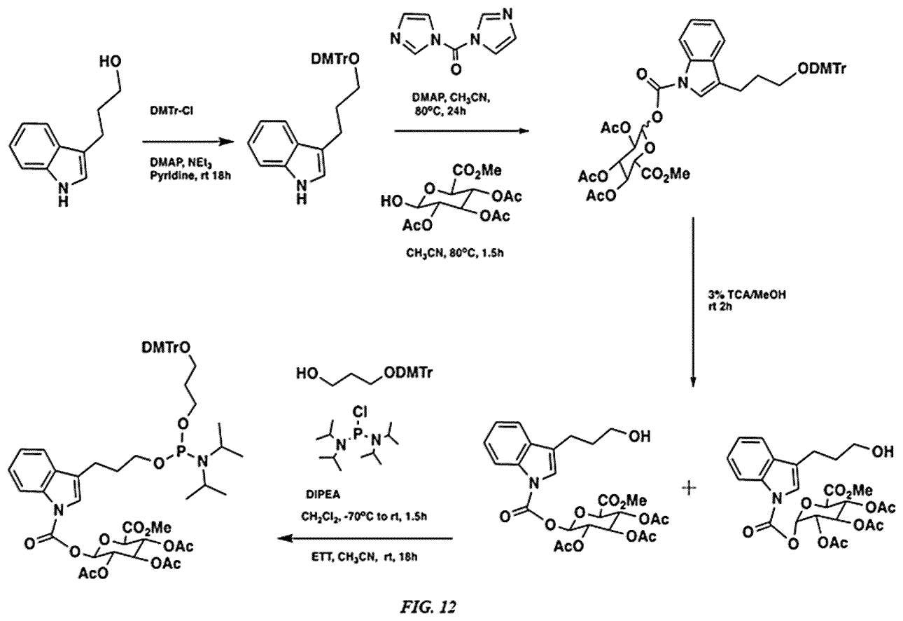

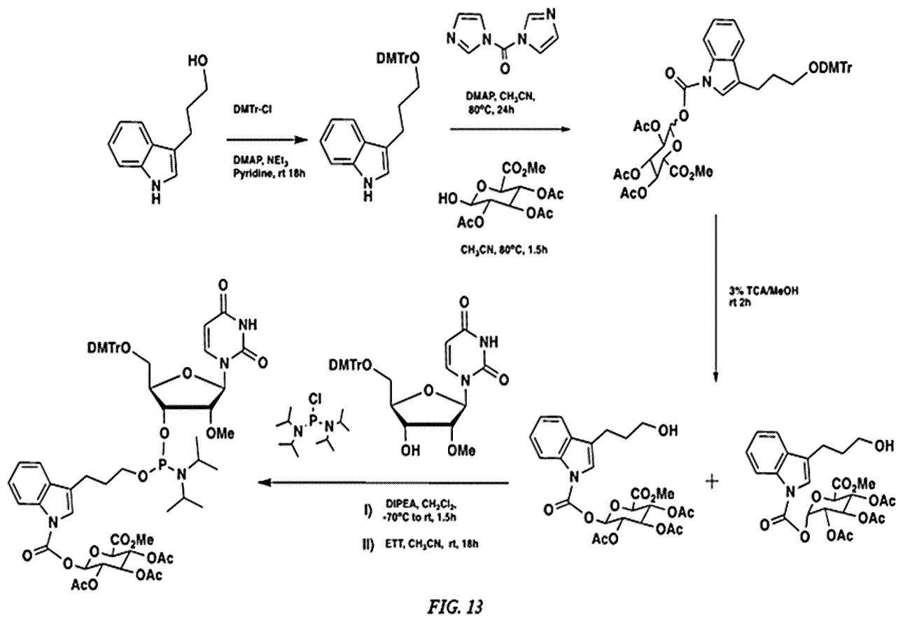

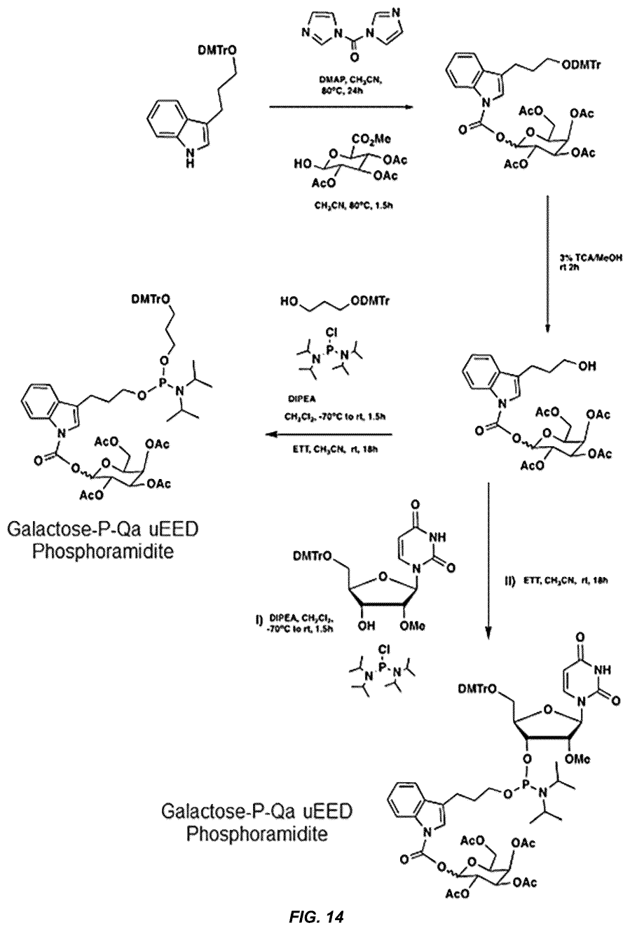

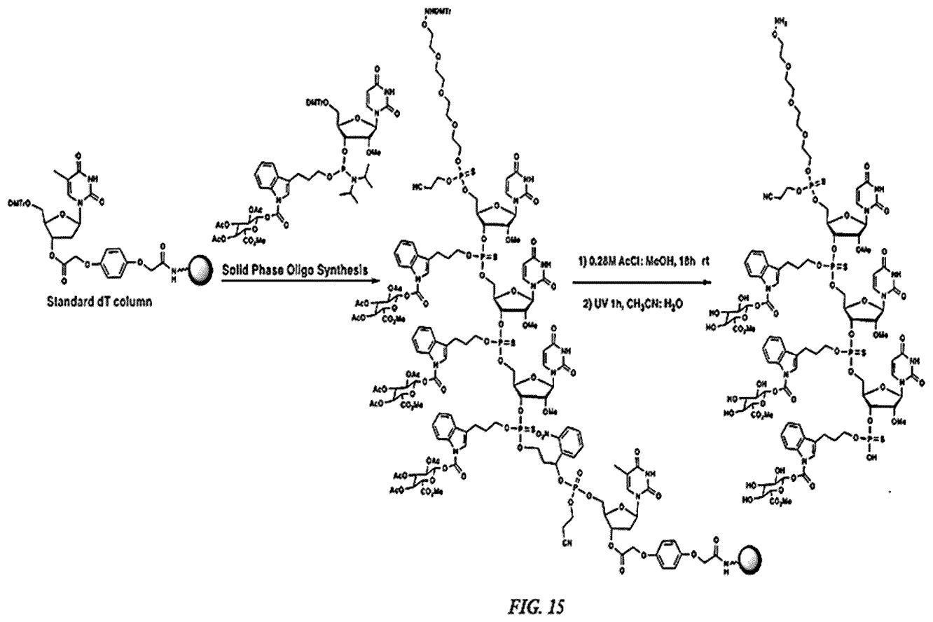

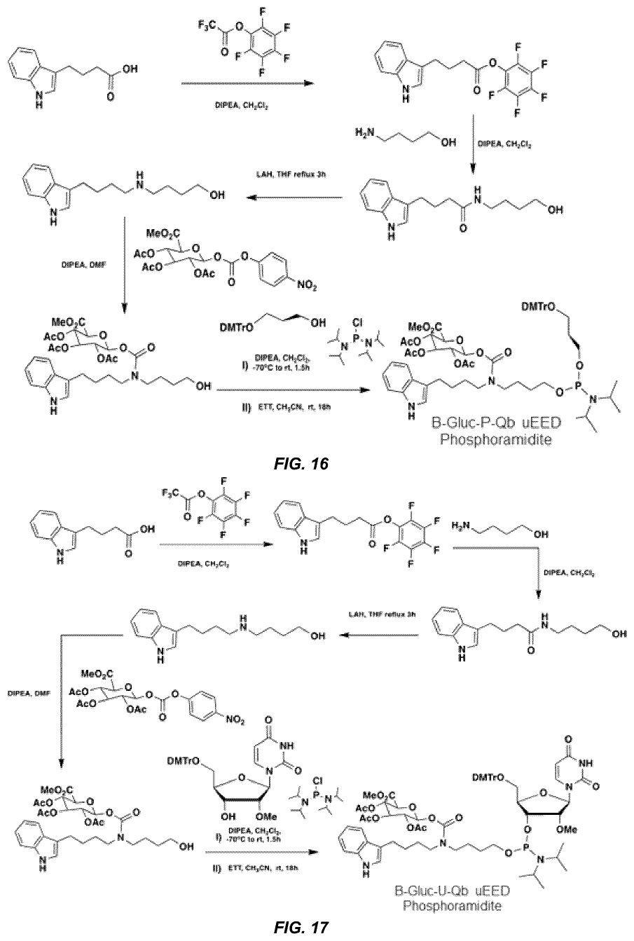

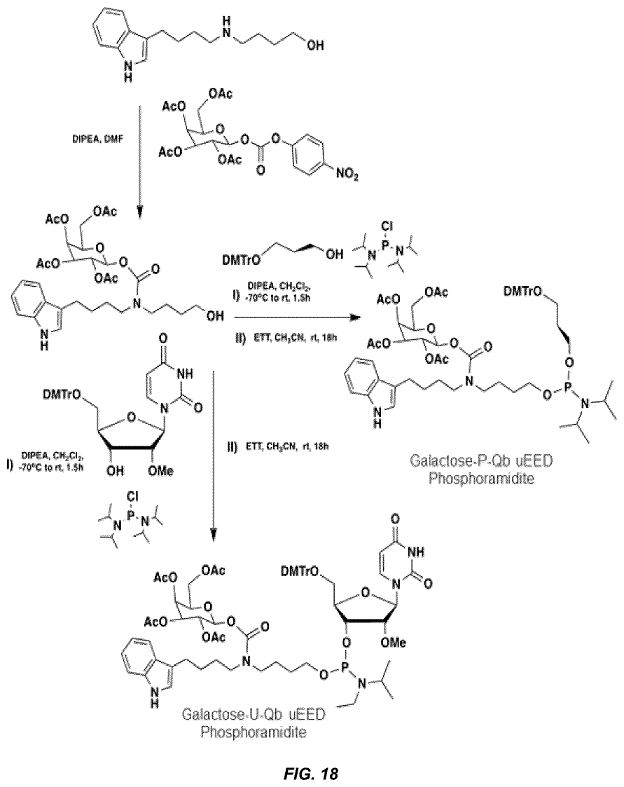

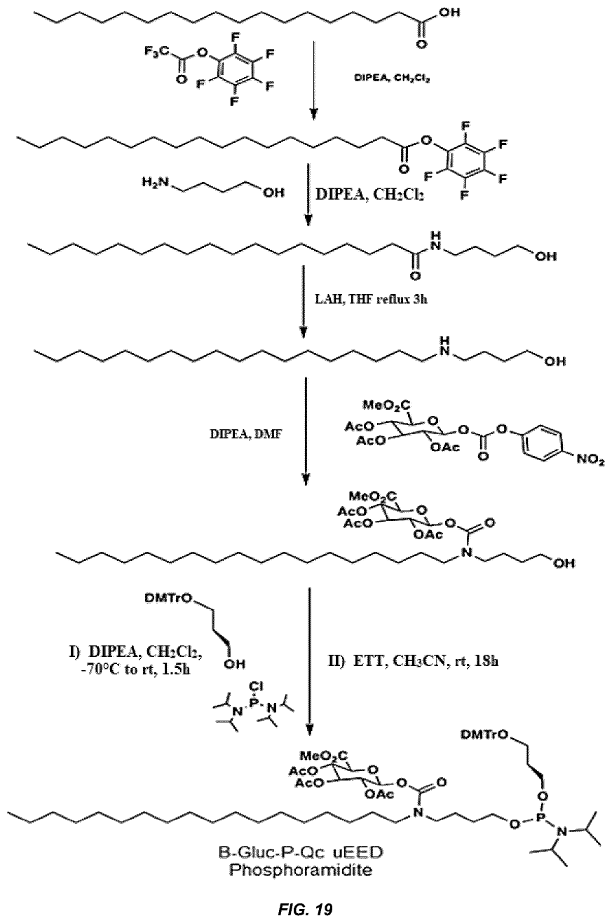

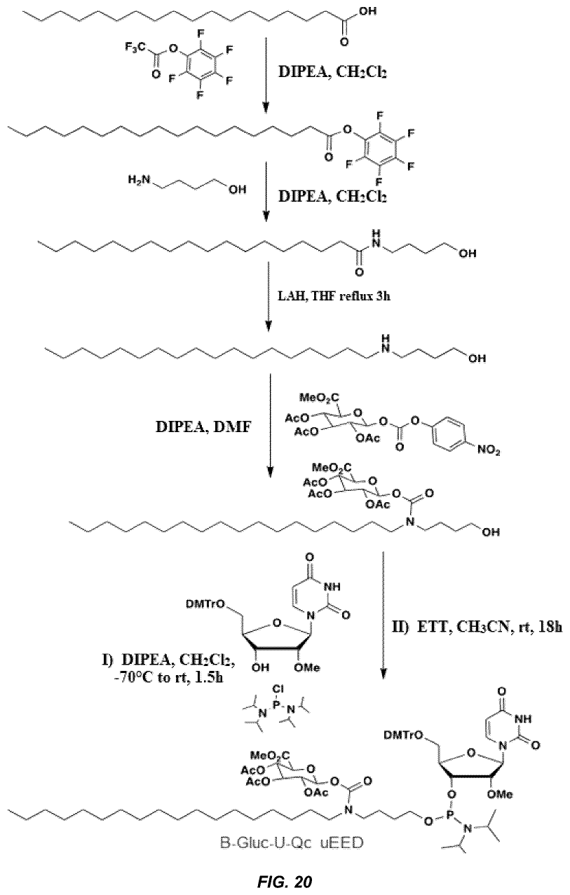

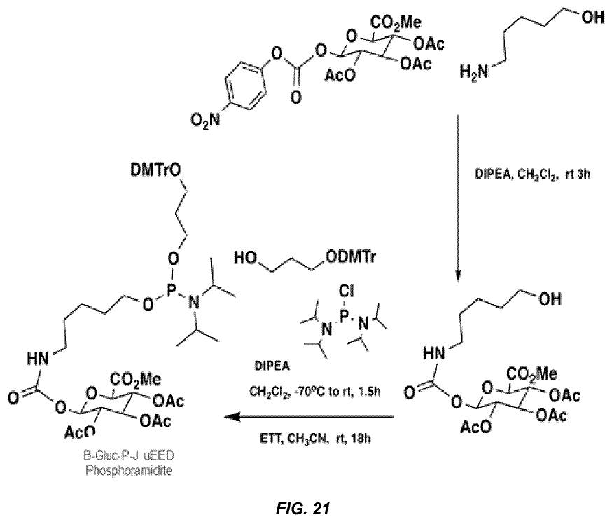

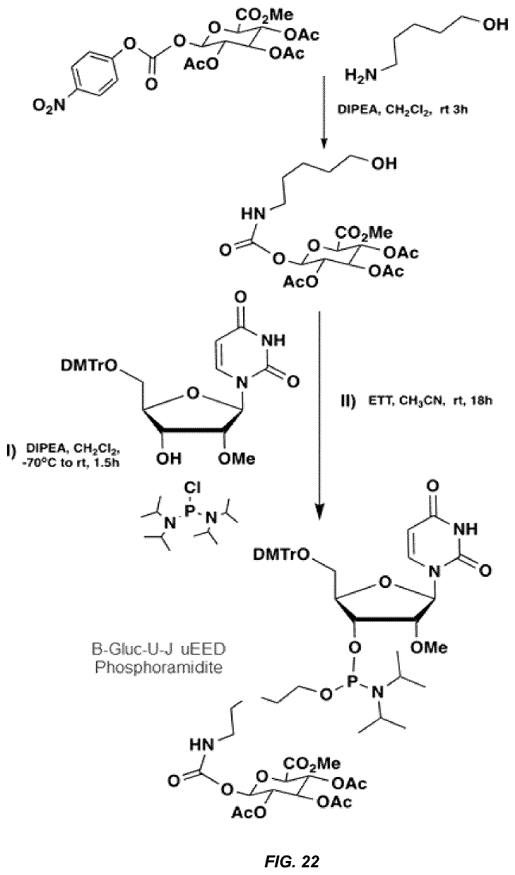

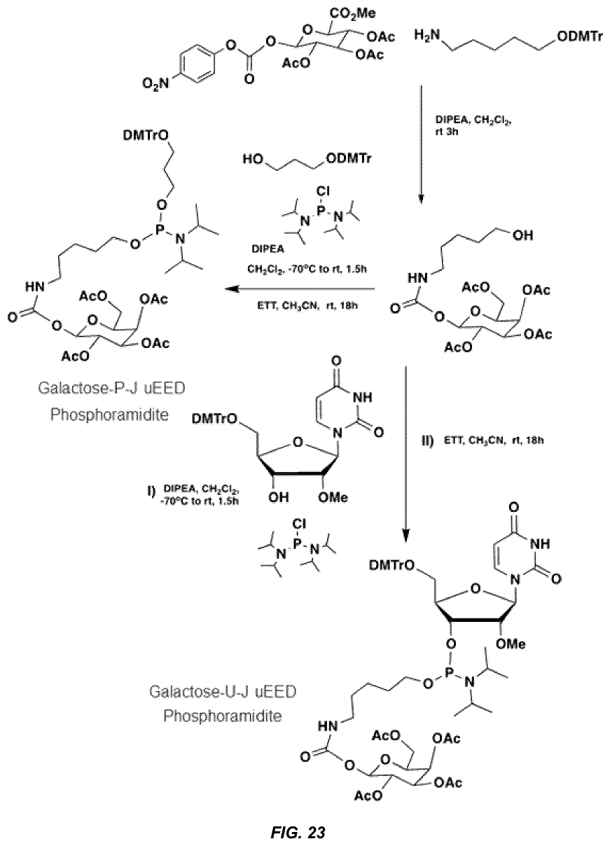

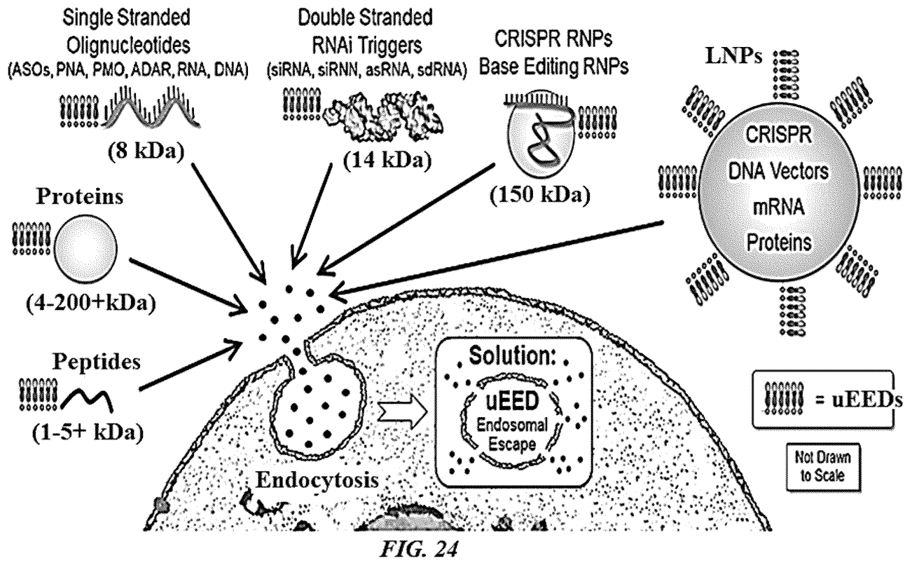

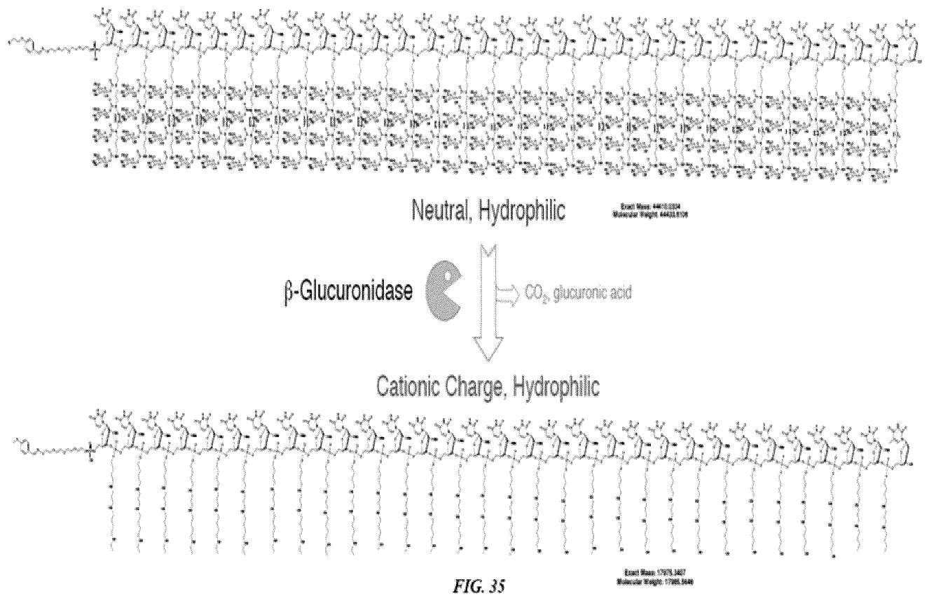

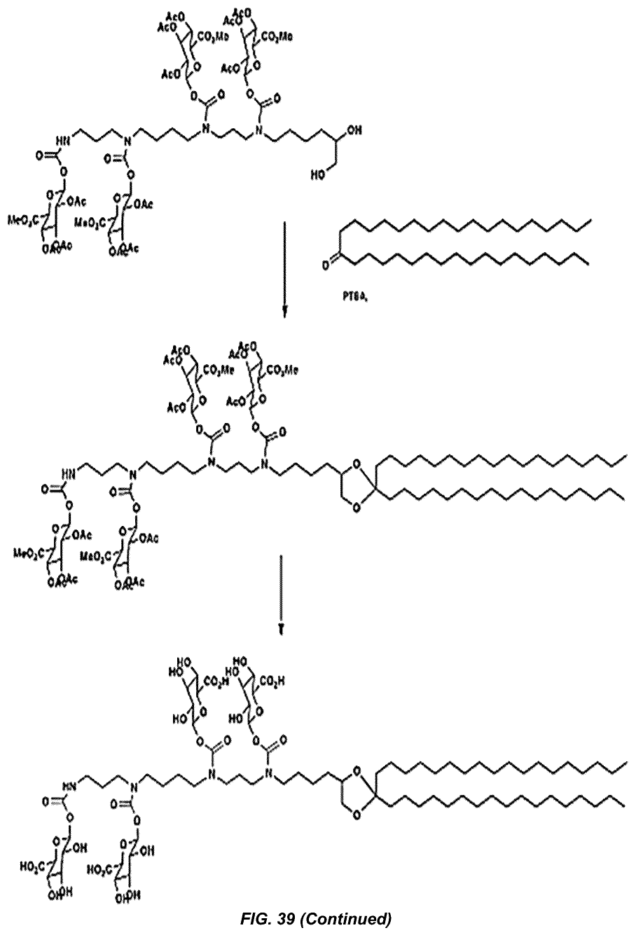

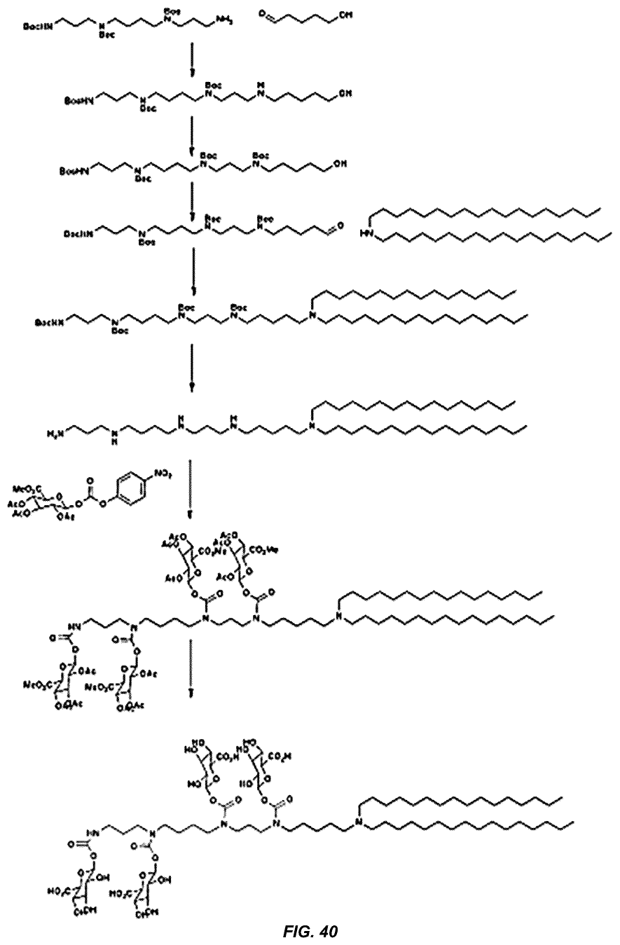

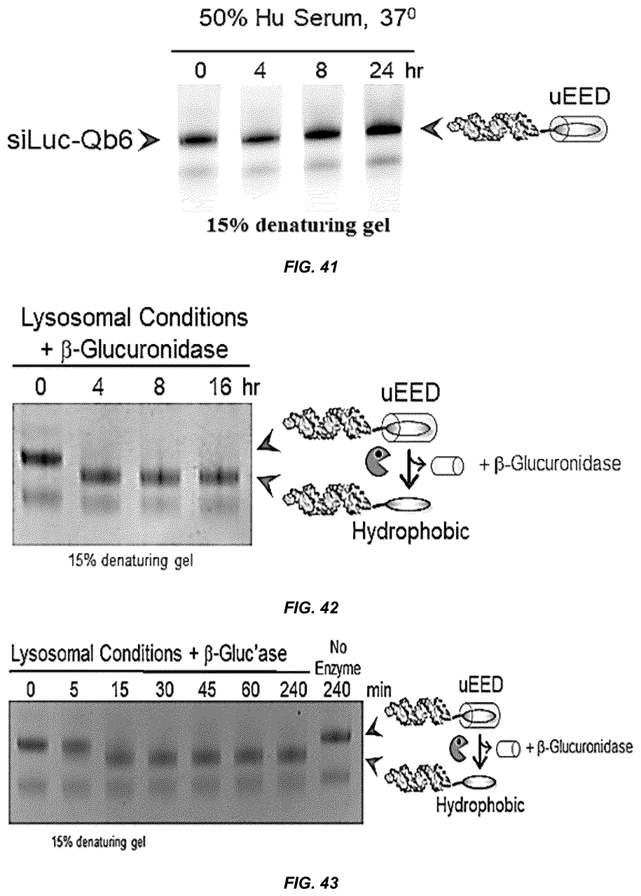

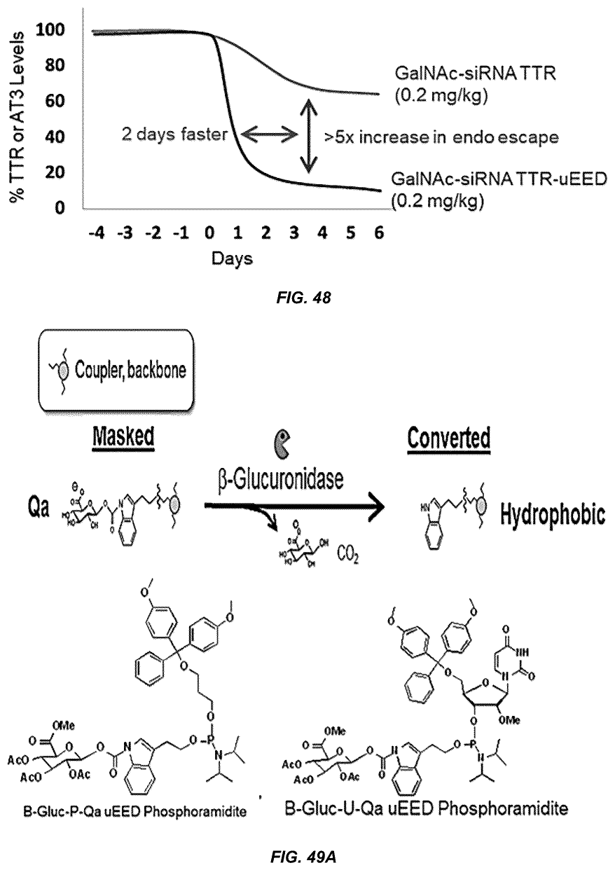

Synthesis of phosphoramidite uEED precursors. The phosphoramidite uEED precursors were synthesized using solid-state phosphoramidite techniques with the synthetic routes and reactions presented in FIGS. 12-23, 32, 38-40 and 45.

Processing of phosphoramidite uEED precursors into uEEDs. Removal of the β-Glucuronide methyl ester protecting group: 50 uL of phosphoramidite uEED precursors in DMF:H₂O (1:1) was placed in an Eppendorf tube, followed by addition of 100 uL mouse serum (containing high esterase activity) and the reaction mixture was incubated for 4 h at ambient temperature. The methyl ester group was calc. to be removed in <15 min. The reaction mixture was treated with 300 uL acetonitrile to precipitate plasma proteins. The sample was centrifuged for 10 min, and the supernatant dried by centrifugal evaporation to afford the uEED multimers.

B-Glucuronidase Assay. Unpurified synthesized uEED multimers were dissolved in 40 μL water. To which was added 504 of NaOAc (pH 6) and 10 μL of bovine derived Beta-Glucuronidase (500 U/mL in 0.2% NaCl). The samples were incubated at 37° C. overnight. The reaction mixture was treated with acetonitrile (300 uL), centrifuged for 10 min, and the supernatant was syringe filtered (0.2 micron) and analyzed by CombiFlash or ESI mass spectrometry.

Analysis of uEEDs by CombiFlash and electrospray ionization mass spectrometry (ESI-MS). The Beta-Glucuronidase treated samples after syringe filtration were injected (200 μL) into a C18 HPLC column. A solvent gradient of B=90% acetonitrile/water, and A of 50 mM TEAA/water was used. Fractions were collected and uEED hydrolysis was determined by ESI-MS analysis.

To test the metabolic stability of Qa uEED, Qa uEED was incubated with mouse serum, which contains significant and well-known metabolic enzymatic activities. After 4 hours of treatment with mouse serum, the Qa functional group of the Qa uEED was analyzed by C18 HPLC and ESI mass spectrometry. The glucuronic acid moiety of Qa uEED was highly metabolically stable in mouse serum and showed no signs of metabolic degradation.

Delivery of RNA Oligonucleotides into Mammalian Cells. To test the ability of uEEDs to enhance endosomal escape and hence delivery of RNA oligonucleotide therapeutics into cells in vitro, a GalNAc targeting domain containing a terminal azide was conjugated to a 5′ end BCN group on a Luciferase siRNA Passenger (sense) strand via Click conjugation chemistry. The GalNAc-siRNA conjugate is then conjugated to various uEEDs containing a terminal tetrazine group that will drive conjugation to a 3′ trans cyclooctene (TCO) group on the Passenger strand. Together, this single pot dual conjugation approach allows for site selective conjugation and the precise generation of GalNAc-siRNA-uEED constructs, where various uEED multimers containing from 2, 4, 6 . . . to 20 or more uEED monomers can be rapidly tested for increased endosomal escape and delivery into the cytoplasm by RNAi knockdown of the Luciferase reporter gene.

Primary murine hepatocytes from ROSA26-Lox-Stop-lox (LSL) Luciferase mice are pretreated a week (or more) prior with an i. v. administered Adenovirus-Cre to recombine out the LSL DNA segment and thereby constitutively express Luciferase, are isolated per standard protocols and placed into cell culture. Luciferase expressing hepatocytes plated in 24 well plates are treated with various GalNAc-Luc siRNA-uEED constructs described above and compared to matching uEED design control GalNAc-GFP siRNA-uEED constructs, control GalNAc-siRNA Luc (no uEED), and untreated hepatocytes. Treated hepatocytes are monitored for RNAi knockdown of Luciferase by plate reader and IVIS imaging assays. All experiments are performed in triplicate and repeated on three independent days (biological triplicates of triplicates). It is expected that various GalNAc-Luc siRNA-uEED constructs will result in more efficient RNAi luciferase knockdown and thereby require a lower dose vs. control GalNAc-Luc siRNA (no uEED) control.

Delivery of RNA Oligonucleotides into Animal Models. To test the ability of uEEDs to enhance endosomal escape and delivery of RNA oligonucleotide therapeutics into tissues in preclinical animal models in vivo, Luciferase expressing preclinical mice are treated with various GalNAc-Luc siRNA-uEED constructs vs. controls.

ROSA26-Lox-Stop-lox (LSL) Luciferase mice are pretreated with an i.v. administered adenovirus-Cre to recombine out the LSL DNA segment and thereby constitutively express Luciferase in liver hepatocytes. Treated mice will be monitored daily by live animal IVIS imaging for constitutive baseline luciferase expression starting one-week post Adenovirus-Cre infection. To obtain a baseline measurement for all animals, animals are randomized into groups (n=8/group), injected with luciferin, and assayed by live animal IVIS bioluminescence imaging for three days prior to treatment (days −2, −1, 0). After imaging on day 0, mice are treated by either subcutaneous or i.v. administered GalNAc-Luc siRNA-uEED constructs containing from 2, 4, 6 . . . to 20 or more uEED monomers described above and compared to matching uEED design control GalNAc-GFP siRNA-uEED constructs, control GalNAc-siRNA Luc (no uEED), and untreated mice. All animal groups are assayed by live animal IVIS bioluminescence imaging following luciferin injection on days 1, 2, 3, 5, 7, 14, 21, 28 (and longer if necessary).

It is anticipated that various GalNAc-Luc siRNA-uEED constructs will result in more efficient RNAi luciferase knockdown and thereby requiring a lower dose vs. control GalNAc-Luc siRNA (no uEED) control and control GalNAc-GFP siRNA-uEED constructs.

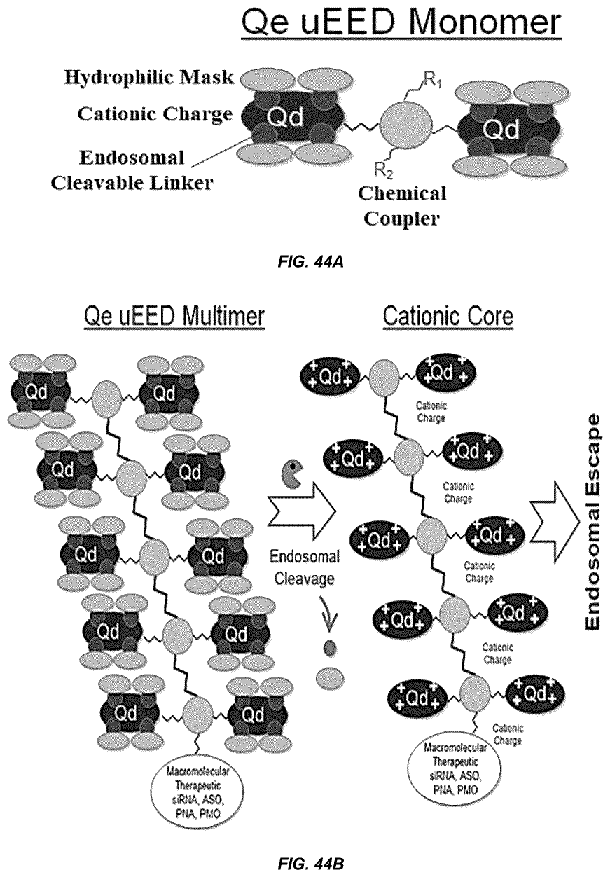

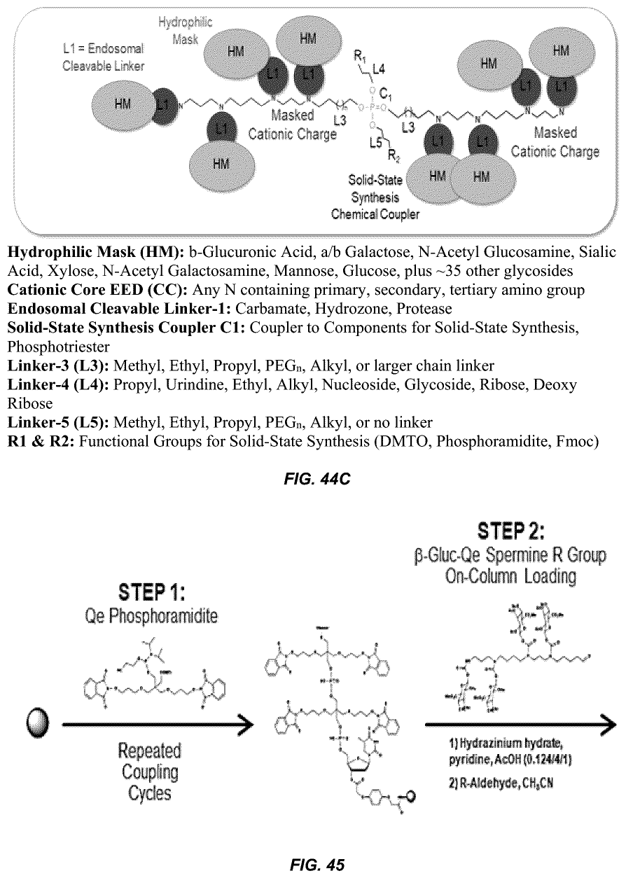

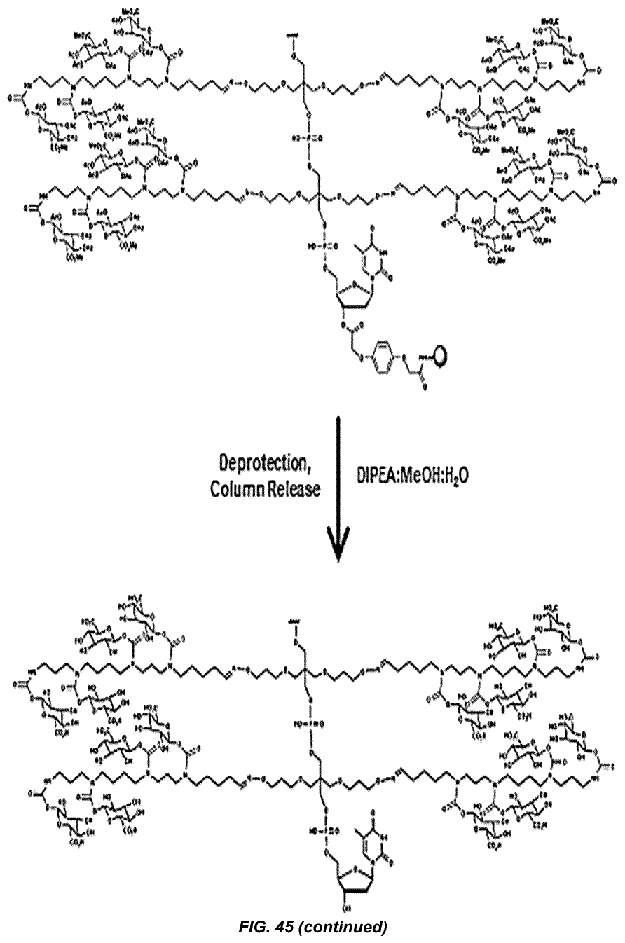

Qb6 uEED (6mer) was conjugated to the 5′ end of a Luciferase (Luc) passenger strand and then duplexed with a Guide strand to form siLuc-Qb6. 0.3 nmol of Qb6-siLuc5-cy3 was incubated in 50% human serum plus 50% saline incubated at 37° C. Final vol 20 ul. Samples mixed 1:1 with UREA gel loading buffer, then loaded on a 15% denaturing UREA-PAGE gel, followed by staining with methylene blue. Imaged on biorad chemidoc (FIG. 41). siLuc-Qb6 was placed into lysosomal conditions of 300 mM pH 5.0 sodium acetate buffer, β-Glucuronidase (10 U/ul), and 0.25 nmol Qb6-siLuc5 oligo incubated at 37°. Final vol. 15 ul. Samples mixed 1:1 with UREA gel loading buffer then loaded on a 15% denaturing UREA-PAGE gel, followed by staining with methylene blue. Imaged on biorad chemidoc (FIG. 42 and FIG. 43).

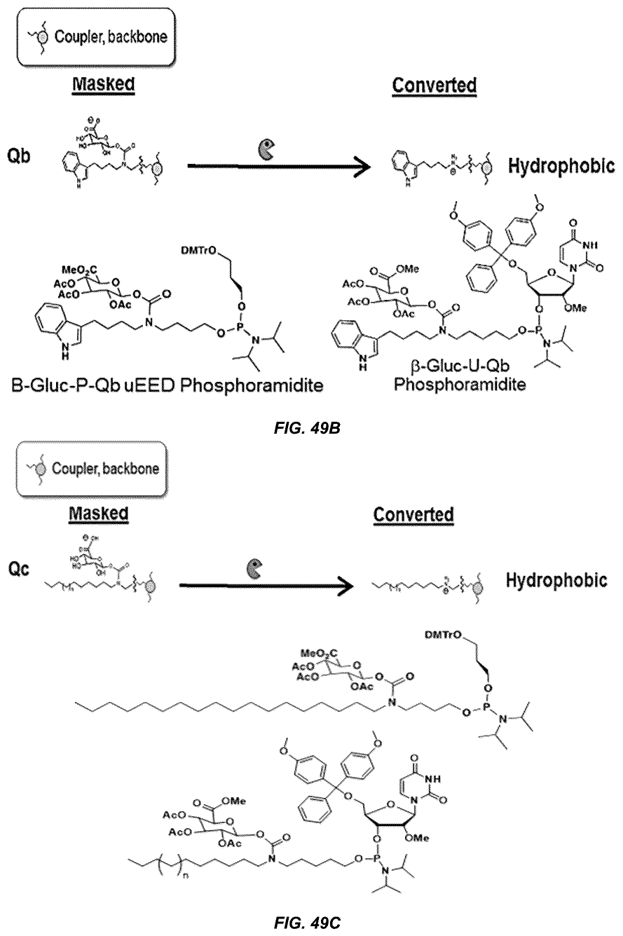

T15(Qd-b)2 oligo was synthesized as a single oligo (no conjugation) and 0.25 nmol of T15Qd2 oligo was tested for conversion under lysosomal conditions of 300 mM pH 5.0 sodium acetate buffer, β-Glucuronidase (10 U/ul), at 37° C. for 1 hour. Final vol 15 ul. Samples were mixed 1:1 with UREA gel loading buffer then loaded on a 15% denaturing UREA-PAGE gel, followed by staining with methylene blue. Imaged on biorad chemidoc (FIG. 46). The B-Glucuronidase treated and converted T15(Qd-b)2 tester oligo migrates more slowly because, even though it has lost some molecular mass by cleavage of 6 Glucuronic acids, it has gained a 6 positive charges that neutralize 6 of the negatively shared oligo backbone phosphodiesters, resulting in less charge to pull the oligo into the gel and hence a slower migration.

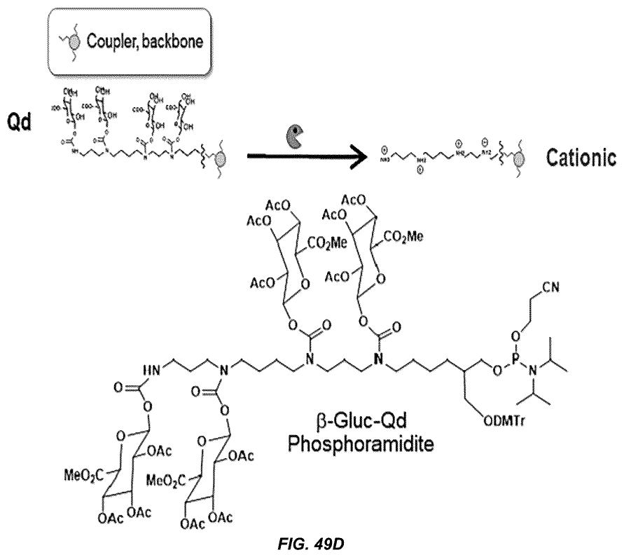

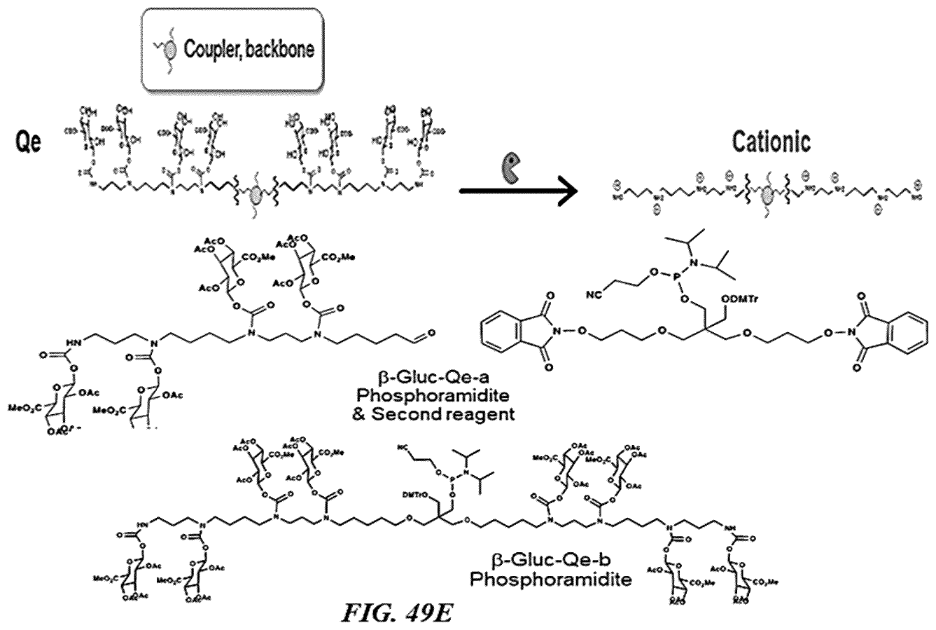

A trimer of GalNAc conjugated to an siRNA or ASO with uEED are generated. An <ED50 dose is used and a comparison of GalNAc-siRNN & ASO conjugates +/− uEED is performed. Wild type Balb/C mice are injected subcutaneously with GalNAc-siRNA and GalNAc-ASO conjugates. Blood is sampled prior to administration (day 0) and on days 3 and 6 (FIG. 47). Blood is analyzed by ELISA for the level of liver produced and secreted TTR and AT3 proteins is measured. Estimated and expected results are provided in FIG. 48, which will show a stronger knockdown in uEED conjugates due to rapid activation compared to controls.

The figures provide a number of variations of uEED constructs of the disclosure, the constructs are not to be limiting and are exemplary only. Moreover, each constructs is explicitly contemplated herein. In addition, a number of synthesis methods are provided in the figures; these methods are exemplary only and are not limiting.

A number of embodiments have been described. Nevertheless, it will be understood that various modifications may be made without departing from the spirit and scope of the description. Accordingly, other embodiments are within the scope of the following claims.

1. A monomeric compound comprising:
a coupler domain;
a hydrophobic domain or a cationic charge domain;
a hydrophilic domain;
a biodegradable linker having a first end and a second end, wherein the biodegradable linker is linked to the hydrophilic domain on the first end, and linked to the hydrophobic domain or cationic charge domain on the second end, or linked to an optional first linker on the second end;
the optional first linker having a first end and a second end, wherein the first linker is linked to the coupler domain on the first end, and linked on the second end to the hydrophobic domain or cationic charge domain, or linked to an optional second linker;
an optional second linker having a first end and a second end, wherein the second linker is linked to the hydrophobic domain or a cationic charge domain on the first end, and linked to the first linker on the second end;
optionally, a third and/or fourth linkers having a first end and a second end, wherein the first end is attached to the coupler domain, wherein the second end is attached to a functional group for solid-state synthesis
optionally, a fifth linker having a first end and a second end, wherein the first end is attached to the hydrophobic domain or cationic charge domain, wherein the second end is attached to additional hydrophobic domain or cationic charge domain.

2. The monomeric compound of claim 1, wherein the compound has the structure of Formula I, II, III, IV, V or VI: 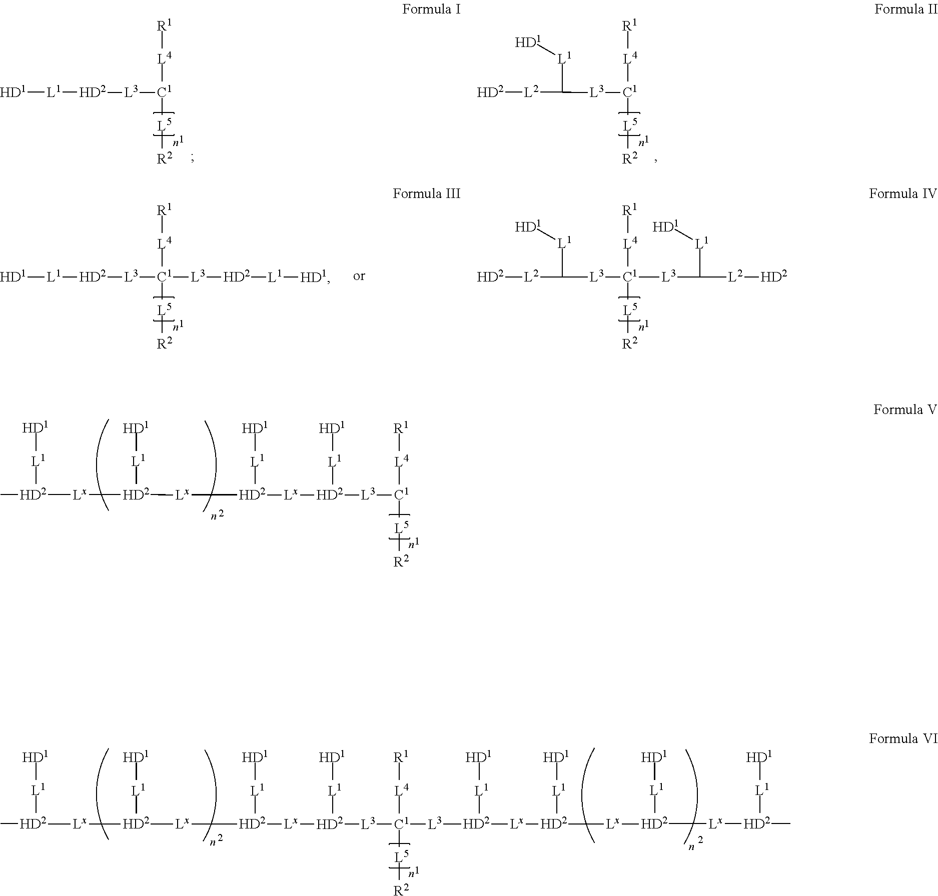
or a pharmaceutically acceptable salt, or solvate thereof,
wherein,
C¹ is the coupler domain;
HD¹ is the hydrophilic domain;
HD² is the hydrophobic domain or a cationic charge domain;
L¹ is the biodegradable linker;
L² is the optional second linker;
L³ is the optional first linker;
L⁴ is the optional third linker;
L⁵ is the optional fourth linker;
Lx is the optional fifth linker;
R¹ and R² are protecting or functional groups for solid-state synthesis;
n¹ is an integer selected from 0 or 1; and
n² is an integer selected from 0 to 10.

3. The monomeric compound of claim 1, wherein the coupler domain comprises a phosphotriester group, or phosphoramidite group.

4. The monomeric compound of claim 1, wherein the hydrophilic domain comprises a glycoside moiety.

5. The monomeric compound of claim 1, wherein the hydrophobic domain or the cationic charge domain is any functional group which contains an aromatic indole ring; nitrogen containing mono-cyclic or multi-cyclic rings; primary, secondary or tertiary amino group; a lipid or a monomeric unit derived therefrom; a tocopherol; a hydrophobic oligomer or a monomeric unit derived therefrom; a hydrophobic polymer or a monomeric unit derived therefrom.

6. The monomeric compound of claim 5, wherein the hydrophobic domain comprises a lipid selected from a C8, C10, C12, C14, C16, or C18 lipid or derivative thereof.

7. (canceled)

8. The monomeric compound of claim 5, wherein the hydrophobic domain comprises a hydrophobic polymer.

9. The monomeric compound of claim 5, wherein the hydrophobic domain comprises one or more monomeric units derived from a hydrophobic polymer selected from the group consisting of: polyester, polyether, polycarbonate, polyanhydride, polyamide, polyacrylate, polymethacrylate, polyacrylamide, polysulfone, polyalkane, polyalkene, polyalkyne, polyanhydride, polyorthoester, N-isopropylacrylamide, methyl acrylate, ethyl acrylate, propyl acrylate, butyl acrylate, methyl methacrylate, ethyl methacrylate, propyl methacrylate, butyl methacrylate, acrylic acid, methacrylic acid, quaternary ammonium-modified acrylate, quaternary ammonium modified-methacrylate, acrylamide, caprolactone, lactide, and valerolactone.

10. The monomeric compound of claim 5, wherein the hydrophobic domain or the cationic charge domain comprises a 1H-indole group; a nitrogen containing mono- or multicyclic aromatic or non-aromatic compounds.

11. The monomeric compound of claim 1, wherein the cationic charge domain comprises a primary amine, a secondary amine, a tertiary amine, a quaternary amine, a complex amino group, or an ionizable amines.

12. The monomeric compound of claim 11, wherein the cationic charge domain comprises metformin group, a morpholine group, a piperazine group, a pyridine group, a pyrrolidine group, piperidine, a thiomorpholine, a thiomorpholine oxide, a thiomorpholine dioxide, imidazole, guanidine or creatine.

13. (canceled)

14. The monomeric compound of claim 1, wherein the biodegradable linker comprises a thioether group, a carbamate group, a hydrazone group, an ester group, a carbonate group, a urea group, or an enzyme cleavable peptidic linkage.

15-16. (canceled)

17. The monomeric compound of claim 1, wherein the first linker, second linker, third linker and fourth linkers each independently comprises a group selected from an optionally substituted (C₁-C₆)alkyl, an optionally substituted (C₂-C₆)alkenyl, an optionally substituted (C₂-C₆)alkynyl, or an optionally substituted (C₁-C₆) alkoxy group.

18. The monomeric compound of claim 17, wherein the first linker and/or second linker comprises a group selected from ethyl, propyl, PEG₂, PEG₃ and PEG₄.

19-21. (canceled)

22. The monomeric compound of claim 17, wherein the third and fourth linkers are a (C₁-C₆) alkyl or a uridine group.

23. The monomeric compound of claim 1, wherein the functional or protecting groups for solid-state synthesis are an amidite and/or a 4,4′-dimethoxytrityl group.

24. The monomeric compound of claim 1, wherein the compound has a structure selected from:

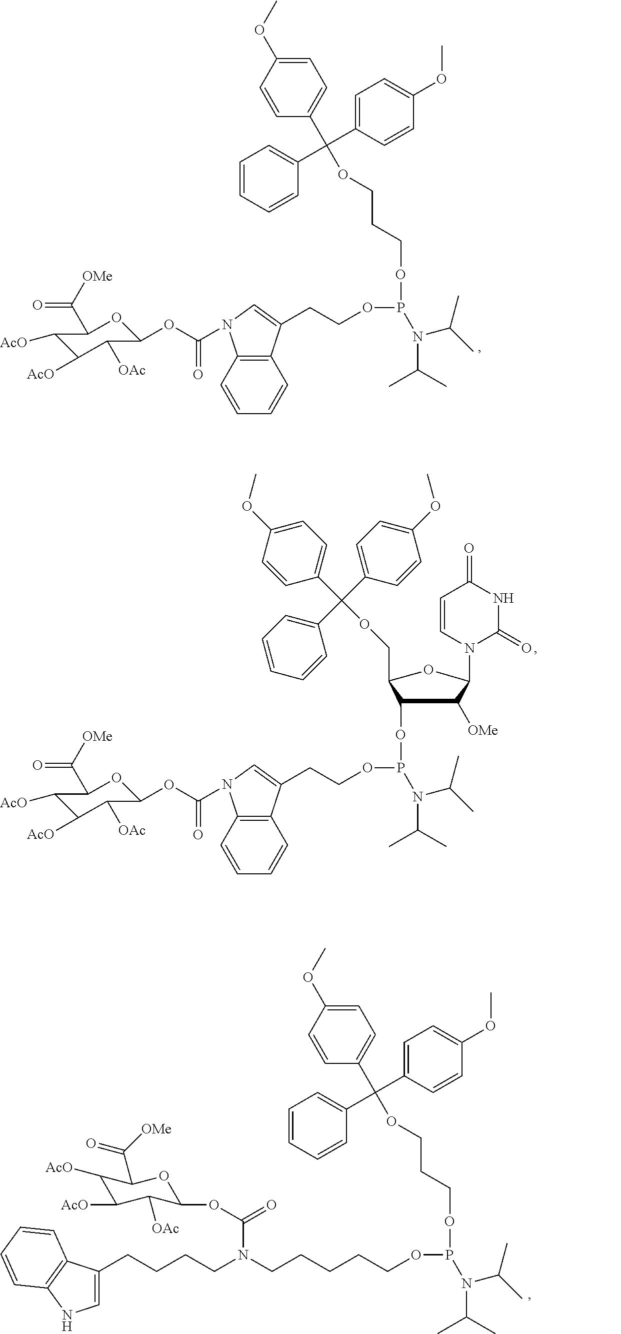

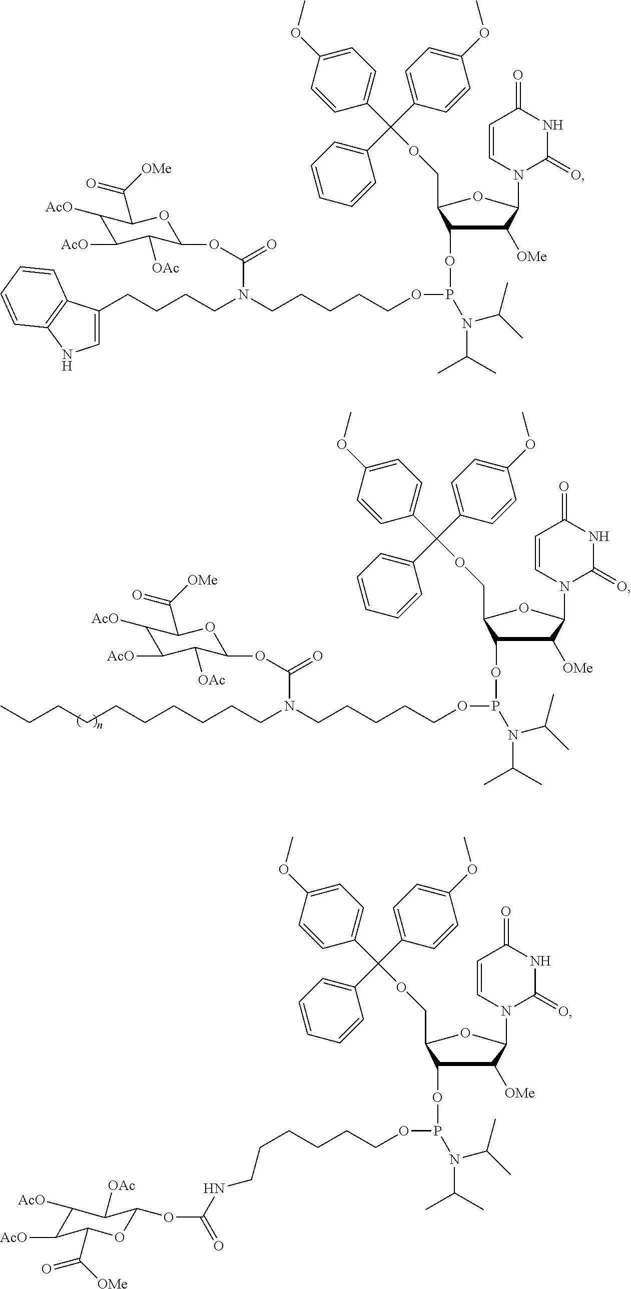

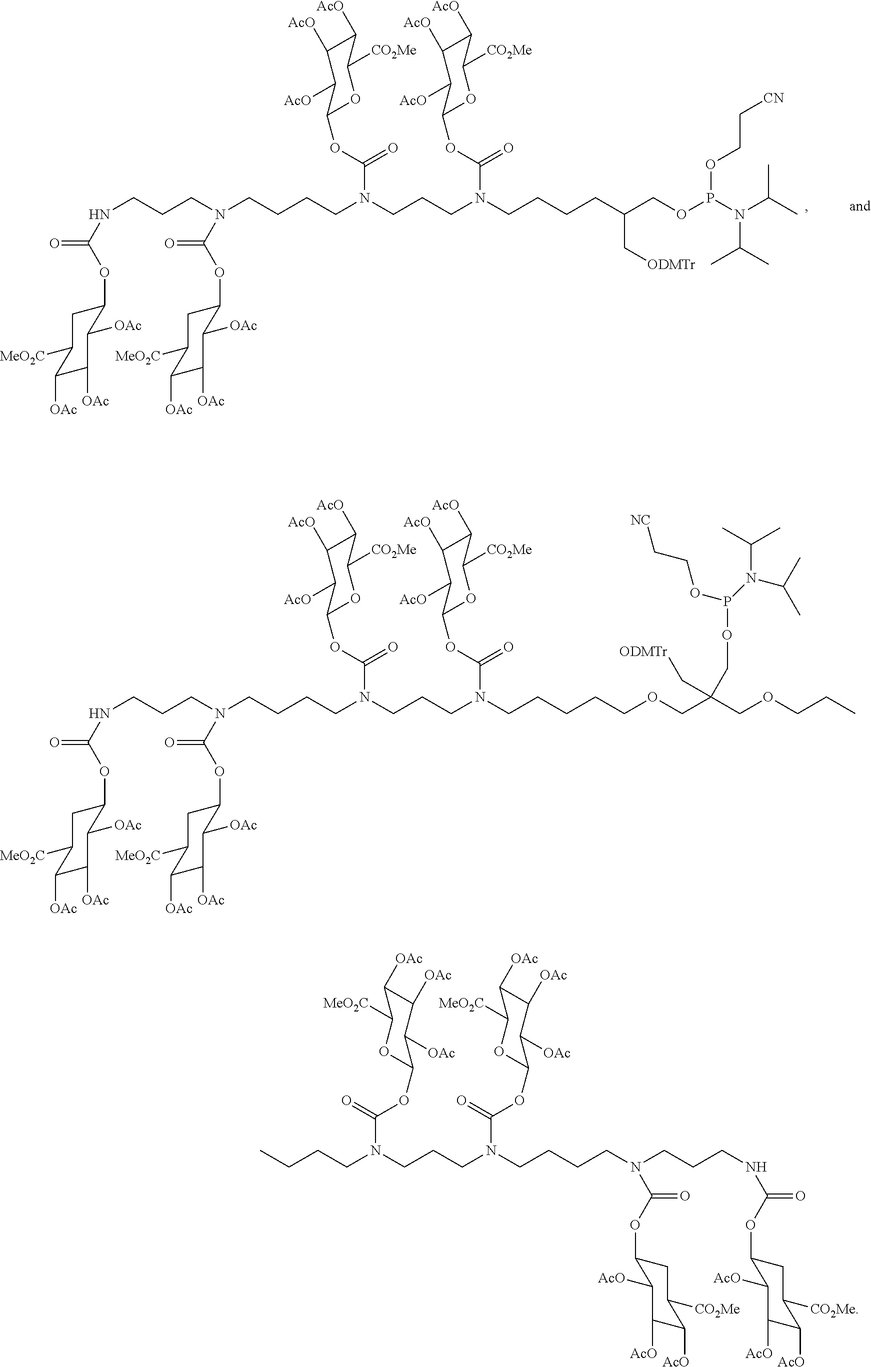

25. A multimeric compound comprising a plurality of monomeric compounds of claim 1, wherein the plurality of monomeric compounds have been linked together using solid-state synthesis to form a multimeric compound.

26. The multimeric compound of claim 25, wherein the multimeric compound is linked to a cargo molecule.

27. The multimeric compound of claim 26, wherein the cargo molecule is selected from the group consisting of a small molecule therapeutic, a peptide, a protein, a single stranded oligonucleotide, a double stranded oligonucleotide, and a protein-oligonucleotide complex.

28. (canceled)

29. The multimeric compound of claim 25 having a structure of Formula VII: 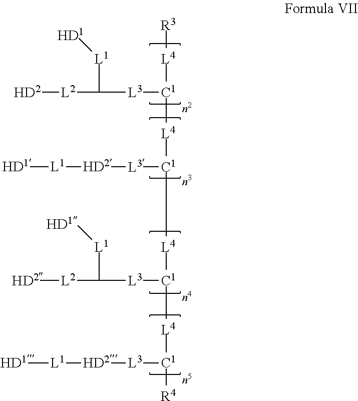
or a pharmaceutically acceptable salt, or solvate thereof,
wherein,
C¹ is a coupler domain;
HD¹, HD^{1′}, HD^{1″}, and HD^{1′″} are each individually selected hydrophilic domains;
HD², HD^{2′}, HD^{2″}, and HD^{2′″} are each individually selected hydrophobic domains or cationic charge domains;
L^{1′} is a biodegradable linker;
L² is a second linker;
L³ is a first linker;
L⁴ is a third linker;
R³ is an H or a conjugation handle for a cargo molecule;
R⁴ is an H or a conjugation handle for a cargo molecule;
n² is an integer selected from 0 to 10;
n³ is an integer selected from 0 to 10;
n⁴ is an integer selected from 0 to 10; and
n⁵ is an integer selected from 0 to 10;
wherein, the summation of the integers specified for n¹ to n⁵ is from 4 to 30, and
wherein at least one of R³ and R⁴ is a conjugation handle for a cargo molecule.

30. The multimeric compound of claim 29, wherein the coupler domain comprises a phosphotriester group.

31. The multimeric compound of claim 29, wherein the hydrophilic mask domains comprise a glycoside moiety.

32. The multimeric compound of claim 29, wherein the hydrophobic domains or the cationic charge domains are selected from any functional group which contains a primary, secondary or tertiary amine group; a lipid or a monomeric unit derived therefrom; a tocopherol; a hydrophobic oligomer or a monomeric unit derived therefrom; a hydrophobic polymer or a monomeric unit derived therefrom.

33. The multimeric compound of claim 32, wherein one or more of the hydrophobic domains comprise a lipid selected from a C8, C10, C12, C14, C16, or C18 lipid or derivative thereof.

34. (canceled)

35. The multimeric compound of claim 32, wherein one or more of the hydrophobic domains comprise a hydrophobic polymer selected from polymethylacryl, polyethylene, polystyrene, polyisobutane, polyester, polypeptide, or a derivative thereof.

36. (canceled)

37. The multimeric compound of claim 32, wherein one or more of the hydrophobic domains or the cationic charge domains comprise a 1H-indole group.

38. The multimeric compound of claim 32, wherein the cationic charge domain comprises a primary amine, a secondary amine, a tertiary amine, a quaternary amine, a complex amino group, or an ionizable amine.

39. The multimeric compound of claim 38, wherein the cationic charge domain comprises metformin group, a morpholine group, a piperazine group, a pyridine group, a pyrrolidine group, piperidine, a thiomorpholine, a thiomorpholine oxide, a thiomorpholine dioxide, imidazole, guanidine or creatine.

40. (canceled)

41. The multimeric compound of claim 29, wherein the biodegradable linker comprises a thioether group, a carbamate group, a hydrazone group, an ester group, a carbonate group, a urea group, or an enzyme cleavable peptidic linkage.

42-43. (canceled)

44. The multimeric compound of claim 29, wherein the first linker, second linker, and third linker each independently comprises a group selected from an optionally substituted (C₁-C₆)alkyl, an optionally substituted (C₂-C₆)alkenyl, an optionally substituted (C₂-C₆)alkynyl, or an optionally substituted (C₁-C₆) alkoxy group.

45. The multimeric compound of claim 44, wherein the first and second linker comprises a group selected from ethyl, propyl, PEG₂, PEG₃ and PEG₄.

46-48. (canceled)

49. The multimeric compound of claim 44, wherein the third linker is a (C₁-C₆) alkyl or a uridine group.

50. The multimeric compound of claim 29, wherein the conjugation handle for a cargo molecule comprises an azide group.

51. The multimeric compound of claim 29, wherein the conjugation handle for a cargo molecule comprises a structure of: 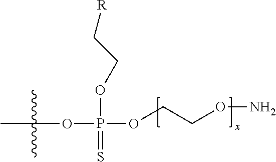
wherein,
x is an integer selected from 1 to 15; and
R is —OH, or —CN.

52. The multimeric compound of claim 29, wherein the multimeric compound is linked to a cargo molecule.

53. The multimeric compound of claim 52, wherein the cargo molecule is selected from the group consisting of a small molecule therapeutic, a peptide, a protein, a single stranded oligonucleotide, a double stranded oligonucleotide, and a protein-oligonucleotide complex.

54. (canceled)

55. The monomeric compound of claim 1, further comprising a targeting moiety linked to the compound.

56. The monomeric compound of claim 55, wherein the targeting domain is linked to the hydrophilic domain.

57. The monomeric compound of claim 56, wherein the targeting compound causes endocytosis or is endocytosed by a cell.

58. The monomeric compound of claim 55, wherein the targeting moiety specifically binds to a protein selected from the group comprising insulin, insulin-like growth factor receptor 1 (IGF1R), IGF2R, insulin-like growth factor (IGF), mesenchymal epithelial transition factor receptor (c-met; also known as hepatocyte growth factor receptor (HGFR)), hepatocyte growth factor (HGF), epidermal growth factor receptor (EGFR), epidermal growth factor (EGF), heregulin, fibroblast growth factor receptor (FGFR), platelet-derived growth factor receptor (PDGFR), platelet-derived growth factor (PDGF), vascular endothelial growth factor receptor (VEGFR), vascular endothelial growth factor (VEGF), tumor necrosis factor receptor (TNFR), tumor necrosis factor alpha (TNF-α), TNF-β, folate receptor (FOLR), folate, transferring, transferrin receptor (TfR), mesothelin, Fc receptor, c-kit receptor, c-kit, an integrin (e.g., an α4 integrin or a β-1 integrin), P-selectin, sphingosine-1-phosphate receptor-1 (S1PR), hyaluronate receptor, leukocyte function antigen-1 (LFA-1), CD4, CD11, CD18, CD20, CD25, CD27, CD52, CD70, CD80, CD85, CD95 (Fas receptor), CD106 (vascular cell adhesion molecule 1 (VCAM1), CD166 (activated leukocyte cell adhesion molecule (ALCAM)), CD178 (Fas ligand), CD253 (TNF-related apoptosis-inducing ligand (TRAIL)), ICOS ligand, CCR2, CXCR3, CCR5, CXCL12 (stromal cell-derived factor 1 (SDF-1)), interleukin 1 (IL-1), IL-1ra, IL-2, IL-3, IL-4, IL-6, IL-7, IL-8, CTLA-4, MART-1, gp100, MAGE-1, ephrin (Eph) receptor, mucosal addressin cell adhesion molecule 1 (MAdCAM-1), carcinoembryonic antigen (CEA), Lewis^{Y}, MUC-1, epithelial cell adhesion molecule (EpCAM), cancer antigen 125 (CA125), prostate specific membrane antigen (PSMA), TAG-72 antigen, and fragments thereof.

59. (canceled)

60. The monomeric compound of claim 55, wherein the targeting moiety is an antibody or antibody fragment, or a ligand that binds to a cell surface receptor.

61. A method of delivering a cargo moiety to a cell, the method comprising contacting the cell with a construct comprising a targeting moiety that causes endocytosis or is endocytosed, wherein the targeting moiety is linked to a hydrophilic domain of a monomeric compound of claim 1 and wherein the monomeric unit is linked to the cargo domain.

62. The method of claim 61, wherein the cargo moiety is a small molecule therapeutic, a peptide, a protein, a single stranded oligonucleotide, a double stranded oligonucleotide, and a protein-oligonucleotide complex.

63-70. (canceled)

71. A compound comprising:
a targeting domain;
a cargo domain;
a coupler domain;
a hydrophobic domain or a cationic charge domain;
a hydrophilic domain;
a biodegradable linker having a first end and a second end, wherein the biodegradable linker is linked to the hydrophilic domain on the first end, and linked to the hydrophobic domain or cationic charge domain on the second end, or linked to an optional first linker on the second end;
the optional first linker having a first end and a second end, wherein the first linker is linked to the coupler domain on the first end, and linked on the second end to the hydrophobic domain or cationic charge domain, or linked to an optional second linker;
an optional second linker having a first end and a second end, wherein the second linker is linked to the hydrophobic domain or a cationic charge domain on the first end, and linked to the first linker on the second end;
optionally, a third and/or fourth linkers having a first end and a second end, wherein the first end is attached to the coupler domain, wherein the second end is attached to a functional group for solid-state synthesis
optionally, a fifth linker having a first end and a second end, wherein the first end is attached to the hydrophobic domain or cationic charge domain, wherein the second end is attached to additional hydrophobic domain or cationic charge domain
wherein the targeting domain is linked to the hydrophilic domain or the cargo domain; and
wherein the cargo domain is linked to the coupler domain.
